(19) United States
(12) Patent Application Publication
(10) Pub. No.: US 2022/0112497 A1
(43) Pub. Date: Apr. 14, 2022

(54) CANCER-SPECIFIC MOLECULES AND METHODS OF USE THEREOF

(71) Applicant: Envisagenics, Inc., New York, NY (US)

(72) Inventors: Paulina Zheng, New York, NY (US); Anson Abraham, Roslyn Heights, NY (US); Maria Luisa Pineda, Long Island City, NY (US); Martin Akerman, Huntington, NY (US); Gayatri Arun, Huntington Station, NY (US); Priyanka Dhingra, Jersey City, NJ (US); Kendall Anderson, New York, NY (US); Vanessa Frederick, Brooklyn, NY (US); Naomi Yudanin, New York, NY (US); Robin Munch, New York, NY (US)

(21) Appl. No.: 17/495,266

(22) Filed: Oct. 6, 2021

Related U.S. Application Data
(63) Continuation of application No. PCT/US2020/027534, filed on Apr. 9, 2020.
(60) Provisional application No. 62/831,604, filed on Apr. 9, 2019.
(60) Provisional application No. 62/889,217, filed on Aug. 20, 2019.
(60) Provisional application No. 62/944,913, filed on Dec. 6, 2019.
(60) Provisional application No. 62/980,900, filed on Feb. 24, 2020.

(51) Int. Cl. C12N 15/113

(52) U.S. Cl. CPC C12N 15/113; C12N 2310/11; C12N 2320/33; C12N 2310/321; C12N 2310/315

(57) ABSTRACT

Disclosed herein are systems and methods for identifying candidate targets that may be used for therapeutic developments. The systems and methods may comprise receiving and analyzing biologically relevant data to identify information or events such as splicing events that may be statistically significant or specific to certain diseases, illnesses or conditions. Also provided are systems and methods for modulating the statistically significant events using compositions or molecules including small molecule compounds, oligonucleotides, proteins or cells.

### CROSS-REFERENCE

This application claims the benefit of U.S. Provisional Patent Application No. 62/831,604, filed Apr. 9, 2019, U.S. Provisional Patent Application No. 62/889,217, filed Aug. 20, 2019, U.S. Provisional Patent Application No. 62/944,913, filed Dec. 6, 2019, and U.S. Provisional Patent Application No. 62/980,900, filed Feb. 24, 2020, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on 16 Dec. 2021, is named EVG-002WOC1_SL.txt and is 1,282,000 bytes in size.

### BACKGROUND

Cancer and genetic diseases affect more than millions of people in the U.S. Splicing deregulation can be a major hallmark of cancer and genetic diseases, affecting progression, metastasis, and therapy resistance. RNA splicing is the process by which introns, the non-protein coding regions of DNA, are removed from nascent precursor messenger RNA (pre-mRNA), and exons, the protein coding regions of DNA, are joined together to form mature messenger RNA (mRNA). RNA splicing errors may result in spliced RNA that fail to produce functional proteins, thereby causing genetic diseases including many types of cancers.

### SUMMARY

RNA splicing is a form of RNA processing in which a newly made precursor messenger RNA (pre-mRNA) transcript is transformed into a mature messenger RNA (mRNA). During splicing, introns (intra-genic, non-coding regions) are removed and exons (coding regions) are joined together. RNA splicing not only provides functional mRNA, but may also be responsible for generating additional diversity (i.e., alternative splicing, alternative RNA splicing, or differential splicing). The alternative splicing may result in the production of different mRNAs from the same gene. The mRNAs that represent isoforms arising from a single gene can differ by the use of alternative exons or retention of an intron that disrupts two exons. This process often leads to different protein products that may have related or drastically different, even antagonistic, cellular functions. The alternative splicing may comprise one or more of exon skipping or cassette exon, mutually exclusive exons, alternative donor site, alternative acceptor site, intron retention, and any combination or variation thereof.

Multiple studies have shown the oncogenic activity of specific splicing events and splicing factors, such as SRSF1, SRSF2, ESRP1, and RBFOX1, in human and animal models. As such, cancer-associated splicing deregulation may be a novel source of clinically-applicable biomarkers and therapeutic targets.

Provided herein are systems and methods for identifying therapeutic targets (e.g., disease-specific splicing events) in various diseases or illnesses such as cancers. Non-limiting examples of cancers may include prostate cancer, cancers of barrier tissues (i.e. colorectal cancer, lung cancer) and in other TGFb-rich sites like ovaries, AML/hematological disorders, respiratory system cancer, hepatocellular carcinoma (liver cancer) which may include both a TGFb-rich environment and chronic, potentially diet induced inflammation, thoracic cancer, stomach cancer, kidney cancer, pancreatic cancer, skin cancer, or combinations thereof. Examples of some other diseases may include, but are not limited to, autism (i.e., ST7 (ENV19)), lymphatic disease such as syndromic lymphedema-genetic disorder and milory disease, eye degenerative diseases, brain disease- peripheral neuropathy, neurometabolic disease, rare genetic neurological disorders-genetic motor neuron disease, psychiatric disorders, chronic inflammation/autoimmune disease, including IBD, crohn's disease, and similar gastrointestinal disorders, inflammation in pathogenic obesity, including hereditary, childhood, leptin and non-leptin dependent disease, hypertension and/or other comorbidities associated with western diets.

The methods of the present disclosure may also comprise detecting one or more biomarkers, for example, detecting a presence or an absence of specific DNA sequence, mRNA and/or protein isoforms. The presence or absence of the one or more biomarkers can be used to diagnose disease and/or track disease progression.

Also provided herein are methods for modulating therapeutic targets (e.g., disease-specific splicing events) using various types of modalities, such as oligonucleotides, small molecules, antibodies, or any combination thereof. In some examples, the modalities may switch pathogenic RNA isoforms to non-pathogenic RNA isoforms. In some examples, the modalities are oligonucleotides including splicing-switch oligonucleotides (SSO), antisense oligonucleotides (ASO), small interfering Ribonucleic Acid (siRNA), conjugated oligonucleotides, or a combination thereof that can knockdown specific isoforms. In some other example, the modalities are antibodies or cell-based (e.g., CAR-T) that may specifically recognize protein isoform of alternatively spliced RNA, and/or therapeutic compounds including ASO, small molecules or biologics that target the isoforms specifically to obtain therapeutic benefit.

Further provided in the present disclosure are methods and systems for treating diseases or illnesses such as cancers. The systems and methods may comprises administering an effective amount to modalities to a subject having the diseases or illnesses. The modalities may be oligonucleotides, small molecules, antibodies, or any combination thereof, which are designed to achieve disease-specific targeting. For example, some of the disease-specific splicing events that have been identified can open up grooves for small molecule binding. In that case, small molecules can be designed to target the region that is created because of a splicing change. In another example, splicing changes of the membrane bound proteins can display altered surface epitopes that can be specifically targeted using antibodies.

An aspect of the present disclosure provides a method of modulating splicing in a pre-mRNA in a biological sample comprising: contacting the biological sample with an antisense compound comprising one or more oligonucleotides having at least 90% sequence identity to oligonucleotides selected from Tables 2-33.

In some embodiments, the biological sample comprises a cell, a tissue, or a blood sample. In some embodiments, the biological sample is in vitro. In some embodiments, the biological sample is a cell. In some embodiments, the method further comprises measuring viability of the cell. In some embodiments, the measuring is over a predetermined time period. In some embodiments, the method further comprises monitoring the viability of the cell over a predetermined time period. In some embodiments, the method further comprises decreasing or increasing a concentration of the antisense compound based on the viability of the cell. In some embodiments, the method further comprises decreasing or increasing a concentration of the antisense compound when the viability of the cell is below a cut-off value. In some embodiments, the cut-off value is about 80%. In some embodiments, the cut-off value is about 90%. In some embodiments, the antisense compound comprises the one or more oligonucleotides at a concentration of greater than or equal to about 300 nM. In some embodiments, the antisense compound comprises the one or more oligonucleotides at a concentration of less than or equal to about 450 nM. In some embodiments, the one or more oligonucleotides comprise deoxyribonucleic acid (DNA), ribonucleic acid (RNA), or a combination thereof. In some embodiments, the RNA comprises small interfering RNA (siRNA). In some embodiments, the one or more oligonucleotides comprises single-stranded oligonucleotides, double-stranded oligonucleotides, or a combination thereof. In some embodiments, the biological sample is from a subject having or suspected of having a disease or condition. In some embodiments, the disease or condition comprises a genetic disease, a CNS disease, an inflammatory disease, a neurodegenerative disease, a cardiovascular disease, an autoimmune disease, or cancer. In some embodiments, the disease is cancer. In some embodiments, the cancer comprises lung cancer, kidney cancer, or breast cancer. In some embodiments, the breast cancer is triple-negative breast cancer. In some embodiments, the one or more nucleotides comprise oligonucleotides selected from Tables 2-33. In some embodiments, the one or more oligonucleotides comprise a modified oligonucleotide. In some embodiments, the modified oligonucleotide comprises at least one modified internucleoside linkage In some embodiments, the at least one modified internucleoside linkage is phosphorothioate linkage In some embodiments, the modified oligonucleotide comprises one or more modified nucleotides. In some embodiments, the modified oligonucleotide comprises one or more modified nucleosides. In some embodiments, a modified nucleoside of the one or more modified nucleosides comprises a modified sugar moiety. In some embodiments, the modified sugar moiety is a 2′-substituted sugar moiety. In some embodiments, the 2′-substituted sugar moiety comprises a modification selected from the group consisting of 2′-O-methoxyethyl, 2′-fluoro, 2′-dimethylaminooxyethoxy, 2′-dimethylaminoethoxyethoxy, 2′-guanidinium, 2′-O-guanidinium ethyl, 2′-carbamate, 2′aminooxy, 2′-acetamido, and locked nucleic acid. In some embodiments, the modified oligonucleotide comprises a plurality of modified nucleosides each comprising a modified sugar moiety. In some embodiments, at least a subset of the plurality of modified nucleosides are different from one another. In some embodiments, the modulating comprises inducing or enhancing exon skipping. In some embodiments, the modulating comprises inducing or enhancing exon inclusion. In some embodiments, the modulating comprises promoting a splicing switch. In some embodiments, the modulating comprises down-regulation or up-regulation of splicing. In some embodiments, the antisense compound specifically binds to a segment of a pre-mRNA encoded by a gene comprising NEDD4L, MAP3K7, NFYA, ESYT2, MARK2, ST7, ARVCF, SYTL2, R3HDM1,COL4A3BP, TANGO2, SEPT9, ROBO1,FAM122B, CD47, LSR, PBX1,EPB41, ADAM15, EPB41L1, ABI1, FLNB, CTNND1, GPR160, ITGB3BP, INCENP, DENND1B, or CA12.

Another aspect of the present disclosure provides a pharmaceutical composition comprising (i) an antisense compound comprising one or more oligonucleotides having at least 90% sequence identity to oligonucleotides selected from Tables 2-33, and (ii) a pharmaceutically acceptable diluent or carrier.

In some embodiments, the antisense compound comprises the one or more oligonucleotides at a concentration of greater than or equal to about 300 nM. In some embodiments, the antisense compound comprises the one or more oligonucleotides at a concentration of less than or equal to about 450 nM. In some embodiments, the one or more oligonucleotides comprise deoxyribonucleic acid (DNA), ribonucleic acid (RNA), or a combination thereof. In some embodiments, the RNA comprises small interfering RNA (siRNA). In some embodiments, the one or more oligonucleotides comprises single-stranded oligonucleotides, double-stranded oligonucleotides, or a combination thereof. In some embodiments, the pharmaceutical composition is used for treating or alleviating a disease or condition. In some embodiments, the disease comprises a genetic disease, a CNS disease, an inflammatory disease, a neurodegenerative disease, a cardiovascular disease, an autoimmune disease, or cancer. In some embodiments, the disease or condition is cancer. In some embodiments, the cancer comprises lung cancer, kidney cancer, or breast cancer. In some embodiments, the breast cancer is triple-negative breast cancer. In some embodiments, the one or more nucleotides comprise oligonucleotides selected from Tables 2-33. In some embodiments, the one or more oligonucleotides comprise a modified oligonucleotide. In some embodiments, the modified oligonucleotide comprises at least one modified internucleoside linkage In some embodiments, the at least one modified internucleoside linkage is phosphorothioate linkage In some embodiments, the modified oligonucleotide comprises one or more modified nucleotides. In some embodiments, the modified oligonucleotide comprises one or more modified nucleosides. In some embodiments, a modified nucleoside of the one or more modified nucleosides comprises a modified sugar moiety. In some embodiments, the modified sugar moiety is a 2′-substituted sugar moiety. In some embodiments, the 2′-substituted sugar moiety comprises a modification selected from the group consisting of 2′-O-methoxyethyl, 2′-fluoro, 2′-dimethylaminooxyethoxy, 2′-dimethylaminoethoxyethoxy, 2′-guanidinium, 2′-O-guanidinium ethyl, 2′-carbamate, 2′aminooxy, 2′-acetamido, and locked nucleic acid. In some embodiments, the modified oligonucleotide comprises a plurality of modified nucleosides each comprising a modified sugar moiety. In some embodiments, at least a subset of the plurality of modified nucleosides are different from one another. In some embodiments, the pharmaceutical composition is used for modulating splicing of a pre-mRNA encoded by a gene comprising NEDD4L, MAP3K7, NFYA, ESYT2, MARK2, ST7, ARVCF, SYTL2, R3HDM1, COL4A3BP, TANGO2, SEPT9, ROBO1, FAM122B, CD47, LSR, PBX1, EPB41, ADAM15, EPB41L1, ABI1, FLNB, CTNND1, GPR160, ITGB3BP, INCENP, DENND1B, or CA12. In some embodiments, the modulating comprises inducing or enhancing exon skipping. In some embodiments, the modulating comprises inducing or enhancing exon inclusion. In some embodiments, the modulating comprises promoting a splicing switch. In some embodiments, the modulating comprises down-regulation or up-regulation of splicing.

Another aspect of the present disclosure provides an antisense compound for use in preparation of a medicament for the treatment of a disease or a condition, the antisense compound comprising one or more oligonucleotides having at least 90% sequence identity to oligonucleotides selected from Tables 2-33.

In some embodiments, the antisense compounds comprise the one or more oligonucleotides at a concentration of greater than or equal to about 300 nM. In some embodiments, the antisense compound comprises the one or more oligonucleotides at a concentration of the concentration is less than or equal to about 450 nM. In some embodiments, the one or more oligonucleotides comprise deoxyribonucleic acid (DNA), ribonucleic acid (RNA), or a combination thereof. In some embodiments, the RNA comprises small interfering RNA (siRNA). In some embodiments, the one or more oligonucleotides comprises single-stranded oligonucleotides, double-stranded oligonucleotides, or a combination thereof. In some embodiments, the disease or condition comprises a genetic disease, a CNS disease, an inflammatory disease, a neurodegenerative disease, a cardiovascular disease, an autoimmune disease, or cancer. In some embodiments, the disease is cancer. In some embodiments, the cancer comprises lung cancer, kidney cancer, or breast cancer. In some embodiments, the breast cancer is triple-negative breast cancer. In some embodiments, the one or more nucleotides comprise oligonucleotides selected from Tables 2-33. In some embodiments, the one or more oligonucleotides comprise a modified oligonucleotide. In some embodiments, the modified oligonucleotide comprises at least one modified internucleoside linkage. In some embodiments, the at least one modified internucleoside linkage is phosphorothioate linkage. In some embodiments, the modified oligonucleotide comprises one or more modified nucleotides. In some embodiments, the modified oligonucleotide comprises one or more modified nucleosides. In some embodiments, a modified nucleoside of the one or more modified nucleosides comprises a modified sugar moiety. In some embodiments, the modified sugar moiety is a 2′-substituted sugar moiety. In some embodiments, the 2′-substituted sugar moiety comprises a modification selected from the group consisting of 2′-O-methoxyethyl, 2′-fluoro, 2′-dimethylaminooxyethoxy, 2′-dimethylaminoethoxyethoxy, 2′-guanidinium, 2′-O-guanidinium ethyl, 2′-carbamate, 2′aminooxy, 2′-acetamido and locked nucleic acid. In some embodiments, the modified oligonucleotide comprises a plurality of modified nucleosides each comprising a modified sugar moiety. In some embodiments, at least a subset of the plurality of modified nucleosides are different from one another. In some embodiments, the treatment comprising modulating splicing of a pre-mRNA encoded by a gene comprising NEDD4L, MAP3K7, NFYA, ESYT2, MARK2, ST7, ARVCF, SYTL2, R3HDM1, COL4A3BP, TANGO2, SEPT9, ROBO1, FAM122B, CD47, LSR, PBX1, EPB41, ADAM15,EPB41L1, ABI1, FLNB,CTNND1, GPR160, ITGB3BP, INCENP,DENND1B, or CA12. In some embodiments, the modulating comprises inducing or enhancing exon skipping. In some embodiments, the modulating comprises inducing or enhancing exon inclusion. In some embodiments, the modulating comprises promoting a splicing switch. In some embodiments, the modulating comprises down-regulation or up-regulation of splicing.

Another aspect of the present disclosure provides a method of modulating splicing in a pre-mRNA in a biological sample comprising: contacting the biological sample with a composition which specifically binds to a segment of the pre-mRNA which is encoded by a gene selected from the group consisting of NEDD4L, MAP3K7, NFYA, ESYT2, MARK2, ST7, ARVCF, SYTL2, R3HDM1,COL4A3BP, TANGO2, SEPT9, ROBO1, FAM122B, CD47, LSR, PBX1,EPB41, ADAM15,EPB41L1, ABI1, FLNB,CTNND1, GPR160, ITGB3BP, INCENP, DENND1B, and CA12.

In some embodiments, the segment of the pre-mRNA is 9-150 nucleotides in length. In some embodiments, the composition comprises oligonucleotides. In some embodiments, the oligonucleotides are sufficiently complementary to the segment of the pre-mRNA. In some embodiments, the oligonucleotides have at least 80% sequence identity to the segment of the pre-mRNA. In some embodiments, the oligonucleotides have at least 90% sequence identity to the segment of the pre-mRNA. In some embodiments, the oligonucleotides comprise 10-50 nucleotides. In some embodiments, the oligonucleotides comprise 15-30 nucleotides. In some embodiments, the composition comprises small molecules, nucleic acid molecules, engineered cells, proteins, or a combination or modification thereof. In some embodiments, the composition comprises a chimeric molecule. In some embodiments, the chimeric molecule comprises a nucleic acid molecule and a protein. In some embodiments, the nucleic acid molecule comprises DNA, RNA, PNA, or a combination or hybrid thereof. In some embodiments, the composition induces or enhances exon skipping in the pre-mRNA. In some embodiments, the composition induces or enhances exon inclusion in the pre-mRNA. In some embodiments, the composition promotes a splicing switch in the pre-mRNA. In some embodiments, the composition down-regulates or up-regulates of splicing in the pre-mRNA. In some embodiments, the composition prevents splicing in the pre-mRNA.

Another aspect of the present disclosure provides a method for treating a disease or condition in a subject in need thereof, comprising: administering an effective amount of a composition to the subject, which composition specifically binds to a segment of a pre-mRNA which is encoded by a gene selected from the group consisting of NEDD4L, MAP3K7, NFYA, ESYT2, MARK2, ST7, ARVCF, SYTL2, R3HDM1, COL4A3BP, TANGO2, SEPT9, ROBO1, FAM122B, CD47, LSR, PBX1, EPB41, ADAM15, EPB41L1, ABI1, FLNB, CTNND1, GPR160, ITGB3BP, INCENP, DENND1B, and CA12, thereby modulating splicing in the pre-mRNA.

In some embodiments, the segment of the pre-mRNA is 9-150 nucleotides in length. In some embodiments, the composition comprises oligonucleotides. In some embodiments, the oligonucleotides are sufficiently complementary to the segment of the pre-mRNA. In some embodiments, the oligonucleotides have at least 80% sequence identity to the segment of the pre-mRNA. In some embodiments, the oligonucleotides have at least 90% sequence identity to the segment of the pre-mRNA. In some embodiments, the oligonucleotides comprise 10-50 nucleotides. In some embodiments, the oligonucleotides comprise 15-30 nucleotides. In some embodiments, the composition comprises small molecules, nucleic acid molecules, engineered cells, proteins, or a combination or modification thereof. In some embodiments, the composition comprises a chimeric molecule. In some embodiments, the chimeric molecule comprises a nucleic acid molecule and a protein. In some embodiments, the nucleic acid molecule comprises DNA, RNA, PNA, or a combination or hybrid thereof. In some embodiments, the composition induces or enhances exon skipping in the pre-mRNA. In some embodiments, the composition induces or enhances exon inclusion in the pre-mRNA. In some embodiments, the composition promotes a splicing switch in the pre-mRNA. In some embodiments, the composition down-regulates or up-regulates of splicing in the pre-mRNA. In some embodiments, the composition prevents splicing in the pre-mRNA. In some embodiments, the effective amount comprises at least 300 nM of the composition. In some embodiments, the effective amount comprises at most 500 nM of the composition. In some embodiments, the disease or condition comprises a genetic disease, a CNS disease, an inflammatory disease, a neurodegenerative disease, a cardiovascular disease, an autoimmune disease, or cancer. In some embodiments, the disease or condition is cancer. In some embodiments, the cancer comprises lung cancer, kidney cancer, or breast cancer. In some embodiments, the breast cancer is triple negative breast cancer.

Another aspect of the present disclosure provides a method for screening, diagnosis or prognosis of a disease or condition in a subject, comprising: (a) analyzing a biological sample from the subject to detect a level of expression of a protein isoform, which protein isoform is encoded by a gene selected from the group consisting of NEDD4L, MAP3K7, NFYA, ESYT2, MARK2, ST7, ARVCF, SYTL2, R3HDM1, COL4A3BP, TANGO2, SEPT9, ROBO1, FAM122B, CD47, LSR, PBX1, EPB41, ADAM15, EPB41L1, ABI1, FLNB, CTNND1, GPR160, ITGB3BP, INCENP, DENND1B, and CA12; and (b) determining a difference of the level of expression of the protein isoform in the biological sample relative to a level of expression of the protein isoform in a biological sample of a control, wherein the difference is indicative or predicative of the disease or condition.

In some embodiments, the biological sample is a cell, a tissue, or a blood sample. In some embodiments, (a) comprises quantitatively detecting an amount of the protein isoform in the biological sample. In some embodiments, the difference comprises an increase or a decrease of the level of expression of the protein isoform in the biological sample relative to the level of expression of the protein isoform in the biological sample of the control. In some embodiments, the increase of the level of expression of the protein isoform in the biological sample relative to the level of expression of the protein isoform in the biological sample of the control is indicative or predicative of the disease or condition. In some embodiments, the decrease of the level of expression of the protein isoform in the biological sample relative to the level of expression of the protein isoform in the biological sample of the control is indicative or predicative of the disease or condition. In some embodiments, the protein isoform comprises alternatively spliced protein isoforms. In some embodiments, the alternatively spliced protein isoforms are formed by alternative splicing of the gene. In some embodiments, the alternative splicing comprises exon skipping, exon inclusion, intron retention, competing 5′ splice sites, competing 3′ splice sites, multiple promoters, multiple poly(A) sites or a combination thereof. In some embodiments, the method further comprises detecting a level of expression of the gene in the biological sample. In some embodiments, the method further comprises detecting a presence or an absence of a difference of the level of expression of the gene in the biological sample of the subject relative to a level of expression of the gene in the biological sample in the control. In some embodiments, the presence or the absence of the difference of the level of expression of the gene is further indicative or predicative of the disease or condition. In some embodiments, the presence of the difference comprises an increase or a decrease of the level of expression of the gene in the biological sample of the subject relative to the level of expression of the gene in the biological sample in the control. In some embodiments, the method further comprises monitoring a progression of the disease or condition in the subject. In some embodiments, the monitoring comprises repeating (a) multiple times over a predetermined time period. In some embodiments, the method further comprises providing a treatment to the subject upon diagnosis of the disease or condition in the subject. In some embodiments, the treatment comprises administering to the subject an effective amount of a composition which modulates the level of the protein isoform expression. In some embodiments, the treatment comprises administering to the subject an effective amount of a composition which modulates splicing of the gene encoding the protein isoform. In some embodiments, the disease or condition comprises a genetic disease, a CNS disease, an inflammatory disease, a neurodegenerative disease, a cardiovascular disease, an autoimmune disease, or cancer. In some embodiments, the disease or condition is cancer. In some embodiments, the cancer comprises lung cancer, kidney cancer, or breast cancer. In some embodiments, the breast cancer is triple negative breast cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

This patent or application file contains at least one drawing executed in color. Copies of this patent or patent application publication with color drawing(s) will be provided by the Office upon request and payment of the necessary fee.

FIG. 1 shows an exemplary oncoprint summary of recurring genomic aberration and transcriptional changes for splicing associated RNA binding proteins in triple-negative breast cancer (TNBC) patient samples.

FIG. 2 schematically illustrates an example of luminal versus TNBC RNA-seq data analysis and target selection.

FIG. 3 shows an examplary diagram of splicing changes in the Cancer Genome Atlas (TCGA) and cell lines showing independent and overlapping events.

FIG. 4 shows examplary reverse transcription polymerase chain reaction (RT-PCR) images showing differential splicing of selected candidates in luminal versus TNBC cell lines.

FIG. 5 shows exemplary Western blot of protein lysates from luminal and basal cell lines showing the isoform expression at the protein level for different genes.

FIG. 6 shows a survival analysis of breast cancer patients expressing NEDD4L inclusion versus skipped isoforms showing poor overall survival for patents with skipped isoform.

FIG. 7 shows comparison of splicing differences and total gene expression differences across multiple breast cancer subtypes.

FIG. 8 illustrate SpliceLearn scores and corresponding eCLIP peaks around the NEDD4L exon trio. The scores can be used for designing oligo sequences and the bottom panel shows the splice switching experimental results in which the high scoring ASOs (GTGGGTTTCAGGGATTCTGA (SEQ ID NO: 1), CCCTGATTCAGACAGCAGGG (SEQ ID NO:2) significantly switched to the inclusion isoform in MDA-MB-231 cells.

FIG. 9 shows an exemplary experimental validation and quantitation of switching off NEDD4L in MCF7 and MDA-Mb-231 cells treated with 400 nM specific oligos and controls treated with either lipofectamine or PBS. Radioactive RT-PCR is shown above and quantitation of the results is shown below.

FIG. 10A shows an exemplary dose response curve for an SSO (GTGGGTTTCAGGGATTCTGA (SEQ ID NO: 1)) targeting NEDD4L which promotes inclusion. The SSO treatment causes dose dependent viability loss in MDA-Mb-231 cells compared to MCF7 cells. The optimal LC50 value is about 370 nM.

FIG. 10B shows an exemplary dose response curve for an SSO (CCCTGATTCAGACAGCAGGG (SEQ ID NO: 2)) targeting NEDD4L which promotes inclusion. SSO treatment causes dose dependent viability loss in MDA-Mb-231 cells compared to MCF7 cells. The optimal LC50 value is about 420 nM.

FIG. 11 shows exemplary PCR validations for a candidate gene.

FIG. 12 shows exemplary PCR validations for a candidate gene.

FIG. 13 shows exemplary PCR validations for a candidate gene.

FIG. 14 shows exemplary PCR validations for a candidate gene.

FIG. 15 shows an exemplary survival analysis of breast cancer patients expressing long and short isoforms for a candidate gene.

FIG. 16 shows an exemplary survival analysis of breast cancer patients expressing long and short isoforms for a candidate gene.

FIG. 17 shows an example selection criteria table.

FIG. 18 shows how the selection criteria is used to identify more target genes than use of splicing alone.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Identification of Candidate Targets for Therapeutic Development

In some aspects of the present disclosure, methods and systems for detecting or identifying candidate drug targets are provided. The candidate drug targets may be associated with specific diseases, illnesses or conditions. The diseases, illnesses or conditions may comprise cancer. Non-limiting examples of diseases, illnesses or conditions may include but not limited to ductal carcinoma in duct tissue in a mammary gland, medullary carcinomas, colloid carcinomas, tubular carcinomas, breast cancer or subtypes thereof; ovarian cancer, including epithelial ovarian tumors such as adenocarcinoma in the ovary and an adenocarcinoma that has migrated from the ovary into the abdominal cavity, uterine cancer, cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epithelioid carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system cancers (CNS) such as primary brain tumor, which includes gliomas (astrocytoma, anaplastic astrocytoma, or glioblastoma multiforme), Oligodendroglioma, Ependymoma, Meningioma, Lymphoma, Schwannoma, and Medulloblastoma; peripheral nervous system (PNS) cancers such as acoustic neuromas and malignant peripheral nerve sheath tumor (MPNST) including neurofibromas and schwannomas, malignant fibrous cytoma, malignant fibrous histiocytoma, malignant meningioma, malignant mesothelioma, and malignant mixed Mtillerian tumor; oral cavity and oropharyngeal cancer such as, hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer. In some embodiments, the pharmaceutical composition is for the treatment of a non-cancerous hyperproliferative disorder such as benign hyperplasia of the skin (e. g., psoriasis), restenosis, or prostate (e. g., benign prostatic hypertrophy (BPH)).

The candidate drug targets may comprise one or more genes that are differentially express, exons (e.g., exon duos or exon trios) that are differentially spliced, or a combination thereof. The methods and systems can be exon-centric and highly sensitive in detecting low-abundance aberrant mRNA isoforms.

Additionally, artificial intelligence (AI) may be utilized by the methods and systems as provided herein. The AI may comprise the use of machine learning algorithms, non-limiting examples of which may comprise supervised (or predictive) learning, semi-supervised learning, active learning, unsupervised machine learning, or reinforcement learning, support vector machines (SVM), linear, logistics, tress, random forest, xgboost, neural networks, deep neural networks, boosting techniques, bootstrapping techniques, ensemble techniques, or combinations thereof.

As provided herein, the systems or methods may comprise receiving data from a database. The database may be a public database (e.g., TCGA, GTEX, dbGAP), a private database, or a combination thereof. The database may comprise public data, proprietary data, or a combination thereof. The database may comprise clinical or biological data. The database may comprise RNA-seq data. The database may comprise data obtained from a variety of samples, e.g., greater than or equal to about 100, 200, 300, 400, 500, 600, 700, 800, 900, 1,000, 2,000, 3,000, 4,000, 5,000, 6,000, 7,000, 8,000, 9,000, 10,000, 15,000, 20,000, 25,000, 30,000, 40,000, 50,000, 60,000, 70,000, 80,000, 90,000, 100,000, 125,000, 150,000, 175,000, 200,000 samples, or more. At least a subset of the samples may be obtained from different subjects have the same or different diseases, illnesses or conditions.

The database may comprise data extracted or derived from samples from cell lines from certain diseases, illnesses or conditions, and/or from subjects having certain different diseases, illnesses or conditions. In some cases, the diseases, illnesses or conditions comprise breast cancer or subtypes thereof, for example, liminal A, luminal B, Her2+, TNBC. Non-limiting examples of cell lines may comprise BT483, CAMA1, EFM19, HCC1428, HCC712, IBEP2, KPL1, LY2, MCF7, MDAMB 134, MDAMB134V1, MDAMB 157, MDAMB 175, MDAMB175VII, MDAMB231, MDAMB330, MDAMB361, MDAMB415, MDAMB435, MDAMB436, MDAMB453, MDAMB468, T47D, ZR751, ZR75B, BSMZ, BT474, EFM192A,IBEP1, IBEP3, UACC812, ZR7527, ZR7530, 21MT1, 21MT2, 21NT, 21PT, AU565, HCC1008, HCC1569, HCC1954, HCC202, HCC2218, HH315, HH375, KPL-4, OCUB-F, SKBR3, SKBRS, SUM19OPT, SUM225CWN, UACC893, BT20, CAL148, DU4475, EMG3, HCC38, HCC1143,HCC1187, HCC1395, HCC1599, HCC1739, HCC1806, HCC1937, HCC2157, HCC3153, HCC70, HMT3522, KPL-3C, MA11,MFM223, SUM185PE, SUM229PE, BT549, CAL120, CAL851, HDQ-P1, Hs578T, SKBR7, SUM102PT, SUM1315M02, SUM149PT, SUM159PT, or any combination thereof.

The data may be subject to one or more analysis or processing steps. The data may be analyzed and/or quantified to identify information or event(s) such as a splicing event. The information or event(s) identified may be statistically significant or specific to one or more diseases, illnesses or conditions. The data analysis or processing may comprise mapping the data to genomes, transcriptomes, or a combination thereof. The data may be processed to remove any information that may not be related to genomes, transcriptomes, or a combination thereof. The data may be processed or analyzed based on one or more predetermined parameters or criteria including, such as types or subtypes of diseases, illnesses, or conditions.

In cases where a database comprises data associated with different types or subtypes of diseases, illnesses, or conditions, data related to each type or subtype may be analyzed or processed individually to identify information or an event(s) that may be statistically significant or specific to each type or subtype. The identified information or event(s) may be grouped together and compared to one or more controls to determine one or more candidate targets. Alternatively, the information or event(s) identified for each subtype or type may be compared with one another to generate a list of candidate targets. In some cases, the candidate targets comprise information or an event(s) that is identified or shared by at least two different types or subtypes.

The list of candidate targets may comprise any number of candidate targets, for example, greater than or equal to about 5, 10, 20, 30, 40, 50, 60, 70, 80, 90, 100, 120, 140, 160, 180, 200, 220, 240, 260, 280, 300, 350, 400, 450, 500, or more. In some cases, the list may comprise a number of candidate targets falling between any of the two values described above, for example, about 275.

At least a portion (e.g., at least about 10%, 20%, 30%, 40%, 50%, 60%, 70%, 80%, 90%, or more) of the candidate targets generated may be subjected to further data analysis or processing. The candidate targets may be arranged in certain order based upon one or more parameters or criteria. The candidate targets may be selected based upon one or more parameters or criteria, thereby generating a refined list of candidate targets. Non-limiting examples of the parameters which may be used to arrange the candidate targets comprise a splicing index, a disease index, a splice-switching oligonucleotides (SSO) druggability index, or any combination thereof.

The splicing index may be determined based at least in part on factors including e.g., splicing change in a sample (or data) analyzed as compared to a control, consistency and/or reproducibility of a given information or event(s) (e.g., in patient dataset(s)), recurrence of a given information or event(s) in multiple disease datasets, an absence of a given information or event in a normal or control dataset(s). Each factor may be given the same or a different weight in the determination and based on the determination, a score may be generated.

The disease index may be determined based at least in part on factors including e.g., an impact of a given information or event(s) such as a splice change on function of an expression product(s) such as a protein(s), the degree of association with a disease, illness, or condition, pathway analysis such as pathways listed in kyoto encyclopedia of genes and genomes (KEGG) database, literature evidence, or any combination thereof. Each factor may be given the same or a different weight in the determination and based on the determination, a score may be generated.

The SSO druggability index may be determined based at least in part on factors including e.g., an ability to identify unique and/or specific splice correcting molecules (e.g., oligo sequence(s)) using AI such as machine learning algorithms, a presence or absence of a strong enhanced cros slinking and immunoprecipitation (eCLIP) peak(s) mapped to the identified target (e.g., an exon(s)), a presence or absence of a disease specific expression of an isoform(s) as compared to the genotype-tissue expression (GTEx) normal data, or any combination thereof. Each factor may be given the same or a different weight in the determination and a score may be generated based on the determination.

In cases in which a refined list of candidate targets is generated, candidate targets comprised in the list may be subject to additional analysis or processing steps. For example, splicing events associated with at least a subset of the candidate targets may be subjected to an evaluation process. The evaluation process may evaluate for expression at various levels, for example, at the RNA level, at a protein level, or both. The evaluation process may confirm differential isoform expression in samples from different diseases (or subtypes thereof), illnesses, or conditions. Upon confirmation, the candidate targets may be selected and used for further development of therapeutics. In some cases, the selected targets comprise one or more genes. Non-limiting examples of the one or more genes may comprise NEDD4L (ENV2), MAP3K7 (ENV3), NFYA (ENV11), ESYT2 (ENV21), MARK2 (ENV18), ST7 (ENV19), ARVCF (ENV22), SYTL2 (ENV17), R3HDM1 (ENV23), COL4A3BP (ENV9), TANGO2 (ENV6), SEPT9 (ENV15), ROBO1 (ENV4), FAM122B (ENV5), CD47 (ENV13), LSR (ENV20), PBX1 (ENV16), EPB41 (ENV14), ADAM15 (ENV7), EPB41L1 (ENV8), ABI1 (ENV10), FLNB (ENV1), CTNND1 (ENV12), GPR160 (ENV24), ITGB3BP (ENV25), INCENP (ENV26), DENND1B (ENV27), CA12 (ENV28), or any combination thereof.

Modulation of Targets using various Modalities

In some aspects of the present disclosure, methods and systems for modulating a splicing target(s) are provided. The modulation may comprise modulating a splicing event(s) associated with a target (e.g., a gene). The modulation may comprise promoting or facilitating a splice switching. For example, the modulation may comprise switching pathogenic isoforms to non-pathogenic isoforms.

The modulation may comprise the use of one or more compositions or molecules which may interact specifically with (e.g., hybridize) a target so as to control or alter splicing of the target or regulate expression of the target at the RNA level, protein level or both. The compositions or molecules may be targeted to any element or combination of elements (e.g., one or more genomic regions within a target) that regulate splicing, including such as the 3 ‘splice site, the 5’ splice site, the branch point, the polypyrimidine tract, exonic splicing enhancers, exonic splicing silencers, intronic splicing enhancers, intronic splicing silencers, or any combination thereof.

The compositions or molecules may comprise e.g., small molecules, polymers (natural or synthetic), nucleotide sequences such as oligonucleotides or RNAs, a therapeutic agent(s), cells such as CAR-T cells, a protein such as an antibody, or any combination thereof.

The compositions or molecules may be admixed, encapsulated, conjugated, or otherwise associated with other molecules, molecule structures, or mixtures of compounds, for example liposomes, receptor targeted molecules, oral, rectal, topical or other formulation, for assisting in uptake, distribution, and/or absorption.

The compositions or molecules may be applied in vivo or ex vivo. To achieve target-specific, or disease-specific targeting, the compositions or molecules may be added at a certain concentration. For example, the compositions or molecules may have a concentration that is less than or equal to about 5 micromolar (μM), 4 μM, 3μM, 2 μM, 1 μM, 900 nanomolar (nM), 800 nM, 700 nM, 650 nM, 600 nM, 550 nM, 500 nM, 450 nM, 400 nM, 350 nM, 300 nM, 250 nM, 200 nM, 150 nM, 100 nM, 50 nM, 10 nM, or less. The concentration may be greater than or equal to about 1 nM, 10 nM, 50 nM, 100 nM, 200 nM, 300 nM, 400 nM, 500 nM, 600 nM, 700 nM, 800 nM, 900 nM, or more. In some cases, the concentration may fall between any two of the values discussed above, for example, about 370 nM or 420 nM.

In some cases, the compositions or molecules comprise oligonucleotides. The oligonucleotides may comprise any number of nucleotides or nucleotide residues, for example, greater than or equal to about 5, 10, 12, 14, 16, 18, 20, 22, 24, 26, 28, 30, 32, 34, 36, 38, 40, 42, 44, 46, 48, 50, 55, 60 nucleotides or nucleotide residues, or more. In some cases, the oligonucleotides may comprise less than or equal to about 50, 45, 40, 35, 30, 29, 27, 25, 24, 23, 22, 21, 19, 18, 17, 16, 13, 10, 8, 6 nucleotides or nucleotide residues, or less. In some cases, the number of nucleotides or nucleotide residues comprised in the oligonucleotides may fall between any of the values described above, for example, about 16 (16-mer), 17 (17-mer), 18 (18-mer), 19 (19-mer), 20 (20-mer), 21 (16-mer), or 22 (22-mer). In some cases, the oligonucleotides comprise DNA molecules, RNA molecules, or a combination thereof.

In some cases, the oligonucleotides comprise antisense oligonucleotides. The antisense oligonucleotides may be DNA and/or RNA oligos which are complementary to a given sequence, which given sequence may be a region within a target gene.

The oligonucleotides may be prepared using various technologies such as solid phase synthesis, the phosphorothioates and/or alkylated derivatives. The nucleotides or nucleotide residues comprised in the oligonucleotides may comprise natural, unmodified nucleotides (e.g., cytosine, guanine, adenine, uracil or thymidine), modified nucleotides, or any combination thereof. In some cases, modified nucleotides or bases are used. The modification may be designed to enhance binding affinity. The modification may comprise chemical modifications. The modification may comprise backbone modifications, sugar ring modifications, or a combination thereof. The sugar ring modifications may comprise 2′-sugar modifications. As an example, an oligonucleotide of the present disclosure may comprise a phosphothioate-modified backbone and/or ribose sugar modified to contain methoxy ethane at 2′-position (2′MOE). The oligonucleotides comprising modified nucleotides or bases may not activate RNase H. In some cases, one or more (e.g., at least about 1, 2, 3, 4, 5, 6, 7, 8, 9, 10, 12, 14, 16, 18, 20, 22, 24, 26, 28, 30, 35, 40, 45, or more) of the inter-nucleotide bridging phosphate residues are modified phosphates, such as methyl phosphonates, methyl phosphonothioates, phosphoromorpholidates, phosphoropiperazidates, phosphoroamidates, or any combination thereof. In some cases, one or more (e.g., at least about 1, 2, 3, 4, 5, 6, 7, 8, 9, 10, 12, 14, 16, 18, 20, 22, 24, 26, 28, 30, 35, 40, 45, or more) of the nucleotides or bases comprise a 2′-alkyl moiety (e.g., C1-C4 alkyl, linear or branched, saturated or unsaturated including e.g., methyl, ethyl, ethenyl, propyl, 1-propenyl, 2-propenyl, isopropyl, or combination or derivative thereof).

In some cases, the compositions or molecules comprise small molecules. The small molecules may comprise a broad range of chemical compounds that can switch the isoforms of the above-mentioned targets either at the pre-mRNA level or protein level. These compounds may be identified through high-throughput screening approaches using chemical libraries, wherein addition of a compound or a combination of compounds can induce an isoform switch or modulate (e.g., inhibit or enhance) the biological activity of a specific isoform of one or several of the genes (e.g., genes mentioned above or described elsewhere herein) either at the level of RNA or protein or both.

### Application

The systems and methods of the present disclosure can be used for determining or identifying a novel splicing event(s) or a target (e.g., a gene) to which the novel splicing event is associated with. The novel splicing event(s) may be statistically significant or specific to one or more given types or subtypes of diseases, illnesses or conditions. To identify the novel splicing events, the methods and systems of the present disclosure may receive data from one or more databases, public and/or private, which data may comprise biologically relevant data with respect to the types or subtypes of diseases, illnesses or conditions which are under investigation. The data may be analyzed, processed or annotated. The data analysis, processing, and/or annotation may be conducted using machine learning algorithms. The machine learning may be a supervised learning, an unsupervised learning, or a combination thereof. The algorithm may be a trained algorithm. The algorithm may be trained using a training set. The training set may comprise training samples. The training samples may be cell lines from certain diseases, illnesses, or conditions; samples obtained from subjects having certain diseases, illnesses, or conditions; controls including positive and/or negative controls; or any combination thereof.

The data analysis, processing, and/or annotation may generate a list of candidate targets which may potentially be used for therapeutic development. Candidate targets comprised in the list may be subjected to further screening process or analyses. Splicing of the candidate targets may be evaluated or validated. The evaluation or validation of the candidate targets may yield a refined list of targets which may be subjected to further therapeutic development.

Splicing of individual targets comprised in the refined list may be using compositions or molecules. The splicing may be modulated to promote switching of pathogenic isoforms to non-pathogenic isoforms. The compositions or molecules may be designed to target a select region or a combination of select regions within a target to achieve a disease-specific targeting. In some cases, the compositions or molecules are designed to modulate the splicing of two or more (e.g., at least 3 ,4, 5, 6, 7, 8, 9, 10, 11, 12, 13, 14, 15, or more) different targets. Compositions or molecules targeting different targets may be used sequentially, or simultaneously.

In some aspects of the present disclosure, methods and systems for providing treatment to a subject having or suspected to have a disease, illness, or condition are provided. The methods and systems may comprise obtaining a sample from the subject having or suspected to have a disease, illness, or condition. Biologically relevant data (e.g., DNA, RNA-seq data) may be derived or extracted from the sample. The biologically relevant data may be screened or processed to remove any data unrelated to genome(s) or transcriptome(s). The processed data may be subjected to one or more data analysis, processing or annotation processes which may identify one or more novel splicing events statistically significant or specific to the disease, illness, or condition the subject has or suspected to have. The splicing events identified may be further analyzed or filtered using one or more parameters or filtering criteria, which may generate a final list of splicing events and targets associated therewith for the treatment.

Upon identification of the targets, the systems and methods may further comprise administering a therapeutically effective amount of compositions or molecules to the subject having or suspected to have the disease, illness, or condition. The administration may be conducted within a given time period. The subject may be monitored, and the amount of the compositions or molecules administered to the subject may be adjusted depending upon, the monitoring results. Additionally, the monitoring may comprise obtaining one or more samples from the subject while the subject is under treatment. The one or more samples may be analyzed or tested to determine if the treatment is effective or not. If a treatment is determined to be ineffective, the treatment may be ceased and/or a different treatment (e.g., administering a different type of compositions or molecules) may be provided.

### EXAMPLES

### Example 1

### Identification of Alternatively Spliced Transcripts in Triple Negative Breast Cancer (TNBC) and Therapeutics to Correct the Splicing Change

Breast cancer is the most commonly diagnosed cancer and the second leading cause of cancer mortality in women, with nearly 30% of primary disease diagnoses that result in metastatic breast cancer. One of the challenges in breast cancer treatment is to overcome its large heterogeneity and distinct cancer subtypes that may demand differential treatments including chemotherapy, hormonal therapy, and human epidermal growth factor receptor 2 (Her2-) targeted therapy (depending on the subtype). However, a significant number of patients may develop resistance to current standard of care therapies. This stresses the need for identification of novel targets and development of alternative therapies for complete disease remission.

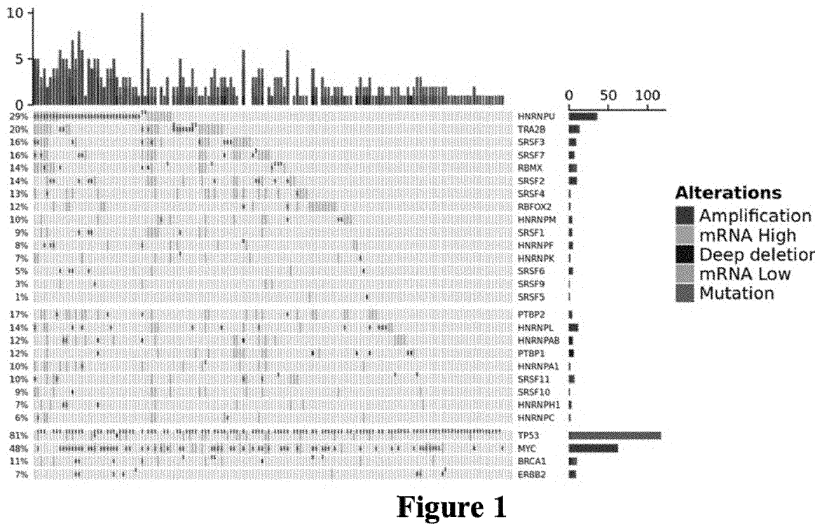

Splicing errors can be a source of coding variation in breast cancer. Aberrant splicing of several genes may occur even without DNA mutations or epigenetic changes due to mis-regulated expression of splicing factors in breast cancer. Multiple studies have indicated the oncogenic role of core splicing factors such as SRSF1, SRS F2, ESRP1, RBFOX1, etc. in breast cancer. For example, overexpression of SRSF1 may promote transformation of non-cancerous breast epithelial cells. Additionally, it has been suggested that spliceosomal components may be particularly essential factors in TNBC subtype, and the TNBC tumors sometimes show dependency on these factors. As an example, FIG. 1 shows an oncoprint summary of recurring genomic aberration and transcriptional changes for splicing associated RNA binding proteins in the Cancer Genome Atlas (TCGA) breast cancer TNBC subtype patient samples.

Additionally, splice site mutations that result in expression of alternative isoforms have been reported in key breast cancer genes such as ESR1 encoding estrogen receptor alpha rendering resistance to first line drugs such as Tamoxifen in ER positive breast cancers. Mis-regulation of splicing factors have been shown to contribute to epithelial to mesenchymal switch by the production of mesenchymal isoforms of critical genes such as CD44 and FGFR2, thereby promoting tumor progression and metastasis. Given the suboptimal treatment options available for TNBC patients and the widespread splicing errors observed in TNBC patient RNA-seq data, disease specific alternative splicing can be a source of actionable candidates that can be therapeutically targeted.

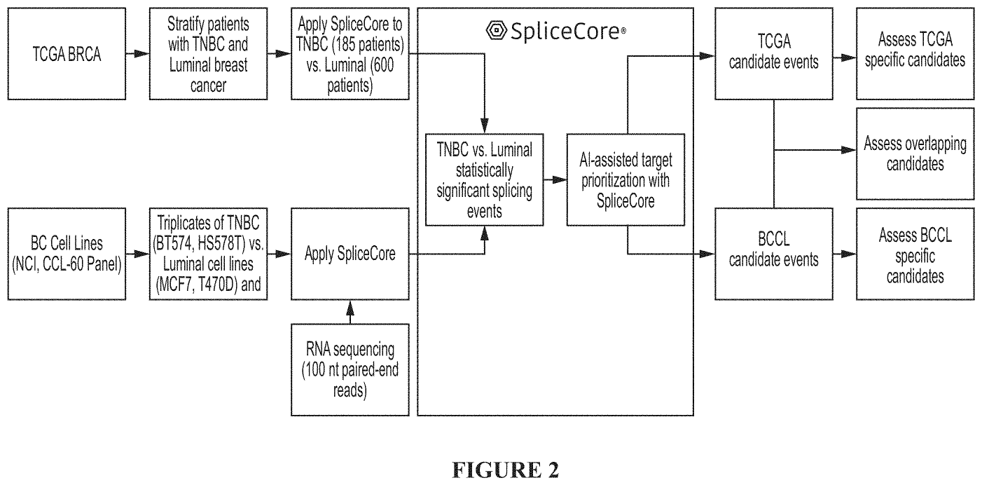

Systems of the present disclosure can be used to discover recurrent splicing changes in TNBC patient RNA-seq and design modalities such as antisense oligonucleotides that target the specific isoforms in order to promote splice-switching to achieve a therapeutic benefit. As discussed above or elsewhere herein, the systems may comprise the SpliceCore® software platform described in International Patent Application No. WO2019/226804, which has been incorporated herein by reference in its entirety. In order to discover splicing changes that occur in TNBC subtypes, RNA-seq data from luminal subtype patients and TNBC subtype patients from TCGA breast cancer datasets are compared using the SpliceCore® software platform. In addition, RNA-sequencing in triplicates on breast cancer cell lines is performed. Two representative luminal cell lines (i.e., MCF7, T47D) and two representative TNBC B subtype cell lines (i.e., HS578T, BT549) and one TNBC A subtype cell line (i.e., MDA-MB 468) are used. A flowchart of the SpliceCore® analysis of the TCGA and the cell line RNA seq data is illustrated in FIG. 2.

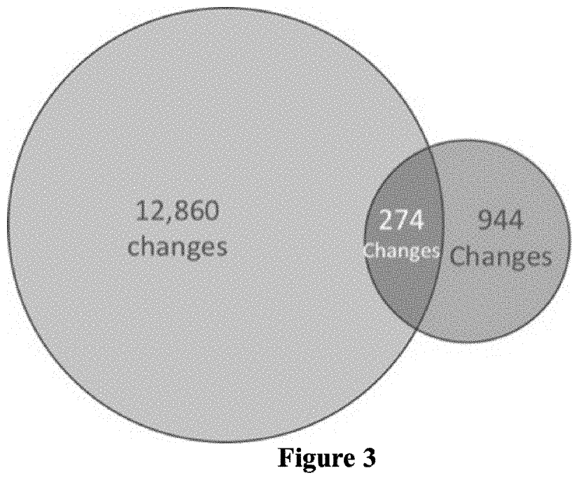

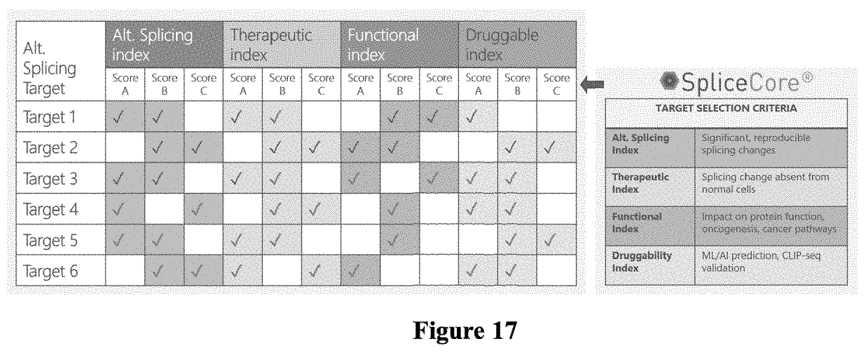

Comparison of the splicing changes between luminal breast cancer and basal breast cancer from TCGA and the cell lines may independently result in an identification of splicing changes that are distinct to TCGA (12,860 changes) and changes that are distinct to cell lines (944 changes) (FIG. 3). Among those changes, there are about 274 splicing changes that are identified in both TCGA and cell lines (FIG. 3). These 274 splicing changes are considered candidates for target selection and may be subject to further analysis. The analysis may be prioritized based on a number of parameters. The parameters (or buckets) for candidate selection may serve as diversified target selection criteria, which may include e.g., a splicing index, a disease index, a therapeutic index, a functional index, a splice-switching oligonucleotide (SSO) druggability index or any combination thereof. Example selection criteria and results are shown in FIG. 17. The figure shows a representative illustration of candidate selection scoring matrix based on different parameters described above.

The splicing index can score for the splicing change (dPSI) in a given case and a control, consistency, reproducibility of a given event in patient datasets, and recurrence of a given event in multiple disease datasets and absent in normal datasets. The disease index can score for impact of the splicing change on protein function (i.e., “Splicelmpact™”), disease association (integrated through Open Target scores), pathway analysis (KEGG), and literature evidence. The SSO druggability index can score for the ability to identify unique and specific splice correcting oligo sequence using machine learning (i.e., “SpliceLearn™ scores”), presence of strong eCLIP peaks mapped to the identified exons, and disease specific expression of the isoform (comparison with GTex normal data).

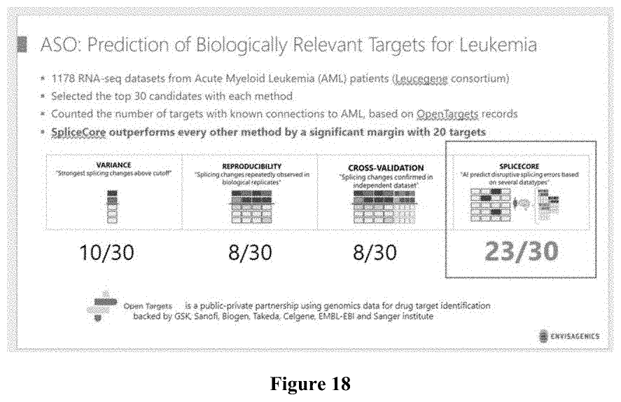

FIG. 18 reveals the identification of biologically relevant targets for the treatment of leukemia as described herein. About 1178 RNA-seq datasets from Acute Myeloid Leukemia (AML) patients obtained from the Leucegene consortium were analyzed to identify potential therapeutic targets. Different approaches were used to analyze alternative splicing events including variance assessment (selection of strongest splicing changes above a cutoff), reproducibility (selection for splicing changes repeatedly observed in biological replicates), cross-validation (splicing changes confirmed in independent dataset(s)), and SpliceCore® (software platform described in International Patent Application No. WO2019/226804, which has been incorporated herein by reference in its entirety). For each approach, the top 30 gene candidates were selected and the gene candidates that were also known to be connected to AML pathogenesis using records from OpenTargets (a public-private partnership using genomics data for drug target identification backed by GSK, Sanofi, Biogen, Takeda, Celgene, EMBL-EBI and Sanger Institute). As shown in FIG. 18, 23 of the top 30 candidates identified by SpliceCore were known to be connected to AML. The other approaches identified few candidates known to be connected to AML: the variance approach identified 10 targets; the reproducibility approach identified 8 targets; and the cros 5-validation approach identified 8 targets.

In an exemplary application of the selection criteria in FIG. 17, the alternative splicing index is determined by observing one or more alternative splicing event(s)/change(s) in in-house cell lines and public BRCA TCGA RNA-seq data; the therapeutic index is determined by confirming that the one or more alternative splicing event(s)/change(s) is/are disease-specific and is/are not found in normal breast tissues using public GTEx RNA-seq; the functional index is determined by noting that the score(s) for the one or more alternative splicing event(s)/change(s) generated by Splicelmpact (software platform described in International Patent Application No. WO2019/226804, which has been incorporated herein by reference in its entirety) are significantly disruptive; and the druggable index is determined by using SpliceLearn (software platform described in International Patent Application No. WO2019/226804, which has been incorporated herein by reference in its entirety) to predict that the one or more alternative splicing event(s)/change(s) is a drug target. In some cases, the drug target is an SSO modulatory target.

In an another exemplary application of the selection criteria in FIG. 17, the alternative splicing index is determined by observing one or more alternative splicing event(s)/change(s) in in-house organoids and public BRCA TCGA RNA-seq from the Metabrick dataset; the therapeutic index is determined by confirming that the one or more alternative splicing event(s)/change(s) is/are disease-specific and is/are not found in various post-mortem tissues including liver, heart, muscle and/or kidney, using public GTEx RNA-seq; the functional index is determined by noting that the one or more alternative splicing event(s)/change(s) occur in one or more genes with high BRCA-association scores estimated using OpenTargets; and the druggable index is determined by confirming that binding of one or more oncogenic splicing factors to the one or more target genes with the one or more alternative splicing event(s)/change(s) using CLIP-seq data. In some cases, the one or more target genes may be blocked by ASO based in the selection criteria analyses.

In an another exemplary application of the selection criteria in FIG. 17, the alternative splicing index is determined by observing one or more alternative splicing event(s)/change(s) in in-house cell lines and licensed RNA-seq from a partner; the therapeutic index is determined by confirming that the one or more alternative splicing event(s)/change(s) is/are disease-specific and is/are not found in normal tissues from partner RNA-seq; the functional index is determined by confirming that the one or more alternative splicing event(s)/change(s) are functionally related breast cancer using public literature; and the druggable index is determined by confirming that one or more alternative splicing event(s)/change(s) occurs in one or more genes that is/are known to be small molecule protein target(s).

Based on one or more of the above-mentioned filtering criteria or parameters, a total of 28 candidates whose splice changes may be significant in TNBC subtype (Table 1—List of TNBC specific top scoring splicing events and their corresponding gene names) are selected. These candidates' splicing events are subsequently subjected to an evaluation for expression at the level of RNA through PCR in experimental models such as cell lines, primary cells, tissues, organoids, PDX tumors, patient tissue material, body fluids, etc. Wherever antibodies are available, splicing isoforms may also be evaluated for protein expression. Hybridization methods such as RNA-FISH can also be used to validate the specific isoform expression in tumor tissue sections.

TABLE 1

| | Gene | ENV |
|---|---|---|
| TXDBID | (HUGO) | Code |
| CA-18-58335477-58335537.2267.0 | NEDD4L | ENV2 |
| CA-6-90544551-90544632.1 | MAP3K7 | ENV3 |
| CA-6-41080810-41080897.1 | NFYA | ENV11 |
| CA-7-158752780-158752843.1 | ESYT2 | ENV21 |
| CA-11-63903985-63904147.1 | MARK2 | ENV18 |
| CA-7-117134123-117134192.1 | ST7 | ENV19 |
| CA-22-19971215-19971335.1 | ARVCF | ENV22 |
| CA-11-85717482-85717530.1 | SYTL2 | ENV17 |
| CA-2-135641535-135641604.2 | R3HDM1 | ENV23 |
| CA-5-75399309-75399387.1 | COL4A3BP | ENV9 |
| CA-22-20053436-20053551.3 | TANGO2 | ENV6 |
| CA-17-77307140-77307197.5 | SEPT9 | ENV15 |
| CA-3-78647628-78647655.1 | ROBO1 | ENV4 |
| CA-X-134772142-134772283.1 | FAM122B | ENV5 |
| CA-3-58141857-58141929.1 | FLNB | ENV1 |
| CA-3-108050577-108050602.2 | CD47 | ENV13 |
| CA-1-29058588-29058645.2 | EPB41 | ENV14 |
| CA-1-164820071-164820184.1 | PBX1 | ENV16 |
| CA-19-35262545-35262692.1 | LSR | ENV20 |
| CA-1-155061417-155061489.4 | ADAM15 | ENV7 |
| CA-20-36209487-36209898.2 | EPB41L1 | ENV8 |
| CA-10-26755654-26755741.1 | ABI1 | ENV10 |
| CA-11-57791493-57791673.2 | CTNND1 | ENV12 |
| CA-3-170082616-170082758.1 | GPR160 | ENV24 |
| CA-1-63447564-63447614.1 | ITGB3BP | ENV25 |
| CA-11-62141499-62141511.1 | INCENP | ENV26 |
| CA-1-197647054-197647114.1 | DENND1B | ENV27 |
| CA-15-63328097-63328130.1 | CA12 | ENV28 |

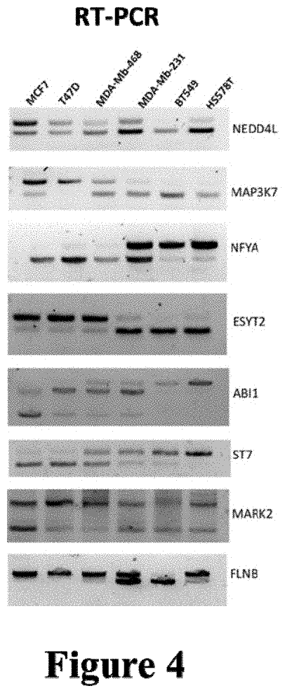

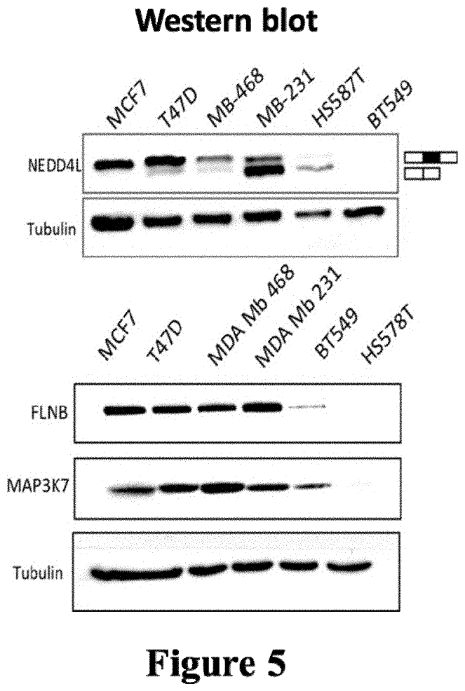

First, reverse transcription polymerase chain reaction (RT-PCR) is performed on selected candidates from the list of Table 1 to confirm the differential isoform expression in luminal versus the basal cell lines. Representative PCRs are shown in FIG. 4 where specific expression of the long or the short isoform is enriched in TNBC cell lines versus luminal cell lines. Further, for a select group of candidates, western blot analysis is also performed to verify the protein expression, and the representative western blot images for those candidates are shown in FIG. 5, which also shows differential isoform expression in protein lysates extracted from luminal and basal cell lines.

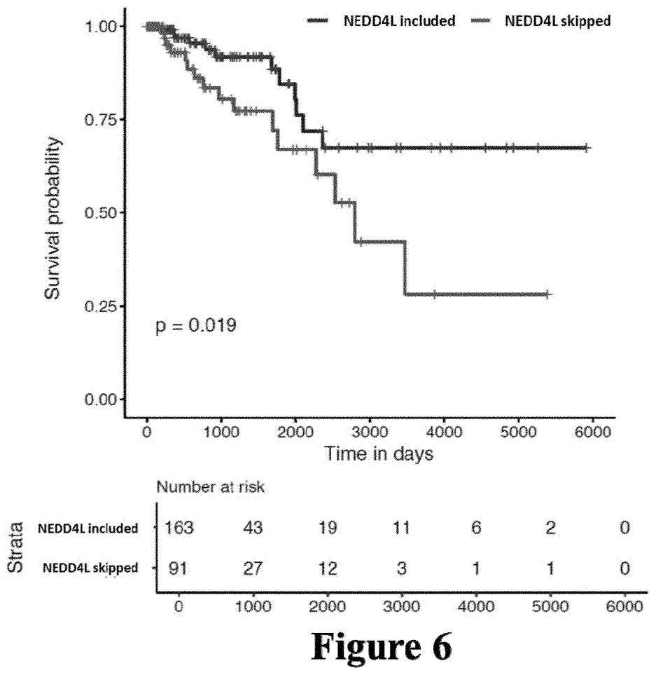

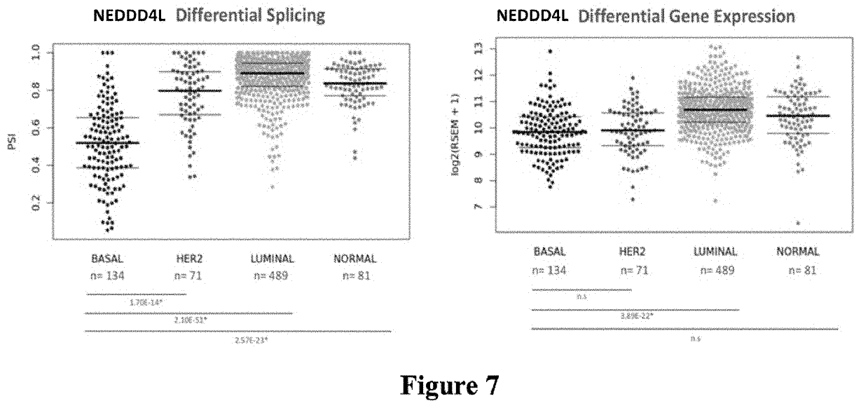

Next, specific candidates are focused on to test to see if they can be used as a good therapeutic target for the development therapeutic compounds. NEDD4L is suggested by the SpliceCore software platform as one of the top candidates and has shown the strongest dPSI change in TCGA data and very high reproducibility, along with known cancer association in a key signaling pathway (i.e., TGFbeta). By studying the impact of the observed splicing changes on protein function, it is determined that the skipping isoform (short isoform) enriched in TNBC subtype lacks a short loop region next to the WW domain which may be responsible for protein-protein interaction. The loop contains a Threonine residue that may undergo post-translational modification by a kinase, which can phosphorylate NEDD4L to maintain the homeostasis of TGFbeta signaling. The TNBC cancer-specific loss of the loop through alternative splicing may deregulate the signaling cascade leading to tumor progression. Additionally, it is observed that breast cancer patients expressing the inclusion isoform of NEDD4L have a better overall survival compared to the breast cancer patients that have the expression of the skipped isoform (FIG. 6). Further analyses of the subtype stratified data from TCGA have shown that the NEDD4L-skipping isoform is significantly enriched in TNBC subtype compared to normal breast or luminal or HER2 subtypes of breast cancer. This difference has been observed only at the level of alternative splicing and there is only a modest difference in the total RNA expression of NEDD4L across these subtypes (FIG. 7).

By using the SpliceCore® software platform and a module called SpliceLearn, a machine-learning-based (ML-based) approach is used to predict nucleotide sequences with high likelihoods of promoting a splicing switch if blocked using a molecule (e.g., an oligonucleotide). These sequences are scored, and rank-ordered for every exon trio on the candidate list. Additional scoring criteria may include the RBP binding peaks which can be obtained from ENCODE eCLIP-seq data and/or in-house generated eCLIP-seq data for splicing regulatory proteins including but not limited to RBFOX2, TDP-43, HNRNPL.

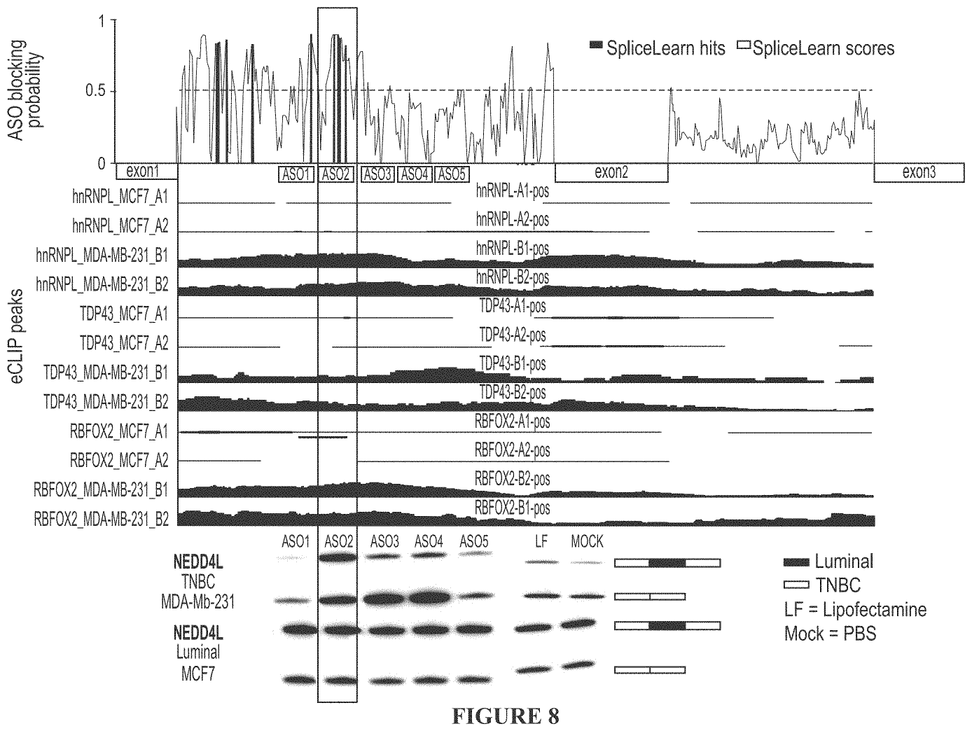

Next, a list of k-mer sequences can be generated that span across the high scoring regions within the exon trio and may further be filtered based on SSO specificity, repeat motifs, and off-target effects, and secondary structure (FIG. 8). The top 5 oligos are chemically synthesized and may contain phosphothioate-modified backbone and/or ribose sugar uniformly modified to contain methoxy ethane in 2′ position (2′MOE). Purified oligonucleotides can be subjected to functional assays in breast cancer cell lines.

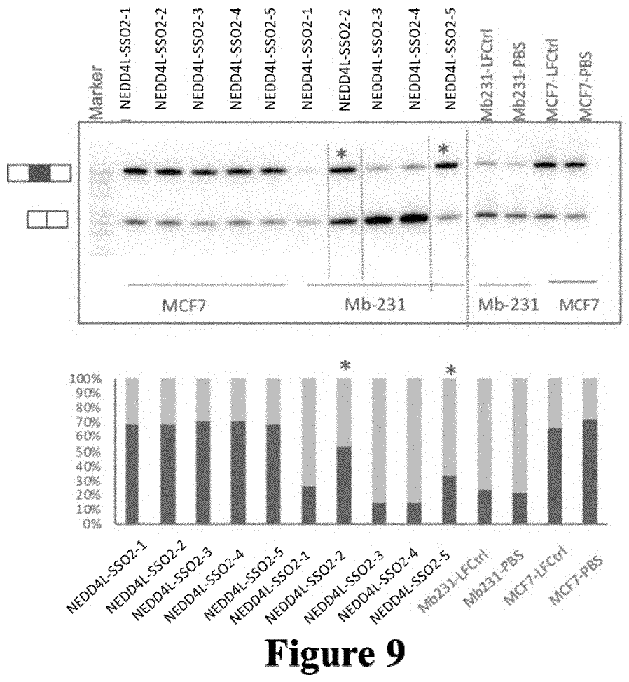

For NEDD4L, 5 different sequences (GCTGGCTTTGTCTGGATAGG (SEQ ID NO: 3), GTGGGTTTCAGGGATTCTGA (SEQ ID NO: 1), TCTCACGTCACCTGCCTTAC (SEQ ID NO: 4), AGCGCTGCCACAGCAGTGGG (SEQ ID NO: 5),CCCTGATTCAGACAGCAGGG (SEQ ID NO: 2)) are tested in total, and 2 of those sequences are found to promote the inclusion of the middle exon significantly. GTGGGTTTCAGGGATTCTGA (SEQ ID NO: 1) (SSO2-2) is shown to promote an average of 40% inclusion ratio in 3 out of 4 experiments, and (SSO2-5) CCCTGATTCAGACAGCAGGG (SEQ ID NO: 2) is shown to promote an average of 30% inclusion ratio in 4 out of 4 experiments (FIG. 9).

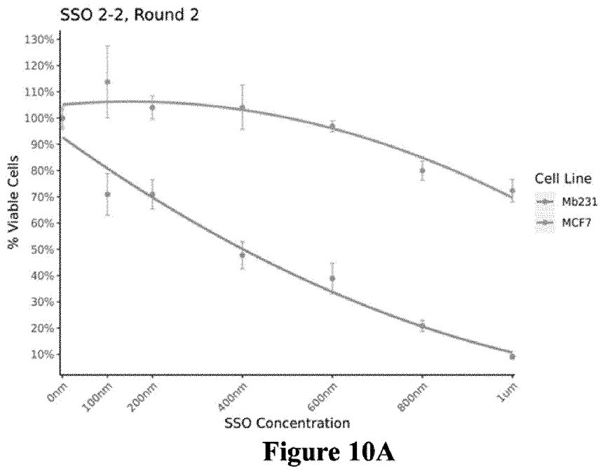

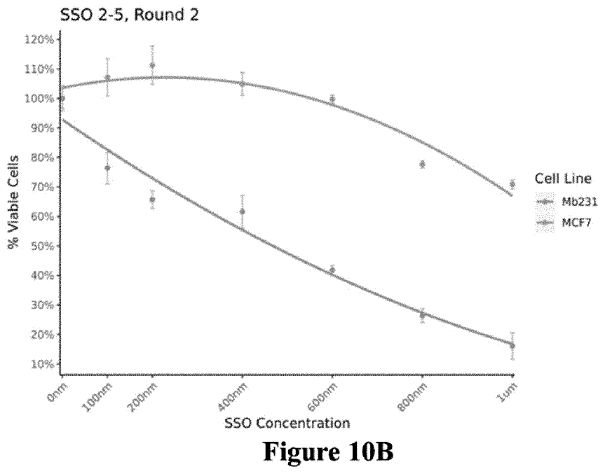

Dosage response experiments are performed to evaluate the LC50 value for the SSO compounds in 2 breast cancer cell lines—i.e., the MCF7 (luminal) and MDA-MB-231 (TNBC) cell lines. Transfection of the SSO compounds show a dose responsive loss of viability in the MDA-MB-231 cells and not in the MCF7 cells. The optimal dosing concentration in cell lines is found to be between 370 nM-420 nM where >50% of the cell death has been observed. The cell viability can be evaluated by measuring the mitochondrial ATP flux using the Celltitre glow assay. The mean luminescence can be converted to percentage of viable cells after normalizing to control untransfected cells. The dose response curve for both the SSO on two different cell lines is shown in FIGS. 10A-10B.

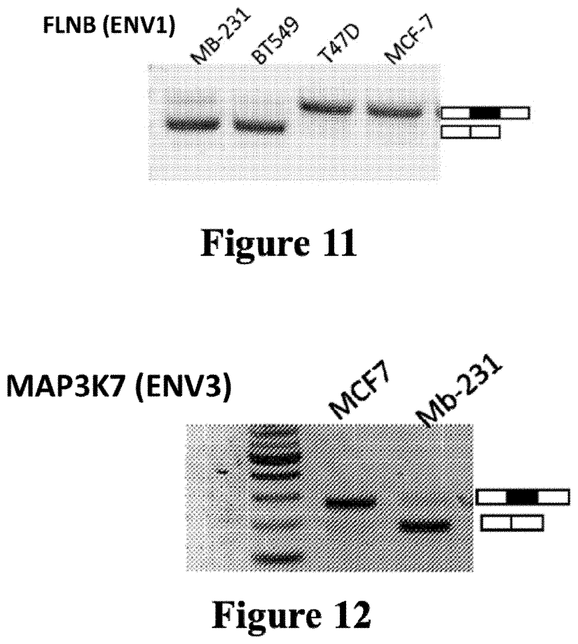

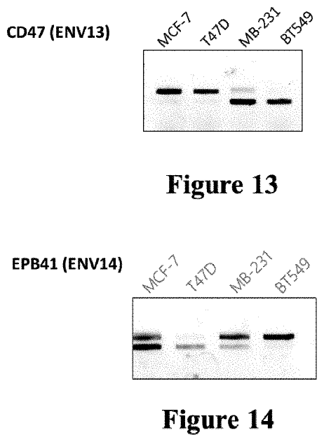

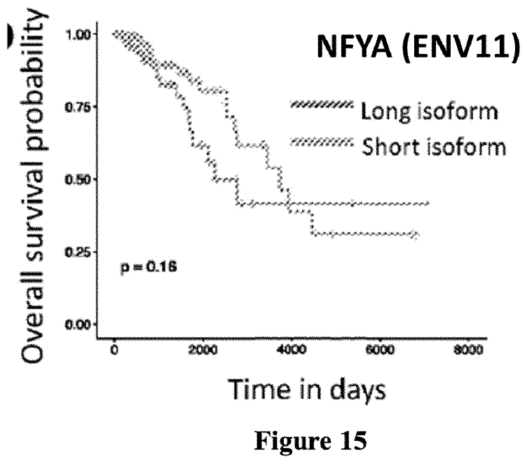

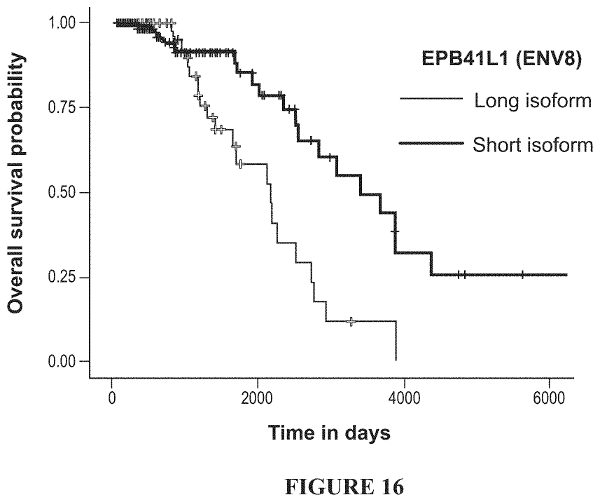

The criticality of alternative splicing events for TNMC tumors are also shown in FIG. 11, FIG. 12, FIG. 13 and FIG. 14. The alternative splicing events may be associated with one or more genes as described above or elsewhere herein, e.g., genes comprising NEDD4L (ENV2), MAP3K7 (ENV3), NFYA (ENV11), ESYT2 (ENV21), MARK2 (ENV18), ST7 (ENV19), ARVCF (ENV22), SYTL2 (ENV17), R3HDM1 (ENV23), COL4A3BP (ENV9), TANGO2 (ENV6), SEPT9 (ENV15), ROBO1 (ENV4), FAM122B (ENV5), CD47 (ENV13), LSR (ENV20), PBX1 (ENV16), EPB41 (ENV14), ADAM15 (ENV7), EPB41L1 (ENV8), ABI1 (ENV10), FLNB (ENV1), CTNND1 (ENV12), GPR160 (ENV24), ITGB3BP (ENV25), INCENP (ENV26), DENND1B (ENV27), CA12 (ENV28), or a combination thereof. Survival analysis of TCGA BRCA patients containing long and short isoforms for candidates is conducted with results illustrated in FIG. 15 and FIG. 16, respectively. The candidates may be one or more genes as described above or elsewhere herein, e.g., genes comprising NEDD4L (ENV2), MAP3K7 (ENV3), NFYA (ENV11), ESYT2 (ENV21), MARK2 (ENV18), ST7 (ENV19), ARVCF (ENV22), SYTL2 (ENV17), R3HDM1 (ENV23), COL4A3BP (ENV9), TANGO2 (ENV6), SEPT9 (ENV15), ROBO1 (ENV4), FAM122B (ENV5), CD47 (ENV13), LSR (ENV20), PBX1 (ENV 16), EPB41 (ENV14), ADAM (ENV7), EPB41L1 (ENV8), ABI1 (ENV10), FLNB (ENV1), CTNND1 (ENV12), GPR160 (ENV24), ITGB3BP (ENV25), INCENP (ENV26), DENND1B (ENV27), CA12 (ENV28), or a combination thereof. The analysis shows significant different in overall survival in patients expressing either of the isoforms.

Thus, the above results show that TNBC subtype can be vulnerable to splicing changes. Using the software platform of the present disclosure, reproducible and high impact splicing changes can be identified and validated in RNA-seq datasets from patient samples. The candidates listed can potentially serve as a therapeutic target for TNBC breast cancer. The platform has also designed and nominated oligonucleotide sequences that can promote splice switching when targeted using antisense oligos. The platform-designed oligos are experimentally validated for NEDD4L using uniformly modified 2′MOE oligonucleotide chemistry. The NEDD4L alternative splicing is a splicing event specific to TNBC subtype of breast cancer. Targeting NEDD4L isoform switching using modalities such as antisense oligonucleotides exhibits selective viability loss in TNBC cells specifically in a dose responsive manner. As such, NEDD4L splicing can be an actionable event to develop therapeutic to treat TNBC patients.

It shall be understood that although the above example is related to TNBC RNA-seq datasets, the NEDD4L and other candidate splicing events as discussed above or elsewhere herein can also be important in other solid or hematological malignancies, as well as in neurological or metabolic diseases. One or more of the splicing changes can be responsible for pathogenesis or progression of such diseases, disorders, or conditions, and the therapeutic targeting of the present disclosure can be applied to such diseases, disorders, or conditions.

Alternatively or additionally, the splicing targets can be modulated using modalities including, but not limited to, small molecules, GAPMER oligonucleotides, siRNAs, CAR-T cells, or any combination thereof to achieve disease-specific targeting. For example, some of the disease-specific splicing events that have been identified can open up grooves for small molecule binding. In such case, small molecules can be designed to target the region that is created because of a splicing change. In another example, splicing changes of the membrane bound proteins can display altered surface epitopes which can be specifically targeted using antibodies. The SpliceCore software platform, and the methods as described above and elsewhere herein, can be used to analyze, design, and develop multimodal therapeutic targets for a wide range of disease indications.

### Example 2

### Target-Specific Anti-Sense Oligonucleotides (ASOs)

As provided above and elsewhere herein, the ASOs can be identified based on exon position and SpliceLearn^{TM} features such as RNA binding protein. The ASOs can be used for splice switching experiments to promote exon inclusion in the target candidates provided herein. In some cases, chemically-modified ASOs may be synthesized based on the ASOs identified. For example, one or more chemical modifications can be introduced in one or more ASOs. The chemical modification can comprise a phosphorothioate backbone modification and/or 2′-O-(2 Methoxyethyl) ribose modification (2′MOE) (modification of the ribose sugar). Sequences of the ASOs may be used for one or more ex vivo experiments on breast cancer cell lines to determine the effect of the ASOs on splice switching of the target gene candidates.

A select group of the ASOs may be used for further in vitro and in vivo experiments and preclinical studies. In some instances, the specific splice switching event can be identified to be strongly associated with triple negative breast cancer (TNBC). In some embodiments, the ASOs may have potent splice switching effects with less toxicity. In some cases, the ASOs can be used in preclinical studies and/or in the therapeutic development of targeting of cancer-specific genes in patients. For example, an ASO can be used in the therapeutic development in the targeting of TNBC breast cancer patients by inducing a splice switch that can potentially have an anti-tumor effect.

Tables 2-8 list example target-specific oligonucleotide sequences of variable sequence lengths which may be used to induce an isoform switch or modulate (e.g., inhibit or enhance) the biological activity of a specific isoform of one or several of the genes described above or elsewhere herein (e.g., genes comprising one or more of NEDD4L (ENV2), MAP3K7 (ENV3), NFYA (ENV11), ESYT2 (ENV21), MARK2 (ENV18), ST7 (ENV19), ARVCF (ENV22), SYTL2 (ENV17), R3HDM1 (ENV23), COL4A3BP (ENV9), TANGO2 (ENV6), SEPT9 (ENV15), ROBO1 (ENV4), FAM122B (ENV5), CD47 (ENV13), LSR (ENV20), PBX1 (ENV16), EPB41 (ENV14), ADAM15 (ENV7), EPB41L1 (ENV8), ABI1 (ENV10), FLNB (ENV1), CTNND1 (ENV12), GPR160 (ENV24), ITGB3BP (ENV25), INCENP (ENV26), DENND1B (ENV27), CA12 (ENV28).

In some cases, oligonucleotide sequences comprised in a table are specific for a single target. In some cases, oligonucleotide sequences comprised in a table are specific for more than one target. In some cases, oligonucleotide sequences comprised in more than one tables are specific for a single target. For example, oligonucleotide sequences comprised in Tables 2-8 may be specific for a single target. The target may be a gene selected from genes described above or elsewhere herein.

TABLE 2
16-mer target-specific ASOs

| | | | | | | SEQ |
|---|---|---|---|---|---|---|
| CHR | START | END | STRAND | kmer | SEQUENCE | ID NO: |
| chr3 | 58141828 | 58141843 | +/− | 16 | CCCAACTAATCTCCAT | 6 |
| chr3 | 58141829 | 58141844 | +/− | 16 | CCAACTAATCTCCATT | 7 |
| chr3 | 58141830 | 58141845 | +/− | 16 | CAACTAATCTCCATTT | 8 |
| chr3 | 58141831 | 58141846 | +/− | 16 | AACTAATCTCCATTTG | 9 |
| chr3 | 58141832 | 58141847 | +/− | 16 | ACTAATCTCCATTTGC | 10 |
| chr3 | 58141833 | 58141848 | +/− | 16 | CTAATCTCCATTTGCC | 11 |
| chr3 | 58141834 | 58141849 | +/− | 16 | TAATCTCCATTTGCCA | 12 |
| chr3 | 58141835 | 58141850 | +/− | 16 | AATCTCCATTTGCCAC | 13 |
| chr3 | 58141836 | 58141851 | +/− | 16 | ATCTCCATTTGCCACT | 14 |
| chr3 | 58141837 | 58141852 | +/− | 16 | TCTCCATTTGCCACTG | 15 |
| chr3 | 58141838 | 58141853 | +/− | 16 | CTCCATTTGCCACTGA | 16 |
| chr3 | 58141839 | 58141854 | +/− | 16 | TCCATTTGCCACTGAC | 17 |
| chr3 | 58141840 | 58141855 | +/− | 16 | CCATTTGCCACTGACC | 18 |
| chr3 | 58141841 | 58141856 | +/− | 16 | CATTTGCCACTGACCA | 19 |
| chr3 | 58141842 | 58141857 | +/− | 16 | ATTTGCCACTGACCAG | 20 |
| chr3 | 58141843 | 58141858 | +/− | 16 | TTTGCCACTGACCAGG | 21 |
| chr3 | 58141844 | 58141859 | +/− | 16 | TTGCCACTGACCAGGC | 22 |
| chr3 | 58141845 | 58141860 | +/− | 16 | TGCCACTGACCAGGCC | 23 |
| chr3 | 58141846 | 58141861 | +/− | 16 | GCCACTGACCAGGCCA | 24 |
| chr3 | 58141847 | 58141862 | +/− | 16 | CCACTGACCAGGCCAC | 25 |
| chr3 | 58141848 | 58141863 | +/− | 16 | CACTGACCAGGCCACA | 26 |
| chr3 | 58141849 | 58141864 | +/− | 16 | ACTGACCAGGCCACAG | 27 |
| chr3 | 58141850 | 58141865 | +/− | 16 | CTGACCAGGCCACAGA | 28 |
| chr3 | 58141851 | 58141866 | +/− | 16 | TGACCAGGCCACAGAT | 29 |
| chr3 | 58141852 | 58141867 | +/− | 16 | GACCAGGCCACAGATG | 30 |
| chr3 | 58141853 | 58141868 | +/− | 16 | ACCAGGCCACAGATGG | 31 |
| chr3 | 58141854 | 58141869 | +/− | 16 | CCAGGCCACAGATGGG | 32 |
| chr3 | 58141855 | 58141870 | +/− | 16 | CAGGCCACAGATGGGG | 33 |
| chr3 | 58141856 | 58141871 | +/− | 16 | AGGCCACAGATGGGGA | 34 |
| chr3 | 58141857 | 58141872 | +/− | 16 | GGCCACAGATGGGGAA | 35 |
| chr3 | 58141858 | 58141873 | +/− | 16 | GCCACAGATGGGGAAG | 36 |
| chr3 | 58141859 | 58141874 | +/− | 16 | CCACAGATGGGGAAGT | 37 |
| chr3 | 58141860 | 58141875 | +/− | 16 | CACAGATGGGGAAGTC | 38 |
| chr3 | 58141861 | 58141876 | +/− | 16 | ACAGATGGGGAAGTCA | 39 |
| chr3 | 58141862 | 58141877 | +/− | 16 | CAGATGGGGAAGTCAC | 40 |
| chr3 | 58141863 | 58141878 | +/− | 16 | AGATGGGGAAGTCACA | 41 |
| chr3 | 58141864 | 58141879 | +/− | 16 | GATGGGGAAGTCACAG | 42 |
| chr3 | 58141865 | 58141880 | +/− | 16 | ATGGGGAAGTCACAGC | 43 |
| chr3 | 58141866 | 58141881 | +/− | 16 | TGGGGAAGTCACAGCC | 44 |
| chr3 | 58141867 | 58141882 | +/− | 16 | GGGGAAGTCACAGCCG | 45 |
| chr3 | 58141868 | 58141883 | +/− | 16 | GGGAAGTCACAGCCGT | 46 |
| chr3 | 58141869 | 58141884 | +/− | 16 | GGAAGTCACAGCCGTG | 47 |
| chr3 | 58141870 | 58141885 | +/− | 16 | GAAGTCACAGCCGTGG | 48 |
| chr3 | 58141871 | 58141886 | +/− | 16 | AAGTCACAGCCGTGGA | 49 |
| chr3 | 58141872 | 58141887 | +/− | 16 | AGTCACAGCCGTGGAG | 50 |
| chr3 | 58141873 | 58141888 | +/− | 16 | GTCACAGCCGTGGAGG | 51 |
| chr3 | 58141874 | 58141889 | +/− | 16 | TCACAGCCGTGGAGGA | 52 |
| chr3 | 58141875 | 58141890 | +/− | 16 | CACAGCCGTGGAGGAG | 53 |
| chr3 | 58141876 | 58141891 | +/− | 16 | ACAGCCGTGGAGGAGG | 54 |
| chr3 | 58141877 | 58141892 | +/− | 16 | CAGCCGTGGAGGAGGC | 55 |
| chr3 | 58141878 | 58141893 | +/− | 16 | AGCCGTGGAGGAGGCA | 56 |
| chr3 | 58141879 | 58141894 | +/− | 16 | GCCGTGGAGGAGGCAC | 57 |
| chr3 | 58141880 | 58141895 | +/− | 16 | CCGTGGAGGAGGCACC | 58 |
| chr3 | 58141881 | 58141896 | +/− | 16 | CGTGGAGGAGGCACCG | 59 |
| chr3 | 58141882 | 58141897 | +/− | 16 | GTGGAGGAGGCACCGG | 60 |
| chr3 | 58141883 | 58141898 | +/− | 16 | TGGAGGAGGCACCGGT | 61 |
| chr3 | 58141884 | 58141899 | +/− | 16 | GGAGGAGGCACCGGTA | 62 |
| chr3 | 58141885 | 58141900 | +/− | 16 | GAGGAGGCACCGGTAA | 63 |
| chr3 | 58141886 | 58141901 | +/− | 16 | AGGAGGCACCGGTAAA | 64 |
| chr3 | 58141887 | 58141902 | +/− | 16 | GGAGGCACCGGTAAAT | 65 |
| chr3 | 58141888 | 58141903 | +/− | 16 | GAGGCACCGGTAAATG | 66 |
| chr3 | 58141889 | 58141904 | +/− | 16 | AGGCACCGGTAAATGC | 67 |
| chr3 | 58141890 | 58141905 | +/− | 16 | GGCACCGGTAAATGCA | 68 |
| chr3 | 58141891 | 58141906 | +/− | 16 | GCACCGGTAAATGCAT | 69 |
| chr3 | 58141892 | 58141907 | +/− | 16 | CACCGGTAAATGCATG | 70 |
| chr3 | 58141893 | 58141908 | +/− | 16 | ACCGGTAAATGCATGT | 71 |
| chr3 | 58141894 | 58141909 | +/− | 16 | CCGGTAAATGCATGTC | 72 |
| chr3 | 58141895 | 58141910 | +/− | 16 | CGGTAAATGCATGTCC | 73 |
| chr3 | 58141896 | 58141911 | +/− | 16 | GGTAAATGCATGTCCC | 74 |
| chr3 | 58141897 | 58141912 | +/− | 16 | GTAAATGCATGTCCCC | 75 |
| chr3 | 58141898 | 58141913 | +/− | 16 | TAAATGCATGTCCCCC | 76 |
| chr3 | 58141899 | 58141914 | +/− | 16 | AAATGCATGTCCCCCT | 77 |
| chr3 | 58141900 | 58141915 | +/− | 16 | AATGCATGTCCCCCTG | 78 |
| chr3 | 58141901 | 58141916 | +/− | 16 | ATGCATGTCCCCCTGG | 79 |
| chr3 | 58141902 | 58141917 | +/− | 16 | TGCATGTCCCCCTGGA | 80 |
| chr3 | 58141903 | 58141918 | +/− | 16 | GCATGTCCCCCTGGAT | 81 |
| chr3 | 58141904 | 58141919 | +/− | 16 | CATGTCCCCCTGGATT | 82 |
| chr3 | 58141905 | 58141920 | +/− | 16 | ATGTCCCCCTGGATTC | 83 |
| chr3 | 58141906 | 58141921 | +/− | 16 | TGTCCCCCTGGATTCA | 84 |
| chr3 | 58141907 | 58141922 | +/− | 16 | GTCCCCCTGGATTCAG | 85 |
| chr3 | 58141908 | 58141923 | +/− | 16 | TCCCCCTGGATTCAGG | 86 |
| chr3 | 58141909 | 58141924 | +/− | 16 | CCCCCTGGATTCAGGC | 87 |
| chr3 | 58141910 | 58141925 | +/− | 16 | CCCCTGGATTCAGGCC | 88 |
| chr3 | 58141911 | 58141926 | +/− | 16 | CCCTGGATTCAGGCCC | 89 |
| chr3 | 58141912 | 58141927 | +/− | 16 | CCTGGATTCAGGCCCT | 90 |
| chr3 | 58141913 | 58141928 | +/− | 16 | CTGGATTCAGGCCCTG | 91 |
| chr3 | 58141914 | 58141929 | +/− | 16 | TGGATTCAGGCCCTGG | 92 |
| chr3 | 58141915 | 58141930 | +/− | 16 | GGATTCAGGCCCTGGG | 93 |
| chr3 | 58141916 | 58141931 | +/− | 16 | GATTCAGGCCCTGGGT | 94 |
| chr3 | 58141917 | 58141932 | +/− | 16 | ATTCAGGCCCTGGGTA | 95 |
| chr3 | 58141918 | 58141933 | +/− | 16 | TTCAGGCCCTGGGTAC | 96 |
| chr3 | 58141919 | 58141934 | +/− | 16 | TCAGGCCCTGGGTACA | 97 |
| chr3 | 58141920 | 58141935 | +/− | 16 | CAGGCCCTGGGTACAA | 98 |
| chr3 | 58141921 | 58141936 | +/− | 16 | AGGCCCTGGGTACAAT | 99 |
| chr3 | 58141922 | 58141937 | +/− | 16 | GGCCCTGGGTACAATT | 100 |
| chr3 | 58141923 | 58141938 | +/− | 16 | GCCCTGGGTACAATTT | 101 |
| chr3 | 58141924 | 58141939 | +/− | 16 | CCCTGGGTACAATTTT | 102 |
| chr3 | 58141925 | 58141940 | +/− | 16 | CCTGGGTACAATTTTG | 103 |
| chr3 | 58141926 | 58141941 | +/− | 16 | CTGGGTACAATTTTGG | 104 |
| chr3 | 58141927 | 58141942 | +/− | 16 | TGGGTACAATTTTGGT | 105 |
| chr3 | 58141928 | 58141943 | +/− | 16 | GGGTACAATTTTGGTT | 106 |
| chr3 | 58141929 | 58141944 | +/− | 16 | GGTACAATTTTGGTTT | 107 |
| chr3 | 58141930 | 58141945 | +/− | 16 | GTACAATTTTGGTTTT | 108 |
| chr3 | 58141931 | 58141946 | +/− | 16 | TACAATTTTGGTTTTT | 109 |
| chr3 | 58141932 | 58141947 | +/− | 16 | ACAATTTTGGTTTTTT | 110 |
| chr3 | 58141933 | 58141948 | +/− | 16 | CAATTTTGGTTTTTTC | 111 |
| chr3 | 58141934 | 58141949 | +/− | 16 | AATTTTGGTTTTTTCC | 112 |
| chr3 | 58141935 | 58141950 | +/− | 16 | ATTTTGGTTTTTTCCT | 113 |
| chr3 | 58141936 | 58141951 | +/− | 16 | TTTTGGTTTTTTCCTT | 114 |
| chr3 | 58141937 | 58141952 | +/− | 16 | TTTGGTTTTTTCCTTT | 115 |
| chr3 | 58141938 | 58141953 | +/− | 16 | TTGGTTTTTTCCTTTT | 116 |
| chr3 | 58141939 | 58141954 | +/− | 16 | TGGTTTTTTCCTTTTT | 117 |
| chr3 | 58141940 | 58141955 | +/− | 16 | GGTTTTTTCCTTTTTG | 118 |
| chr3 | 58141941 | 58141956 | +/− | 16 | GTTTTTTCCTTTTTGT | 119 |
| chr3 | 58141942 | 58141957 | +/− | 16 | TTTTTTCCTTTTTGTG | 120 |
| chr3 | 58141943 | 58141958 | +/− | 16 | TTTTTCCTTTTTGTGT | 121 |
| chr3 | 58141944 | 58141959 | +/− | 16 | TTTTCCTTTTTGTGTT | 122 |
| chr3 | 58141945 | 58141960 | +/− | 16 | TTTCCTTTTTGTGTTT | 123 |
| chr3 | 58141946 | 58141961 | +/− | 16 | TTCCTTTTTGTGTTTC | 124 |
| chr3 | 58141947 | 58141962 | +/− | 16 | TCCTTTTTGTGTTTCT | 125 |
| chr3 | 58141948 | 58141963 | +/− | 16 | CCTTTTTGTGTTTCTG | 126 |
| chr3 | 58141949 | 58141964 | +/− | 16 | CTTTTTGTGTTTCTGT | 127 |
| chr3 | 58141950 | 58141965 | +/− | 16 | TTTTTGTGTTTCTGTG | 128 |
| chr3 | 58141951 | 58141966 | +/− | 16 | TTTTGTGTTTCTGTGT | 129 |
| chr3 | 58141952 | 58141967 | +/− | 16 | TTTGTGTTTCTGTGTT | 130 |
| chr3 | 58141953 | 58141968 | +/− | 16 | TTGTGTTTCTGTGTTT | 131 |
| chr3 | 58141954 | 58141969 | +/− | 16 | TGTGTTTCTGTGTTTA | 132 |
| chr3 | 58141955 | 58141970 | +/− | 16 | GTGTTTCTGTGTTTAC | 133 |
| chr3 | 58141956 | 58141971 | +/− | 16 | TGTTTCTGTGTTTACT | 134 |
| chr3 | 58141957 | 58141972 | +/− | 16 | GTTTCTGTGTTTACTC | 135 |
| chr3 | 58141958 | 58141973 | +/− | 16 | TTTCTGTGTTTACTCA | 136 |
| chr3 | 58141959 | 58141974 | +/− | 16 | TTCTGTGTTTACTCAG | 137 |
| chr3 | 58141960 | 58141975 | +/− | 16 | TCTGTGTTTACTCAGC | 138 |
| chr3 | 58141961 | 58141976 | +/− | 16 | CTGTGTTTACTCAGCC | 139 |
| chr3 | 58141962 | 58141977 | +/− | 16 | TGTGTTTACTCAGCCT | 140 |
| chr3 | 58141963 | 58141978 | +/− | 16 | GTGTTTACTCAGCCTT | 141 |
| chr3 | 58141964 | 58141979 | +/− | 16 | TGTTTACTCAGCCTTC | 142 |
| chr3 | 58141965 | 58141980 | +/− | 16 | GTTTACTCAGCCTTCA | 143 |
| chr3 | 58141966 | 58141981 | +/− | 16 | TTTACTCAGCCTTCAT | 144 |
| chr3 | 58141967 | 58141982 | +/− | 16 | TTACTCAGCCTTCATT | 145 |
| chr3 | 58141968 | 58141983 | +/− | 16 | TACTCAGCCTTCATTT | 146 |
| chr3 | 58141969 | 58141984 | +/− | 16 | ACTCAGCCTTCATTTC | 147 |
| chr3 | 58141970 | 58141985 | +/− | 16 | CTCAGCCTTCATTTCA | 148 |
| chr3 | 58141971 | 58141986 | +/− | 16 | TCAGCCTTCATTTCAG | 149 |
| chr3 | 58141972 | 58141987 | +/− | 16 | CAGCCTTCATTTCAGA | 150 |
| chr3 | 58141973 | 58141988 | +/− | 16 | AGCCTTCATTTCAGAA | 151 |
| chr3 | 58141974 | 58141989 | +/− | 16 | GCCTTCATTTCAGAAA | 152 |
| chr3 | 58141975 | 58141990 | +/− | 16 | CCTTCATTTCAGAAAA | 153 |
| chr3 | 58141976 | 58141991 | +/− | 16 | CTTCATTTCAGAAAAT | 154 |
| chr3 | 58141977 | 58141992 | +/− | 16 | TTCATTTCAGAAAATC | 155 |
| chr3 | 58141978 | 58141993 | +/− | 16 | TCATTTCAGAAAATCT | 156 |
| chr3 | 58141979 | 58141994 | +/− | 16 | CATTTCAGAAAATCTG | 157 |
| chr3 | 58141980 | 58141995 | +/− | 16 | ATTTCAGAAAATCTGC | 158 |
| chr3 | 58141981 | 58141996 | +/− | 16 | TTTCAGAAAATCTGCC | 159 |
| chr3 | 58141982 | 58141997 | +/− | 16 | TTCAGAAAATCTGCCA | 160 |
| chr3 | 58141983 | 58141998 | +/− | 16 | TCAGAAAATCTGCCAT | 161 |
| chr3 | 58141984 | 58141999 | +/− | 16 | CAGAAAATCTGCCATC | 162 |
| chr3 | 58141985 | 58142000 | +/− | 16 | AGAAAATCTGCCATCT | 163 |
| chr3 | 58141986 | 58142001 | +/− | 16 | GAAAATCTGCCATCTG | 164 |
| chr3 | 58141987 | 58142002 | +/− | 16 | AAAATCTGCCATCTGC | 165 |
| chr3 | 58141988 | 58142003 | +/− | 16 | AAATCTGCCATCTGCT | 166 |
| chr3 | 58141989 | 58142004 | +/− | 16 | AATCTGCCATCTGCTT | 167 |
| chr3 | 58141990 | 58142005 | +/− | 16 | ATCTGCCATCTGCTTC | 168 |
| chr3 | 58141991 | 58142006 | +/− | 16 | TCTGCCATCTGCTTCT | 169 |
| chr3 | 58141992 | 58142007 | +/− | 16 | CTGCCATCTGCTTCTG | 170 |
| chr3 | 58141993 | 58142008 | +/− | 16 | TGCCATCTGCTTCTGG | 171 |
| chr3 | 58141994 | 58142009 | +/− | 16 | GCCATCTGCTTCTGGG | 172 |
| chr3 | 58141995 | 58142010 | +/− | 16 | CCATCTGCTTCTGGGA | 173 |
| chr3 | 58141996 | 58142011 | +/− | 16 | CATCTGCTTCTGGGAT | 174 |
| chr3 | 58141997 | 58142012 | +/− | 16 | ATCTGCTTCTGGGATT | 175 |
| chr3 | 58141998 | 58142013 | +/− | 16 | TCTGCTTCTGGGATTG | 176 |
| chr3 | 58141999 | 58142014 | +/− | 16 | CTGCTTCTGGGATTGC | 177 |
| chr3 | 58142000 | 58142015 | +/− | 16 | TGCTTCTGGGATTGCT | 178 |
| chr3 | 58142001 | 58142016 | +/− | 16 | GCTTCTGGGATTGCTT | 179 |
| chr3 | 58142002 | 58142017 | +/− | 16 | CTTCTGGGATTGCTTA | 180 |
| chr3 | 58142003 | 58142018 | +/− | 16 | TTCTGGGATTGCTTAA | 181 |
| chr3 | 58142004 | 58142019 | +/− | 16 | TCTGGGATTGCTTAAG | 182 |
| chr3 | 58142005 | 58142020 | +/− | 16 | CTGGGATTGCTTAAGC | 183 |
| chr3 | 58142006 | 58142021 | +/− | 16 | TGGGATTGCTTAAGCC | 184 |
| chr3 | 58142007 | 58142022 | +/− | 16 | GGGATTGCTTAAGCCC | 185 |
| chr3 | 58142008 | 58142023 | +/− | 16 | GGATTGCTTAAGCCCT | 186 |
| chr3 | 58142009 | 58142024 | +/− | 16 | GATTGCTTAAGCCCTG | 187 |
| chr3 | 58142010 | 58142025 | +/− | 16 | ATTGCTTAAGCCCTGT | 188 |
| chr3 | 58142011 | 58142026 | +/− | 16 | TTGCTTAAGCCCTGTG | 189 |
| chr3 | 58142012 | 58142027 | +/− | 16 | TGCTTAAGCCCTGTGG | 190 |
| chr3 | 58142013 | 58142028 | +/− | 16 | GCTTAAGCCCTGTGGG | 191 |
| chr3 | 58142014 | 58142029 | +/− | 16 | CTTAAGCCCTGTGGGT | 192 |
| chr3 | 58142015 | 58142030 | +/− | 16 | TTAAGCCCTGTGGGTG | 193 |
| chr3 | 58142016 | 58142031 | +/− | 16 | TAAGCCCTGTGGGTGT | 194 |
| chr3 | 58142017 | 58142032 | +/− | 16 | AAGCCCTGTGGGTGTC | 195 |
| chr3 | 58142018 | 58142033 | +/− | 16 | AGCCCTGTGGGTGTCC | 196 |
| chr3 | 58142019 | 58142034 | +/− | 16 | GCCCTGTGGGTGTCCT | 197 |
| chr3 | 58142020 | 58142035 | +/− | 16 | CCCTGTGGGTGTCCTG | 198 |
| chr3 | 58142021 | 58142036 | +/− | 16 | CCTGTGGGTGTCCTGG | 199 |
| chr3 | 58142022 | 58142037 | +/− | 16 | CTGTGGGTGTCCTGGT | 200 |
| chr3 | 58142023 | 58142038 | +/− | 16 | TGTGGGTGTCCTGGTC | 201 |
| chr3 | 58142024 | 58142039 | +/− | 16 | GTGGGTGTCCTGGTCA | 202 |
| chr3 | 58142025 | 58142040 | +/− | 16 | TGGGTGTCCTGGTCAT | 203 |
| chr3 | 58142026 | 58142041 | +/− | 16 | GGGTGTCCTGGTCATT | 204 |
| chr3 | 58142027 | 58142042 | +/− | 16 | GGTGTCCTGGTCATTG | 205 |
| chr3 | 58142028 | 58142043 | +/− | 16 | GTGTCCTGGTCATTGG | 206 |
| chr3 | 58142029 | 58142044 | +/− | 16 | TGTCCTGGTCATTGGT | 207 |

TABLE 3
17-mer target-specific ASOs

| | | | | | | SEQ |
|---|---|---|---|---|---|---|
| | | | | | | ID |
| CHR | START | END | STRAND | kmer | SEQUENCE | NO: |
| chr3 | 58141828 | 58141844 | +/− | 17 | CCCAACTAATCTCCATT | 208 |
| chr3 | 58141829 | 58141845 | +/− | 17 | CCAACTAATCTCCATTT | 209 |
| chr3 | 58141830 | 58141846 | +/− | 17 | CAACTAATCTCCATTTG | 210 |
| chr3 | 58141831 | 58141847 | +/− | 17 | AACTAATCTCCATTTGC | 211 |
| chr3 | 58141832 | 58141848 | +/− | 17 | ACTAATCTCCATTTGCC | 212 |
| chr3 | 58141833 | 58141849 | +/− | 17 | CTAATCTCCATTTGCCA | 213 |
| chr3 | 58141834 | 58141850 | +/− | 17 | TAATCTCCATTTGCCAC | 214 |
| chr3 | 58141835 | 58141851 | +/− | 17 | AATCTCCATTTGCCACT | 215 |
| chr3 | 58141836 | 58141852 | +/− | 17 | ATCTCCATTTGCCACTG | 216 |
| chr3 | 58141837 | 58141853 | +/− | 17 | TCTCCATTTGCCACTGA | 217 |
| chr3 | 58141838 | 58141854 | +/− | 17 | CTCCATTTGCCACTGAC | 218 |
| chr3 | 58141839 | 58141855 | +/− | 17 | TCCATTTGCCACTGACC | 219 |
| chr3 | 58141840 | 58141856 | +/− | 17 | CCATTTGCCACTGACCA | 220 |
| chr3 | 58141841 | 58141857 | +/− | 17 | CATTTGCCACTGACCAG | 221 |
| chr3 | 58141842 | 58141858 | +/− | 17 | ATTTGCCACTGACCAGG | 222 |
| chr3 | 58141843 | 58141859 | +/− | 17 | TTTGCCACTGACCAGGC | 223 |
| chr3 | 58141844 | 58141860 | +/− | 17 | TTGCCACTGACCAGGCC | 224 |
| chr3 | 58141845 | 58141861 | +/− | 17 | TGCCACTGACCAGGCCA | 225 |
| chr3 | 58141846 | 58141862 | +/− | 17 | GCCACTGACCAGGCCAC | 226 |
| chr3 | 58141847 | 58141863 | +/− | 17 | CCACTGACCAGGCCACA | 227 |
| chr3 | 58141848 | 58141864 | +/− | 17 | CACTGACCAGGCCACAG | 228 |
| chr3 | 58141849 | 58141865 | +/− | 17 | ACTGACCAGGCCACAGA | 229 |
| chr3 | 58141850 | 58141866 | +/− | 17 | CTGACCAGGCCACAGAT | 230 |
| chr3 | 58141851 | 58141867 | +/− | 17 | TGACCAGGCCACAGATG | 231 |
| chr3 | 58141852 | 58141868 | +/− | 17 | GACCAGGCCACAGATGG | 232 |
| chr3 | 58141853 | 58141869 | +/− | 17 | ACCAGGCCACAGATGGG | 233 |
| chr3 | 58141854 | 58141870 | +/− | 17 | CCAGGCCACAGATGGGG | 234 |
| chr3 | 58141855 | 58141871 | +/− | 17 | CAGGCCACAGATGGGGA | 235 |
| chr3 | 58141856 | 58141872 | +/− | 17 | AGGCCACAGATGGGGAA | 236 |
| chr3 | 58141857 | 58141873 | +/− | 17 | GGCCACAGATGGGGAAG | 237 |
| chr3 | 58141858 | 58141874 | +/− | 17 | GCCACAGATGGGGAAGT | 238 |
| chr3 | 58141859 | 58141875 | +/− | 17 | CCACAGATGGGGAAGTC | 239 |
| chr3 | 58141860 | 58141876 | +/− | 17 | CACAGATGGGGAAGTCA | 240 |
| chr3 | 58141861 | 58141877 | +/− | 17 | ACAGATGGGGAAGTCAC | 241 |
| chr3 | 58141862 | 58141878 | +/− | 17 | CAGATGGGGAAGTCACA | 242 |
| chr3 | 58141863 | 58141879 | +/− | 17 | AGATGGGGAAGTCACAG | 243 |
| chr3 | 58141864 | 58141880 | +/− | 17 | GATGGGGAAGTCACAGC | 244 |
| chr3 | 58141865 | 58141881 | +/− | 17 | ATGGGGAAGTCACAGCC | 245 |
| chr3 | 58141866 | 58141882 | +/− | 17 | TGGGGAAGTCACAGCCG | 246 |
| chr3 | 58141867 | 58141883 | +/− | 17 | GGGGAAGTCACAGCCGT | 247 |
| chr3 | 58141868 | 58141884 | +/− | 17 | GGGAAGTCACAGCCGTG | 248 |
| chr3 | 58141869 | 58141885 | +/− | 17 | GGAAGTCACAGCCGTGG | 249 |
| chr3 | 58141870 | 58141886 | +/− | 17 | GAAGTCACAGCCGTGGA | 250 |
| chr3 | 58141871 | 58141887 | +/− | 17 | AAGTCACAGCCGTGGAG | 251 |
| chr3 | 58141872 | 58141888 | +/− | 17 | AGTCACAGCCGTGGAGG | 252 |
| chr3 | 58141873 | 58141889 | +/− | 17 | GTCACAGCCGTGGAGGA | 253 |
| chr3 | 58141874 | 58141890 | +/− | 17 | TCACAGCCGTGGAGGAG | 254 |
| chr3 | 58141875 | 58141891 | +/− | 17 | CACAGCCGTGGAGGAGG | 255 |
| chr3 | 58141876 | 58141892 | +/− | 17 | ACAGCCGTGGAGGAGGC | 256 |
| chr3 | 58141877 | 58141893 | +/− | 17 | CAGCCGTGGAGGAGGCA | 257 |
| chr3 | 58141878 | 58141894 | +/− | 17 | AGCCGTGGAGGAGGCAC | 258 |
| chr3 | 58141879 | 58141895 | +/− | 17 | GCCGTGGAGGAGGCACC | 259 |
| chr3 | 58141880 | 58141896 | +/− | 17 | CCGTGGAGGAGGCACCG | 260 |
| chr3 | 58141881 | 58141897 | +/− | 17 | CGTGGAGGAGGCACCGG | 261 |
| chr3 | 58141882 | 58141898 | +/− | 17 | GTGGAGGAGGCACCGGT | 262 |
| chr3 | 58141883 | 58141899 | +/− | 17 | TGGAGGAGGCACCGGTA | 263 |
| chr3 | 58141884 | 58141900 | +/− | 17 | GGAGGAGGCACCGGTAA | 264 |
| chr3 | 58141885 | 58141901 | +/− | 17 | GAGGAGGCACCGGTAAA | 265 |
| chr3 | 58141886 | 58141902 | +/− | 17 | AGGAGGCACCGGTAAAT | 266 |
| chr3 | 58141887 | 58141903 | +/− | 17 | GGAGGCACCGGTAAATG | 267 |
| chr3 | 58141888 | 58141904 | +/− | 17 | GAGGCACCGGTAAATGC | 268 |
| chr3 | 58141889 | 58141905 | +/− | 17 | AGGCACCGGTAAATGCA | 269 |
| chr3 | 58141890 | 58141906 | +/− | 17 | GGCACCGGTAAATGCAT | 270 |
| chr3 | 58141891 | 58141907 | +/− | 17 | GCACCGGTAAATGCATG | 271 |
| chr3 | 58141892 | 58141908 | +/− | 17 | CACCGGTAAATGCATGT | 272 |
| chr3 | 58141893 | 58141909 | +/− | 17 | ACCGGTAAATGCATGTC | 273 |
| chr3 | 58141894 | 58141910 | +/− | 17 | CCGGTAAATGCATGTCC | 274 |
| chr3 | 58141895 | 58141911 | +/− | 17 | CGGTAAATGCATGTCCC | 275 |
| chr3 | 58141896 | 58141912 | +/− | 17 | GGTAAATGCATGTCCCC | 276 |
| chr3 | 58141897 | 58141913 | +/− | 17 | GTAAATGCATGTCCCCC | 277 |
| chr3 | 58141898 | 58141914 | +/− | 17 | TAAATGCATGTCCCCCT | 278 |
| chr3 | 58141899 | 58141915 | +/− | 17 | AAATGCATGTCCCCCTG | 279 |
| chr3 | 58141900 | 58141916 | +/− | 17 | AATGCATGTCCCCCTGG | 280 |
| chr3 | 58141901 | 58141917 | +/− | 17 | ATGCATGTCCCCCTGGA | 281 |
| chr3 | 58141902 | 58141918 | +/− | 17 | TGCATGTCCCCCTGGAT | 282 |
| chr3 | 58141903 | 58141919 | +/− | 17 | GCATGTCCCCCTGGATT | 283 |
| chr3 | 58141904 | 58141920 | +/− | 17 | CATGTCCCCCTGGATTC | 284 |
| chr3 | 58141905 | 58141921 | +/− | 17 | ATGTCCCCCTGGATTCA | 285 |
| chr3 | 58141906 | 58141922 | +/− | 17 | TGTCCCCCTGGATTCAG | 286 |
| chr3 | 58141907 | 58141923 | +/− | 17 | GTCCCCCTGGATTCAGG | 287 |
| chr3 | 58141908 | 58141924 | +/− | 17 | TCCCCCTGGATTCAGGC | 288 |
| chr3 | 58141909 | 58141925 | +/− | 17 | CCCCCTGGATTCAGGCC | 289 |
| chr3 | 58141910 | 58141926 | +/− | 17 | CCCCTGGATTCAGGCCC | 290 |
| chr3 | 58141911 | 58141927 | +/− | 17 | CCCTGGATTCAGGCCCT | 291 |
| chr3 | 58141912 | 58141928 | +/− | 17 | CCTGGATTCAGGCCCTG | 292 |
| chr3 | 58141913 | 58141929 | +/− | 17 | CTGGATTCAGGCCCTGG | 293 |
| chr3 | 58141914 | 58141930 | +/− | 17 | TGGATTCAGGCCCTGGG | 294 |
| chr3 | 58141915 | 58141931 | +/− | 17 | GGATTCAGGCCCTGGGT | 295 |
| chr3 | 58141916 | 58141932 | +/− | 17 | GATTCAGGCCCTGGGTA | 296 |
| chr3 | 58141917 | 58141933 | +/− | 17 | ATTCAGGCCCTGGGTAC | 297 |
| chr3 | 58141918 | 58141934 | +/− | 17 | TTCAGGCCCTGGGTACA | 298 |
| chr3 | 58141919 | 58141935 | +/− | 17 | TCAGGCCCTGGGTACAA | 299 |
| chr3 | 58141920 | 58141936 | +/− | 17 | CAGGCCCTGGGTACAAT | 300 |
| chr3 | 58141921 | 58141937 | +/− | 17 | AGGCCCTGGGTACAATT | 301 |
| chr3 | 58141922 | 58141938 | +/− | 17 | GGCCCTGGGTACAATTT | 302 |
| chr3 | 58141923 | 58141939 | +/− | 17 | GCCCTGGGTACAATTTT | 303 |
| chr3 | 58141924 | 58141940 | +/− | 17 | CCCTGGGTACAATTTTG | 304 |
| chr3 | 58141925 | 58141941 | +/− | 17 | CCTGGGTACAATTTTGG | 305 |
| chr3 | 58141926 | 58141942 | +/− | 17 | CTGGGTACAATTTTGGT | 306 |
| chr3 | 58141927 | 58141943 | +/− | 17 | TGGGTACAATTTTGGTT | 307 |
| chr3 | 58141928 | 58141944 | +/− | 17 | GGGTACAATTTTGGTTT | 308 |
| chr3 | 58141929 | 58141945 | +/− | 17 | GGTACAATTTTGGTTTT | 309 |
| chr3 | 58141930 | 58141946 | +/− | 17 | GTACAATTTTGGTTTTT | 310 |
| chr3 | 58141931 | 58141947 | +/− | 17 | TACAATTTTGGTTTTTT | 311 |
| chr3 | 58141932 | 58141948 | +/− | 17 | ACAATTTTGGTTTTTTC | 312 |
| chr3 | 58141933 | 58141949 | +/− | 17 | CAATTTTGGTTTTTTCC | 313 |
| chr3 | 58141934 | 58141950 | +/− | 17 | AATTTTGGTTTTTTCCT | 314 |
| chr3 | 58141935 | 58141951 | +/− | 17 | ATTTTGGTTTTTTCCTT | 315 |
| chr3 | 58141936 | 58141952 | +/− | 17 | TTTTGGTTTTTTCCTTT | 316 |
| chr3 | 58141937 | 58141953 | +/− | 17 | TTTGGTTTTTTCCTTTT | 317 |
| chr3 | 58141938 | 58141954 | +/− | 17 | TTGGTTTTTTCCTTTTT | 318 |
| chr3 | 58141939 | 58141955 | +/− | 17 | TGGTTTTTTCCTTTTTG | 319 |
| chr3 | 58141940 | 58141956 | +/− | 17 | GGTTTTTTCCTTTTTGT | 320 |
| chr3 | 58141941 | 58141957 | +/− | 17 | GTTTTTTCCTTTTTGTG | 321 |
| chr3 | 58141942 | 58141958 | +/− | 17 | TTTTTTCCTTTTTGTGT | 322 |
| chr3 | 58141943 | 58141959 | +/− | 17 | TTTTTCCTTTTTGTGTT | 323 |
| chr3 | 58141944 | 58141960 | +/− | 17 | TTTTCCTTTTTGTGTTT | 324 |
| chr3 | 58141945 | 58141961 | +/− | 17 | TTTCCTTTTTGTGTTTC | 325 |
| chr3 | 58141946 | 58141962 | +/− | 17 | TTCCTTTTTGTGTTTCT | 326 |
| chr3 | 58141947 | 58141963 | +/− | 17 | TCCTTTTTGTGTTTCTG | 327 |
| chr3 | 58141948 | 58141964 | +/− | 17 | CCTTTTTGTGTTTCTGT | 328 |
| chr3 | 58141949 | 58141965 | +/− | 17 | CTTTTTGTGTTTCTGTG | 329 |
| chr3 | 58141950 | 58141966 | +/− | 17 | TTTTTGTGTTTCTGTGT | 330 |
| chr3 | 58141951 | 58141967 | +/− | 17 | TTTTGTGTTTCTGTGTT | 331 |
| chr3 | 58141952 | 58141968 | +/− | 17 | TTTGTGTTTCTGTGTTT | 332 |
| chr3 | 58141953 | 58141969 | +/− | 17 | TTGTGTTTCTGTGTTTA | 333 |
| chr3 | 58141954 | 58141970 | +/− | 17 | TGTGTTTCTGTGTTTAC | 334 |
| chr3 | 58141955 | 58141971 | +/− | 17 | GTGTTTCTGTGTTTACT | 335 |
| chr3 | 58141956 | 58141972 | +/− | 17 | TGTTTCTGTGTTTACTC | 336 |
| chr3 | 58141957 | 58141973 | +/− | 17 | GTTTCTGTGTTTACTCA | 337 |
| chr3 | 58141958 | 58141974 | +/− | 17 | TTTCTGTGTTTACTCAG | 338 |
| chr3 | 58141959 | 58141975 | +/− | 17 | TTCTGTGTTTACTCAGC | 339 |
| chr3 | 58141960 | 58141976 | +/− | 17 | TCTGTGTTTACTCAGCC | 340 |
| chr3 | 58141961 | 58141977 | +/− | 17 | CTGTGTTTACTCAGCCT | 341 |
| chr3 | 58141962 | 58141978 | +/− | 17 | TGTGTTTACTCAGCCTT | 342 |
| chr3 | 58141963 | 58141979 | +/− | 17 | GTGTTTACTCAGCCTTC | 343 |
| chr3 | 58141964 | 58141980 | +/− | 17 | TGTTTACTCAGCCTTCA | 344 |
| chr3 | 58141965 | 58141981 | +/− | 17 | GTTTACTCAGCCTTCAT | 345 |
| chr3 | 58141966 | 58141982 | +/− | 17 | TTTACTCAGCCTTCATT | 346 |
| chr3 | 58141967 | 58141983 | +/− | 17 | TTACTCAGCCTTCATTT | 347 |
| chr3 | 58141968 | 58141984 | +/− | 17 | TACTCAGCCTTCATTTC | 348 |
| chr3 | 58141969 | 58141985 | +/− | 17 | ACTCAGCCTTCATTTCA | 349 |
| chr3 | 58141970 | 58141986 | +/− | 17 | CTCAGCCTTCATTTCAG | 350 |
| chr3 | 58141971 | 58141987 | +/− | 17 | TCAGCCTTCATTTCAGA | 351 |
| chr3 | 58141972 | 58141988 | +/− | 17 | CAGCCTTCATTTCAGAA | 352 |
| chr3 | 58141973 | 58141989 | +/− | 17 | AGCCTTCATTTCAGAAA | 353 |
| chr3 | 58141974 | 58141990 | +/− | 17 | GCCTTCATTTCAGAAAA | 354 |
| chr3 | 58141975 | 58141991 | +/− | 17 | CCTTCATTTCAGAAAAT | 355 |
| chr3 | 58141976 | 58141992 | +/− | 17 | CTTCATTTCAGAAAATC | 356 |
| chr3 | 58141977 | 58141993 | +/− | 17 | TTCATTTCAGAAAATCT | 357 |
| chr3 | 58141978 | 58141994 | +/− | 17 | TCATTTCAGAAAATCTG | 358 |
| chr3 | 58141979 | 58141995 | +/− | 17 | CATTTCAGAAAATCTGC | 359 |
| chr3 | 58141980 | 58141996 | +/− | 17 | ATTTCAGAAAATCTGCC | 360 |
| chr3 | 58141981 | 58141997 | +/− | 17 | TTTCAGAAAATCTGCCA | 361 |
| chr3 | 58141982 | 58141998 | +/− | 17 | TTCAGAAAATCTGCCAT | 362 |
| chr3 | 58141983 | 58141999 | +/− | 17 | TCAGAAAATCTGCCATC | 363 |
| chr3 | 58141984 | 58142000 | +/− | 17 | CAGAAAATCTGCCATCT | 364 |
| chr3 | 58141985 | 58142001 | +/− | 17 | AGAAAATCTGCCATCTG | 365 |
| chr3 | 58141986 | 58142002 | +/− | 17 | GAAAATCTGCCATCTGC | 366 |
| chr3 | 58141987 | 58142003 | +/− | 17 | AAAATCTGCCATCTGCT | 367 |
| chr3 | 58141988 | 58142004 | +/− | 17 | AAATCTGCCATCTGCTT | 368 |
| chr3 | 58141989 | 58142005 | +/− | 17 | AATCTGCCATCTGCTTC | 369 |
| chr3 | 58141990 | 58142006 | +/− | 17 | ATCTGCCATCTGCTTCT | 370 |
| chr3 | 58141991 | 58142007 | +/− | 17 | TCTGCCATCTGCTTCTG | 371 |
| chr3 | 58141992 | 58142008 | +/− | 17 | CTGCCATCTGCTTCTGG | 372 |
| chr3 | 58141993 | 58142009 | +/− | 17 | TGCCATCTGCTTCTGGG | 373 |
| chr3 | 58141994 | 58142010 | +/− | 17 | GCCATCTGCTTCTGGGA | 374 |
| chr3 | 58141995 | 58142011 | +/− | 17 | CCATCTGCTTCTGGGAT | 375 |
| chr3 | 58141996 | 58142012 | +/− | 17 | CATCTGCTTCTGGGATT | 376 |
| chr3 | 58141997 | 58142013 | +/− | 17 | ATCTGCTTCTGGGATTG | 377 |
| chr3 | 58141998 | 58142014 | +/− | 17 | TCTGCTTCTGGGATTGC | 378 |
| chr3 | 58141999 | 58142015 | +/− | 17 | CTGCTTCTGGGATTGCT | 379 |
| chr3 | 58142000 | 58142016 | +/− | 17 | TGCTTCTGGGATTGCTT | 380 |
| chr3 | 58142001 | 58142017 | +/− | 17 | GCTTCTGGGATTGCTTA | 381 |
| chr3 | 58142002 | 58142018 | +/− | 17 | CTTCTGGGATTGCTTAA | 382 |
| chr3 | 58142003 | 58142019 | +/− | 17 | TTCTGGGATTGCTTAAG | 383 |
| chr3 | 58142004 | 58142020 | +/− | 17 | TCTGGGATTGCTTAAGC | 384 |
| chr3 | 58142005 | 58142021 | +/− | 17 | CTGGGATTGCTTAAGCC | 385 |
| chr3 | 58142006 | 58142022 | +/− | 17 | TGGGATTGCTTAAGCCC | 386 |
| chr3 | 58142007 | 58142023 | +/− | 17 | GGGATTGCTTAAGCCCT | 387 |
| chr3 | 58142008 | 58142024 | +/− | 17 | GGATTGCTTAAGCCCTG | 388 |
| chr3 | 58142009 | 58142025 | +/− | 17 | GATTGCTTAAGCCCTGT | 389 |
| chr3 | 58142010 | 58142026 | +/− | 17 | ATTGCTTAAGCCCTGTG | 390 |
| chr3 | 58142011 | 58142027 | +/− | 17 | TTGCTTAAGCCCTGTGG | 391 |
| chr3 | 58142012 | 58142028 | +/− | 17 | TGCTTAAGCCCTGTGGG | 392 |
| chr3 | 58142013 | 58142029 | +/− | 17 | GCTTAAGCCCTGTGGGT | 393 |
| chr3 | 58142014 | 58142030 | +/− | 17 | CTTAAGCCCTGTGGGTG | 394 |
| chr3 | 58142015 | 58142031 | +/− | 17 | TTAAGCCCTGTGGGTGT | 395 |
| chr3 | 58142016 | 58142032 | +/− | 17 | TAAGCCCTGTGGGTGTC | 396 |
| chr3 | 58142017 | 58142033 | +/− | 17 | AAGCCCTGTGGGTGTCC | 397 |
| chr3 | 58142018 | 58142034 | +/− | 17 | AGCCCTGTGGGTGTCCT | 398 |
| chr3 | 58142019 | 58142035 | +/− | 17 | GCCCTGTGGGTGTCCTG | 399 |
| chr3 | 58142020 | 58142036 | +/− | 17 | CCCTGTGGGTGTCCTGG | 400 |
| chr3 | 58142021 | 58142037 | +/− | 17 | CCTGTGGGTGTCCTGGT | 401 |
| chr3 | 58142022 | 58142038 | +/− | 17 | CTGTGGGTGTCCTGGTC | 402 |
| chr3 | 58142023 | 58142039 | +/− | 17 | TGTGGGTGTCCTGGTCA | 403 |
| chr3 | 58142024 | 58142040 | +/− | 17 | GTGGGTGTCCTGGTCAT | 404 |
| chr3 | 58142025 | 58142041 | +/− | 17 | TGGGTGTCCTGGTCATT | 405 |
| chr3 | 58142026 | 58142042 | +/− | 17 | GGGTGTCCTGGTCATTG | 406 |
| chr3 | 58142027 | 58142043 | +/− | 17 | GGTGTCCTGGTCATTGG | 407 |
| chr3 | 58142028 | 58142044 | +/− | 17 | GTGTCCTGGTCATTGGT | 408 |

TABLE 4
18-mer target-specific ASOs

| | | | | | | SEQ |
|---|---|---|---|---|---|---|
| | | | | | | ID |
| CHR | START | END | STRAND | kmer | SEQUENCE | NO: |
| chr3 | 58141828 | 58141845 | +/− | 18 | CCCAACTAATCTCCATTT | 409 |
| chr3 | 58141829 | 58141846 | +/− | 18 | CCAACTAATCTCCATTTG | 410 |
| chr3 | 58141830 | 58141847 | +/− | 18 | CAACTAATCTCCATTTGC | 411 |
| chr3 | 58141831 | 58141848 | +/− | 18 | AACTAATCTCCATTTGCC | 412 |
| chr3 | 58141832 | 58141849 | +/− | 18 | ACTAATCTCCATTTGCCA | 413 |
| chr3 | 58141833 | 58141850 | +/− | 18 | CTAATCTCCATTTGCCAC | 414 |
| chr3 | 58141834 | 58141851 | +/− | 18 | TAATCTCCATTTGCCACT | 415 |
| chr3 | 58141835 | 58141852 | +/− | 18 | AATCTCCATTTGCCACTG | 416 |
| chr3 | 58141836 | 58141853 | +/− | 18 | ATCTCCATTTGCCACTGA | 417 |
| chr3 | 58141837 | 58141854 | +/− | 18 | TCTCCATTTGCCACTGAC | 418 |
| chr3 | 58141838 | 58141855 | +/− | 18 | CTCCATTTGCCACTGACC | 419 |
| chr3 | 58141839 | 58141856 | +/− | 18 | TCCATTTGCCACTGACCA | 420 |
| chr3 | 58141840 | 58141857 | +/− | 18 | CCATTTGCCACTGACCAG | 421 |
| chr3 | 58141841 | 58141858 | +/− | 18 | CATTTGCCACTGACCAGG | 422 |
| chr3 | 58141842 | 58141859 | +/− | 18 | ATTTGCCACTGACCAGGC | 423 |
| chr3 | 58141843 | 58141860 | +/− | 18 | TTTGCCACTGACCAGGCC | 424 |
| chr3 | 58141844 | 58141861 | +/− | 18 | TTGCCACTGACCAGGCCA | 425 |
| chr3 | 58141845 | 58141862 | +/− | 18 | TGCCACTGACCAGGCCAC | 426 |
| chr3 | 58141846 | 58141863 | +/− | 18 | GCCACTGACCAGGCCACA | 427 |
| chr3 | 58141847 | 58141864 | +/− | 18 | CCACTGACCAGGCCACAG | 428 |
| chr3 | 58141848 | 58141865 | +/− | 18 | CACTGACCAGGCCACAGA | 429 |
| chr3 | 58141849 | 58141866 | +/− | 18 | ACTGACCAGGCCACAGAT | 430 |
| chr3 | 58141850 | 58141867 | +/− | 18 | CTGACCAGGCCACAGATG | 431 |
| chr3 | 58141851 | 58141868 | +/− | 18 | TGACCAGGCCACAGATGG | 432 |
| chr3 | 58141852 | 58141869 | +/− | 18 | GACCAGGCCACAGATGGG | 433 |
| chr3 | 58141853 | 58141870 | +/− | 18 | ACCAGGCCACAGATGGGG | 434 |
| chr3 | 58141854 | 58141871 | +/− | 18 | CCAGGCCACAGATGGGGA | 435 |
| chr3 | 58141855 | 58141872 | +/− | 18 | CAGGCCACAGATGGGGAA | 436 |
| chr3 | 58141856 | 58141873 | +/− | 18 | AGGCCACAGATGGGGAAG | 437 |
| chr3 | 58141857 | 58141874 | +/− | 18 | GGCCACAGATGGGGAAGT | 438 |
| chr3 | 58141858 | 58141875 | +/− | 18 | GCCACAGATGGGGAAGTC | 439 |
| chr3 | 58141859 | 58141876 | +/− | 18 | CCACAGATGGGGAAGTCA | 440 |
| chr3 | 58141860 | 58141877 | +/− | 18 | CACAGATGGGGAAGTCAC | 441 |
| chr3 | 58141861 | 58141878 | +/− | 18 | ACAGATGGGGAAGTCACA | 442 |
| chr3 | 58141862 | 58141879 | +/− | 18 | CAGATGGGGAAGTCACAG | 443 |
| chr3 | 58141863 | 58141880 | +/− | 18 | AGATGGGGAAGTCACAGC | 444 |
| chr3 | 58141864 | 58141881 | +/− | 18 | GATGGGGAAGTCACAGCC | 445 |
| chr3 | 58141865 | 58141882 | +/− | 18 | ATGGGGAAGTCACAGCCG | 446 |
| chr3 | 58141866 | 58141883 | +/− | 18 | TGGGGAAGTCACAGCCGT | 447 |
| chr3 | 58141867 | 58141884 | +/− | 18 | GGGGAAGTCACAGCCGTG | 448 |
| chr3 | 58141868 | 58141885 | +/− | 18 | GGGAAGTCACAGCCGTGG | 449 |
| chr3 | 58141869 | 58141886 | +/− | 18 | GGAAGTCACAGCCGTGGA | 450 |
| chr3 | 58141870 | 58141887 | +/− | 18 | GAAGTCACAGCCGTGGAG | 451 |
| chr3 | 58141871 | 58141888 | +/− | 18 | AAGTCACAGCCGTGGAGG | 452 |
| chr3 | 58141872 | 58141889 | +/− | 18 | AGTCACAGCCGTGGAGGA | 453 |
| chr3 | 58141873 | 58141890 | +/− | 18 | GTCACAGCCGTGGAGGAG | 454 |
| chr3 | 58141874 | 58141891 | +/− | 18 | TCACAGCCGTGGAGGAGG | 455 |
| chr3 | 58141875 | 58141892 | +/− | 18 | CACAGCCGTGGAGGAGGC | 456 |
| chr3 | 58141876 | 58141893 | +/− | 18 | ACAGCCGTGGAGGAGGCA | 457 |
| chr3 | 58141877 | 58141894 | +/− | 18 | CAGCCGTGGAGGAGGCAC | 458 |
| chr3 | 58141878 | 58141895 | +/− | 18 | AGCCGTGGAGGAGGCACC | 459 |
| chr3 | 58141879 | 58141896 | +/− | 18 | GCCGTGGAGGAGGCACCG | 460 |
| chr3 | 58141880 | 58141897 | +/− | 18 | CCGTGGAGGAGGCACCGG | 461 |
| chr3 | 58141881 | 58141898 | +/− | 18 | CGTGGAGGAGGCACCGGT | 462 |
| chr3 | 58141882 | 58141899 | +/− | 18 | GTGGAGGAGGCACCGGTA | 463 |
| chr3 | 58141883 | 58141900 | +/− | 18 | TGGAGGAGGCACCGGTAA | 464 |
| chr3 | 58141884 | 58141901 | +/− | 18 | GGAGGAGGCACCGGTAAA | 465 |
| chr3 | 58141885 | 58141902 | +/− | 18 | GAGGAGGCACCGGTAAAT | 466 |
| chr3 | 58141886 | 58141903 | +/− | 18 | AGGAGGCACCGGTAAATG | 467 |
| chr3 | 58141887 | 58141904 | +/− | 18 | GGAGGCACCGGTAAATGC | 468 |
| chr3 | 58141888 | 58141905 | +/− | 18 | GAGGCACCGGTAAATGCA | 469 |
| chr3 | 58141889 | 58141906 | +/− | 18 | AGGCACCGGTAAATGCAT | 470 |
| chr3 | 58141890 | 58141907 | +/− | 18 | GGCACCGGTAAATGCATG | 471 |
| chr3 | 58141891 | 58141908 | +/− | 18 | GCACCGGTAAATGCATGT | 472 |
| chr3 | 58141892 | 58141909 | +/− | 18 | CACCGGTAAATGCATGTC | 473 |
| chr3 | 58141893 | 58141910 | +/− | 18 | ACCGGTAAATGCATGTCC | 474 |
| chr3 | 58141894 | 58141911 | +/− | 18 | CCGGTAAATGCATGTCCC | 475 |
| chr3 | 58141895 | 58141912 | +/− | 18 | CGGTAAATGCATGTCCCC | 476 |
| chr3 | 58141896 | 58141913 | +/− | 18 | GGTAAATGCATGTCCCCC | 477 |
| chr3 | 58141897 | 58141914 | +/− | 18 | GTAAATGCATGTCCCCCT | 478 |
| chr3 | 58141898 | 58141915 | +/− | 18 | TAAATGCATGTCCCCCTG | 479 |
| chr3 | 58141899 | 58141916 | +/− | 18 | AAATGCATGTCCCCCTGG | 480 |
| chr3 | 58141900 | 58141917 | +/− | 18 | AATGCATGTCCCCCTGGA | 481 |
| chr3 | 58141901 | 58141918 | +/− | 18 | ATGCATGTCCCCCTGGAT | 482 |
| chr3 | 58141902 | 58141919 | +/− | 18 | TGCATGTCCCCCTGGATT | 483 |
| chr3 | 58141903 | 58141920 | +/− | 18 | GCATGTCCCCCTGGATTC | 484 |
| chr3 | 58141904 | 58141921 | +/− | 18 | CATGTCCCCCTGGATTCA | 485 |
| chr3 | 58141905 | 58141922 | +/− | 18 | ATGTCCCCCTGGATTCAG | 486 |
| chr3 | 58141906 | 58141923 | +/− | 18 | TGTCCCCCTGGATTCAGG | 487 |
| chr3 | 58141907 | 58141924 | +/− | 18 | GTCCCCCTGGATTCAGGC | 488 |
| chr3 | 58141908 | 58141925 | +/− | 18 | TCCCCCTGGATTCAGGCC | 489 |
| chr3 | 58141909 | 58141926 | +/− | 18 | CCCCCTGGATTCAGGCCC | 490 |
| chr3 | 58141910 | 58141927 | +/− | 18 | CCCCTGGATTCAGGCCCT | 491 |
| chr3 | 58141911 | 58141928 | +/− | 18 | CCCTGGATTCAGGCCCTG | 492 |
| chr3 | 58141912 | 58141929 | +/− | 18 | CCTGGATTCAGGCCCTGG | 493 |
| chr3 | 58141913 | 58141930 | +/− | 18 | CTGGATTCAGGCCCTGGG | 494 |
| chr3 | 58141914 | 58141931 | +/− | 18 | TGGATTCAGGCCCTGGGT | 495 |
| chr3 | 58141915 | 58141932 | +/− | 18 | GGATTCAGGCCCTGGGTA | 496 |
| chr3 | 58141916 | 58141933 | +/− | 18 | GATTCAGGCCCTGGGTAC | 497 |
| chr3 | 58141917 | 58141934 | +/− | 18 | ATTCAGGCCCTGGGTACA | 498 |
| chr3 | 58141918 | 58141935 | +/− | 18 | TTCAGGCCCTGGGTACAA | 499 |
| chr3 | 58141919 | 58141936 | +/− | 18 | TCAGGCCCTGGGTACAAT | 500 |
| chr3 | 58141920 | 58141937 | +/− | 18 | CAGGCCCTGGGTACAATT | 501 |
| chr3 | 58141921 | 58141938 | +/− | 18 | AGGCCCTGGGTACAATTT | 502 |
| chr3 | 58141922 | 58141939 | +/− | 18 | GGCCCTGGGTACAATTTT | 503 |
| chr3 | 58141923 | 58141940 | +/− | 18 | GCCCTGGGTACAATTTTG | 504 |
| chr3 | 58141924 | 58141941 | +/− | 18 | CCCTGGGTACAATTTTGG | 505 |
| chr3 | 58141925 | 58141942 | +/− | 18 | CCTGGGTACAATTTTGGT | 506 |
| chr3 | 58141926 | 58141943 | +/− | 18 | CTGGGTACAATTTTGGTT | 507 |
| chr3 | 58141927 | 58141944 | +/− | 18 | TGGGTACAATTTTGGTTT | 508 |
| chr3 | 58141928 | 58141945 | +/− | 18 | GGGTACAATTTTGGTTTT | 509 |
| chr3 | 58141929 | 58141946 | +/− | 18 | GGTACAATTTTGGTTTTT | 510 |
| chr3 | 58141930 | 58141947 | +/− | 18 | GTACAATTTTGGTTTTTT | 511 |
| chr3 | 58141931 | 58141948 | +/− | 18 | TACAATTTTGGTTTTTTC | 512 |
| chr3 | 58141932 | 58141949 | +/− | 18 | ACAATTTTGGTTTTTTCC | 513 |
| chr3 | 58141933 | 58141950 | +/− | 18 | CAATTTTGGTTTTTTCCT | 514 |
| chr3 | 58141934 | 58141951 | +/− | 18 | AATTTTGGTTTTTTCCTT | 515 |
| chr3 | 58141935 | 58141952 | +/− | 18 | ATTTTGGTTTTTTCCTTT | 516 |
| chr3 | 58141936 | 58141953 | +/− | 18 | TTTTGGTTTTTTCCTTTT | 517 |
| chr3 | 58141937 | 58141954 | +/− | 18 | TTTGGTTTTTTCCTTTTT | 518 |
| chr3 | 58141938 | 58141955 | +/− | 18 | TTGGTTTTTTCCTTTTTG | 519 |
| chr3 | 58141939 | 58141956 | +/− | 18 | TGGTTTTTTCCTTTTTGT | 520 |
| chr3 | 58141940 | 58141957 | +/− | 18 | GGTTTTTTCCTTTTTGTG | 521 |
| chr3 | 58141941 | 58141958 | +/− | 18 | GTTTTTTCCTTTTTGTGT | 522 |
| chr3 | 58141942 | 58141959 | +/− | 18 | TTTTTTCCTTTTTGTGTT | 523 |
| chr3 | 58141943 | 58141960 | +/− | 18 | TTTTTCCTTTTTGTGTTT | 524 |
| chr3 | 58141944 | 58141961 | +/− | 18 | TTTTCCTTTTTGTGTTTC | 525 |
| chr3 | 58141945 | 58141962 | +/− | 18 | TTTCCTTTTTGTGTTTCT | 526 |
| chr3 | 58141946 | 58141963 | +/− | 18 | TTCCTTTTTGTGTTTCTG | 527 |
| chr3 | 58141947 | 58141964 | +/− | 18 | TCCTTTTTGTGTTTCTGT | 528 |
| chr3 | 58141948 | 58141965 | +/− | 18 | CCTTTTTGTGTTTCTGTG | 529 |
| chr3 | 58141949 | 58141966 | +/− | 18 | CTTTTTGTGTTTCTGTGT | 530 |
| chr3 | 58141950 | 58141967 | +/− | 18 | TTTTTGTGTTTCTGTGTT | 531 |
| chr3 | 58141951 | 58141968 | +/− | 18 | TTTTGTGTTTCTGTGTTT | 532 |
| chr3 | 58141952 | 58141969 | +/− | 18 | TTTGTGTTTCTGTGTTTA | 533 |
| chr3 | 58141953 | 58141970 | +/− | 18 | TTGTGTTTCTGTGTTTAC | 534 |
| chr3 | 58141954 | 58141971 | +/− | 18 | TGTGTTTCTGTGTTTACT | 535 |
| chr3 | 58141955 | 58141972 | +/− | 18 | GTGTTTCTGTGTTTACTC | 536 |
| chr3 | 58141956 | 58141973 | +/− | 18 | TGTTTCTGTGTTTACTCA | 537 |
| chr3 | 58141957 | 58141974 | +/− | 18 | GTTTCTGTGTTTACTCAG | 538 |
| chr3 | 58141958 | 58141975 | +/− | 18 | TTTCTGTGTTTACTCAGC | 539 |
| chr3 | 58141959 | 58141976 | +/− | 18 | TTCTGTGTTTACTCAGCC | 540 |
| chr3 | 58141960 | 58141977 | +/− | 18 | TCTGTGTTTACTCAGCCT | 541 |
| chr3 | 58141961 | 58141978 | +/− | 18 | CTGTGTTTACTCAGCCTT | 542 |
| chr3 | 58141962 | 58141979 | +/− | 18 | TGTGTTTACTCAGCCTTC | 543 |
| chr3 | 58141963 | 58141980 | +/− | 18 | GTGTTTACTCAGCCTTCA | 544 |
| chr3 | 58141964 | 58141981 | +/− | 18 | TGTTTACTCAGCCTTCAT | 545 |
| chr3 | 58141965 | 58141982 | +/− | 18 | GTTTACTCAGCCTTCATT | 546 |
| chr3 | 58141966 | 58141983 | +/− | 18 | TTTACTCAGCCTTCATTT | 547 |
| chr3 | 58141967 | 58141984 | +/− | 18 | TTACTCAGCCTTCATTTC | 548 |
| chr3 | 58141968 | 58141985 | +/− | 18 | TACTCAGCCTTCATTTCA | 549 |
| chr3 | 58141969 | 58141986 | +/− | 18 | ACTCAGCCTTCATTTCAG | 550 |
| chr3 | 58141970 | 58141987 | +/− | 18 | CTCAGCCTTCATTTCAGA | 551 |
| chr3 | 58141971 | 58141988 | +/− | 18 | TCAGCCTTCATTTCAGAA | 552 |
| chr3 | 58141972 | 58141989 | +/− | 18 | CAGCCTTCATTTCAGAAA | 553 |
| chr3 | 58141973 | 58141990 | +/− | 18 | AGCCTTCATTTCAGAAAA | 554 |
| chr3 | 58141974 | 58141991 | +/− | 18 | GCCTTCATTTCAGAAAAT | 555 |
| chr3 | 58141975 | 58141992 | +/− | 18 | CCTTCATTTCAGAAAATC | 556 |
| chr3 | 58141976 | 58141993 | +/− | 18 | CTTCATTTCAGAAAATCT | 557 |
| chr3 | 58141977 | 58141994 | +/− | 18 | TTCATTTCAGAAAATCTG | 558 |
| chr3 | 58141978 | 58141995 | +/− | 18 | TCATTTCAGAAAATCTGC | 559 |
| chr3 | 58141979 | 58141996 | +/− | 18 | CATTTCAGAAAATCTGCC | 560 |
| chr3 | 58141980 | 58141997 | +/− | 18 | ATTTCAGAAAATCTGCCA | 561 |
| chr3 | 58141981 | 58141998 | +/− | 18 | TTTCAGAAAATCTGCCAT | 562 |
| chr3 | 58141982 | 58141999 | +/− | 18 | TTCAGAAAATCTGCCATC | 563 |
| chr3 | 58141983 | 58142000 | +/− | 18 | TCAGAAAATCTGCCATCT | 564 |
| chr3 | 58141984 | 58142001 | +/− | 18 | CAGAAAATCTGCCATCTG | 565 |
| chr3 | 58141985 | 58142002 | +/− | 18 | AGAAAATCTGCCATCTGC | 566 |
| chr3 | 58141986 | 58142003 | +/− | 18 | GAAAATCTGCCATCTGCT | 567 |
| chr3 | 58141987 | 58142004 | +/− | 18 | AAAATCTGCCATCTGCTT | 568 |
| chr3 | 58141988 | 58142005 | +/− | 18 | AAATCTGCCATCTGCTTC | 569 |
| chr3 | 58141989 | 58142006 | +/− | 18 | AATCTGCCATCTGCTTCT | 570 |
| chr3 | 58141990 | 58142007 | +/− | 18 | ATCTGCCATCTGCTTCTG | 571 |
| chr3 | 58141991 | 58142008 | +/− | 18 | TCTGCCATCTGCTTCTGG | 572 |
| chr3 | 58141992 | 58142009 | +/− | 18 | CTGCCATCTGCTTCTGGG | 573 |
| chr3 | 58141993 | 58142010 | +/− | 18 | TGCCATCTGCTTCTGGGA | 574 |
| chr3 | 58141994 | 58142011 | +/− | 18 | GCCATCTGCTTCTGGGAT | 575 |
| chr3 | 58141995 | 58142012 | +/− | 18 | CCATCTGCTTCTGGGATT | 576 |
| chr3 | 58141996 | 58142013 | +/− | 18 | CATCTGCTTCTGGGATTG | 577 |
| chr3 | 58141997 | 58142014 | +/− | 18 | ATCTGCTTCTGGGATTGC | 578 |
| chr3 | 58141998 | 58142015 | +/− | 18 | TCTGCTTCTGGGATTGCT | 579 |
| chr3 | 58141999 | 58142016 | +/− | 18 | CTGCTTCTGGGATTGCTT | 580 |
| chr3 | 58142000 | 58142017 | +/− | 18 | TGCTTCTGGGATTGCTTA | 581 |
| chr3 | 58142001 | 58142018 | +/− | 18 | GCTTCTGGGATTGCTTAA | 582 |
| chr3 | 58142002 | 58142019 | +/− | 18 | CTTCTGGGATTGCTTAAG | 583 |
| chr3 | 58142003 | 58142020 | +/− | 18 | TTCTGGGATTGCTTAAGC | 584 |
| chr3 | 58142004 | 58142021 | +/− | 18 | TCTGGGATTGCTTAAGCC | 585 |
| chr3 | 58142005 | 58142022 | +/− | 18 | CTGGGATTGCTTAAGCCC | 586 |
| chr3 | 58142006 | 58142023 | +/− | 18 | TGGGATTGCTTAAGCCCT | 587 |
| chr3 | 58142007 | 58142024 | +/− | 18 | GGGATTGCTTAAGCCCTG | 588 |
| chr3 | 58142008 | 58142025 | +/− | 18 | GGATTGCTTAAGCCCTGT | 589 |
| chr3 | 58142009 | 58142026 | +/− | 18 | GATTGCTTAAGCCCTGTG | 590 |
| chr3 | 58142010 | 58142027 | +/− | 18 | ATTGCTTAAGCCCTGTGG | 591 |
| chr3 | 58142011 | 58142028 | +/− | 18 | TTGCTTAAGCCCTGTGGG | 592 |
| chr3 | 58142012 | 58142029 | +/− | 18 | TGCTTAAGCCCTGTGGGT | 593 |
| chr3 | 58142013 | 58142030 | +/− | 18 | GCTTAAGCCCTGTGGGTG | 594 |
| chr3 | 58142014 | 58142031 | +/− | 18 | CTTAAGCCCTGTGGGTGT | 595 |
| chr3 | 58142015 | 58142032 | +/− | 18 | TTAAGCCCTGTGGGTGTC | 596 |
| chr3 | 58142016 | 58142033 | +/− | 18 | TAAGCCCTGTGGGTGTCC | 597 |
| chr3 | 58142017 | 58142034 | +/− | 18 | AAGCCCTGTGGGTGTCCT | 598 |
| chr3 | 58142018 | 58142035 | +/− | 18 | AGCCCTGTGGGTGTCCTG | 599 |
| chr3 | 58142019 | 58142036 | +/− | 18 | GCCCTGTGGGTGTCCTGG | 600 |
| chr3 | 58142020 | 58142037 | +/− | 18 | CCCTGTGGGTGTCCTGGT | 601 |
| chr3 | 58142021 | 58142038 | +/− | 18 | CCTGTGGGTGTCCTGGTC | 602 |
| chr3 | 58142022 | 58142039 | +/− | 18 | CTGTGGGTGTCCTGGTCA | 603 |
| chr3 | 58142023 | 58142040 | +/− | 18 | TGTGGGTGTCCTGGTCAT | 604 |
| chr3 | 58142024 | 58142041 | +/− | 18 | GTGGGTGTCCTGGTCATT | 605 |
| chr3 | 58142025 | 58142042 | +/− | 18 | TGGGTGTCCTGGTCATTG | 606 |
| chr3 | 58142026 | 58142043 | +/− | 18 | GGGTGTCCTGGTCATTGG | 607 |
| chr3 | 58142027 | 58142044 | +/− | 18 | GGTGTCCTGGTCATTGGT | 608 |

TABLE 5
19-mer target-specific ASOs

| | | | | | | SEQ |
|---|---|---|---|---|---|---|
| | | | | | | ID |
| CHR | START | END | STRAND | kmer | SEQUENCE | NO: |
| chr3 | 58141828 | 58141846 | +/− | 19 | CCCAACTAATCTCCATTTG | 609 |
| chr3 | 58141829 | 58141847 | +/− | 19 | CCAACTAATCTCCATTTGC | 610 |
| chr3 | 58141830 | 58141848 | +/− | 19 | CAACTAATCTCCATTTGCC | 611 |
| chr3 | 58141831 | 58141849 | +/− | 19 | AACTAATCTCCATTTGCCA | 612 |
| chr3 | 58141832 | 58141850 | +/− | 19 | ACTAATCTCCATTTGCCAC | 613 |
| chr3 | 58141833 | 58141851 | +/− | 19 | CTAATCTCCATTTGCCACT | 614 |
| chr3 | 58141834 | 58141852 | +/− | 19 | TAATCTCCATTTGCCACTG | 615 |
| chr3 | 58141835 | 58141853 | +/− | 19 | AATCTCCATTTGCCACTGA | 616 |
| chr3 | 58141836 | 58141854 | +/− | 19 | ATCTCCATTTGCCACTGAC | 617 |
| chr3 | 58141837 | 58141855 | +/− | 19 | TCTCCATTTGCCACTGACC | 618 |
| chr3 | 58141838 | 58141856 | +/− | 19 | CTCCATTTGCCACTGACCA | 619 |
| chr3 | 58141839 | 58141857 | +/− | 19 | TCCATTTGCCACTGACCAG | 620 |
| chr3 | 58141840 | 58141858 | +/− | 19 | CCATTTGCCACTGACCAGG | 621 |
| chr3 | 58141841 | 58141859 | +/− | 19 | CATTTGCCACTGACCAGGC | 622 |
| chr3 | 58141842 | 58141860 | +/− | 19 | ATTTGCCACTGACCAGGCC | 623 |
| chr3 | 58141843 | 58141861 | +/− | 19 | TTTGCCACTGACCAGGCCA | 624 |
| chr3 | 58141844 | 58141862 | +/− | 19 | TTGCCACTGACCAGGCCAC | 625 |
| chr3 | 58141845 | 58141863 | +/− | 19 | TGCCACTGACCAGGCCACA | 626 |
| chr3 | 58141846 | 58141864 | +/− | 19 | GCCACTGACCAGGCCACAG | 627 |
| chr3 | 58141847 | 58141865 | +/− | 19 | CCACTGACCAGGCCACAGA | 628 |
| chr3 | 58141848 | 58141866 | +/− | 19 | CACTGACCAGGCCACAGAT | 629 |
| chr3 | 58141849 | 58141867 | +/− | 19 | ACTGACCAGGCCACAGATG | 630 |
| chr3 | 58141850 | 58141868 | +/− | 19 | CTGACCAGGCCACAGATGG | 631 |
| chr3 | 58141851 | 58141869 | +/− | 19 | TGACCAGGCCACAGATGGG | 632 |
| chr3 | 58141852 | 58141870 | +/− | 19 | GACCAGGCCACAGATGGGG | 633 |
| chr3 | 58141853 | 58141871 | +/− | 19 | ACCAGGCCACAGATGGGGA | 634 |
| chr3 | 58141854 | 58141872 | +/− | 19 | CCAGGCCACAGATGGGGAA | 635 |
| chr3 | 58141855 | 58141873 | +/− | 19 | CAGGCCACAGATGGGGAAG | 636 |
| chr3 | 58141856 | 58141874 | +/− | 19 | AGGCCACAGATGGGGAAGT | 637 |
| chr3 | 58141857 | 58141875 | +/− | 19 | GGCCACAGATGGGGAAGTC | 638 |
| chr3 | 58141858 | 58141876 | +/− | 19 | GCCACAGATGGGGAAGTCA | 639 |
| chr3 | 58141859 | 58141877 | +/− | 19 | CCACAGATGGGGAAGTCAC | 640 |
| chr3 | 58141860 | 58141878 | +/− | 19 | CACAGATGGGGAAGTCACA | 641 |
| chr3 | 58141861 | 58141879 | +/− | 19 | ACAGATGGGGAAGTCACAG | 642 |
| chr3 | 58141862 | 58141880 | +/− | 19 | CAGATGGGGAAGTCACAGC | 643 |
| chr3 | 58141863 | 58141881 | +/− | 19 | AGATGGGGAAGTCACAGCC | 644 |
| chr3 | 58141864 | 58141882 | +/− | 19 | GATGGGGAAGTCACAGCCG | 645 |
| chr3 | 58141865 | 58141883 | +/− | 19 | ATGGGGAAGTCACAGCCGT | 646 |
| chr3 | 58141866 | 58141884 | +/− | 19 | TGGGGAAGTCACAGCCGTG | 647 |
| chr3 | 58141867 | 58141885 | +/− | 19 | GGGGAAGTCACAGCCGTGG | 648 |
| chr3 | 58141868 | 58141886 | +/− | 19 | GGGAAGTCACAGCCGTGGA | 649 |
| chr3 | 58141869 | 58141887 | +/− | 19 | GGAAGTCACAGCCGTGGAG | 650 |
| chr3 | 58141870 | 58141888 | +/− | 19 | GAAGTCACAGCCGTGGAGG | 651 |
| chr3 | 58141871 | 58141889 | +/− | 19 | AAGTCACAGCCGTGGAGGA | 652 |
| chr3 | 58141872 | 58141890 | +/− | 19 | AGTCACAGCCGTGGAGGAG | 653 |
| chr3 | 58141873 | 58141891 | +/− | 19 | GTCACAGCCGTGGAGGAGG | 654 |
| chr3 | 58141874 | 58141892 | +/− | 19 | TCACAGCCGTGGAGGAGGC | 655 |
| chr3 | 58141875 | 58141893 | +/− | 19 | CACAGCCGTGGAGGAGGCA | 656 |
| chr3 | 58141876 | 58141894 | +/− | 19 | ACAGCCGTGGAGGAGGCAC | 657 |
| chr3 | 58141877 | 58141895 | +/− | 19 | CAGCCGTGGAGGAGGCACC | 658 |
| chr3 | 58141878 | 58141896 | +/− | 19 | AGCCGTGGAGGAGGCACCG | 659 |
| chr3 | 58141879 | 58141897 | +/− | 19 | GCCGTGGAGGAGGCACCGG | 660 |
| chr3 | 58141880 | 58141898 | +/− | 19 | CCGTGGAGGAGGCACCGGT | 661 |
| chr3 | 58141881 | 58141899 | +/− | 19 | CGTGGAGGAGGCACCGGTA | 662 |
| chr3 | 58141882 | 58141900 | +/− | 19 | GTGGAGGAGGCACCGGTAA | 663 |
| chr3 | 58141883 | 58141901 | +/− | 19 | TGGAGGAGGCACCGGTAAA | 664 |
| chr3 | 58141884 | 58141902 | +/− | 19 | GGAGGAGGCACCGGTAAAT | 665 |
| chr3 | 58141885 | 58141903 | +/− | 19 | GAGGAGGCACCGGTAAATG | 666 |
| chr3 | 58141886 | 58141904 | +/− | 19 | AGGAGGCACCGGTAAATGC | 667 |
| chr3 | 58141887 | 58141905 | +/− | 19 | GGAGGCACCGGTAAATGCA | 668 |
| chr3 | 58141888 | 58141906 | +/− | 19 | GAGGCACCGGTAAATGCAT | 669 |
| chr3 | 58141889 | 58141907 | +/− | 19 | AGGCACCGGTAAATGCATG | 670 |
| chr3 | 58141890 | 58141908 | +/− | 19 | GGCACCGGTAAATGCATGT | 671 |
| chr3 | 58141891 | 58141909 | +/− | 19 | GCACCGGTAAATGCATGTC | 672 |
| chr3 | 58141892 | 58141910 | +/− | 19 | CACCGGTAAATGCATGTCC | 673 |
| chr3 | 58141893 | 58141911 | +/− | 19 | ACCGGTAAATGCATGTCCC | 674 |
| chr3 | 58141894 | 58141912 | +/− | 19 | CCGGTAAATGCATGTCCCC | 675 |
| chr3 | 58141895 | 58141913 | +/− | 19 | CGGTAAATGCATGTCCCCC | 676 |
| chr3 | 58141896 | 58141914 | +/− | 19 | GGTAAATGCATGTCCCCCT | 677 |
| chr3 | 58141897 | 58141915 | +/− | 19 | GTAAATGCATGTCCCCCTG | 678 |
| chr3 | 58141898 | 58141916 | +/− | 19 | TAAATGCATGTCCCCCTGG | 679 |
| chr3 | 58141899 | 58141917 | +/− | 19 | AAATGCATGTCCCCCTGGA | 680 |
| chr3 | 58141900 | 58141918 | +/− | 19 | AATGCATGTCCCCCTGGAT | 681 |
| chr3 | 58141901 | 58141919 | +/− | 19 | ATGCATGTCCCCCTGGATT | 682 |
| chr3 | 58141902 | 58141920 | +/− | 19 | TGCATGTCCCCCTGGATTC | 683 |
| chr3 | 58141903 | 58141921 | +/− | 19 | GCATGTCCCCCTGGATTCA | 684 |
| chr3 | 58141904 | 58141922 | +/− | 19 | CATGTCCCCCTGGATTCAG | 685 |
| chr3 | 58141905 | 58141923 | +/− | 19 | ATGTCCCCCTGGATTCAGG | 686 |
| chr3 | 58141906 | 58141924 | +/− | 19 | TGTCCCCCTGGATTCAGGC | 687 |
| chr3 | 58141907 | 58141925 | +/− | 19 | GTCCCCCTGGATTCAGGCC | 688 |
| chr3 | 58141908 | 58141926 | +/− | 19 | TCCCCCTGGATTCAGGCCC | 689 |
| chr3 | 58141909 | 58141927 | +/− | 19 | CCCCCTGGATTCAGGCCCT | 690 |
| chr3 | 58141910 | 58141928 | +/− | 19 | CCCCTGGATTCAGGCCCTG | 691 |
| chr3 | 58141911 | 58141929 | +/− | 19 | CCCTGGATTCAGGCCCTGG | 692 |
| chr3 | 58141912 | 58141930 | +/− | 19 | CCTGGATTCAGGCCCTGGG | 693 |
| chr3 | 58141913 | 58141931 | +/− | 19 | CTGGATTCAGGCCCTGGGT | 694 |
| chr3 | 58141914 | 58141932 | +/− | 19 | TGGATTCAGGCCCTGGGTA | 695 |
| chr3 | 58141915 | 58141933 | +/− | 19 | GGATTCAGGCCCTGGGTAC | 696 |
| chr3 | 58141916 | 58141934 | +/− | 19 | GATTCAGGCCCTGGGTACA | 697 |
| chr3 | 58141917 | 58141935 | +/− | 19 | ATTCAGGCCCTGGGTACAA | 698 |
| chr3 | 58141918 | 58141936 | +/− | 19 | TTCAGGCCCTGGGTACAAT | 699 |
| chr3 | 58141919 | 58141937 | +/− | 19 | TCAGGCCCTGGGTACAATT | 700 |
| chr3 | 58141920 | 58141938 | +/− | 19 | CAGGCCCTGGGTACAATTT | 701 |
| chr3 | 58141921 | 58141939 | +/− | 19 | AGGCCCTGGGTACAATTTT | 702 |
| chr3 | 58141922 | 58141940 | +/− | 19 | GGCCCTGGGTACAATTTTG | 703 |
| chr3 | 58141923 | 58141941 | +/− | 19 | GCCCTGGGTACAATTTTGG | 704 |
| chr3 | 58141924 | 58141942 | +/− | 19 | CCCTGGGTACAATTTTGGT | 705 |
| chr3 | 58141925 | 58141943 | +/− | 19 | CCTGGGTACAATTTTGGTT | 706 |
| chr3 | 58141926 | 58141944 | +/− | 19 | CTGGGTACAATTTTGGTTT | 707 |
| chr3 | 58141927 | 58141945 | +/− | 19 | TGGGTACAATTTTGGTTTT | 708 |
| chr3 | 58141928 | 58141946 | +/− | 19 | GGGTACAATTTTGGTTTTT | 709 |
| chr3 | 58141929 | 58141947 | +/− | 19 | GGTACAATTTTGGTTTTTT | 710 |
| chr3 | 58141930 | 58141948 | +/− | 19 | GTACAATTTTGGTTTTTTC | 711 |
| chr3 | 58141931 | 58141949 | +/− | 19 | TACAATTTTGGTTTTTTCC | 712 |
| chr3 | 58141932 | 58141950 | +/− | 19 | ACAATTTTGGTTTTTTCCT | 713 |
| chr3 | 58141933 | 58141951 | +/− | 19 | CAATTTTGGTTTTTTCCTT | 714 |
| chr3 | 58141934 | 58141952 | +/− | 19 | AATTTTGGTTTTTTCCTTT | 715 |
| chr3 | 58141935 | 58141953 | +/− | 19 | ATTTTGGTTTTTTCCTTTT | 716 |
| chr3 | 58141936 | 58141954 | +/− | 19 | TTTTGGTTTTTTCCTTTTT | 717 |
| chr3 | 58141937 | 58141955 | +/− | 19 | TTTGGTTTTTTCCTTTTTG | 718 |
| chr3 | 58141938 | 58141956 | +/− | 19 | TTGGTTTTTTCCTTTTTGT | 719 |
| chr3 | 58141939 | 58141957 | +/− | 19 | TGGTTTTTTCCTTTTTGTG | 720 |
| chr3 | 58141940 | 58141958 | +/− | 19 | GGTTTTTTCCTTTTTGTGT | 721 |
| chr3 | 58141941 | 58141959 | +/− | 19 | GTTTTTTCCTTTTTGTGTT | 722 |
| chr3 | 58141942 | 58141960 | +/− | 19 | TTTTTTCCTTTTTGTGTTT | 723 |
| chr3 | 58141943 | 58141961 | +/− | 19 | TTTTTCCTTTTTGTGTTTC | 724 |
| chr3 | 58141944 | 58141962 | +/− | 19 | TTTTCCTTTTTGTGTTTCT | 725 |
| chr3 | 58141945 | 58141963 | +/− | 19 | TTTCCTTTTTGTGTTTCTG | 726 |
| chr3 | 58141946 | 58141964 | +/− | 19 | TTCCTTTTTGTGTTTCTGT | 727 |
| chr3 | 58141947 | 58141965 | +/− | 19 | TCCTTTTTGTGTTTCTGTG | 728 |
| chr3 | 58141948 | 58141966 | +/− | 19 | CCTTTTTGTGTTTCTGTGT | 729 |
| chr3 | 58141949 | 58141967 | +/− | 19 | CTTTTTGTGTTTCTGTGTT | 730 |
| chr3 | 58141950 | 58141968 | +/− | 19 | TTTTTGTGTTTCTGTGTTT | 731 |
| chr3 | 58141951 | 58141969 | +/− | 19 | TTTTGTGTTTCTGTGTTTA | 732 |
| chr3 | 58141952 | 58141970 | +/− | 19 | TTTGTGTTTCTGTGTTTAC | 733 |
| chr3 | 58141953 | 58141971 | +/− | 19 | TTGTGTTTCTGTGTTTACT | 734 |
| chr3 | 58141954 | 58141972 | +/− | 19 | TGTGTTTCTGTGTTTACTC | 735 |
| chr3 | 58141955 | 58141973 | +/− | 19 | GTGTTTCTGTGTTTACTCA | 736 |
| chr3 | 58141956 | 58141974 | +/− | 19 | TGTTTCTGTGTTTACTCAG | 737 |
| chr3 | 58141957 | 58141975 | +/− | 19 | GTTTCTGTGTTTACTCAGC | 738 |
| chr3 | 58141958 | 58141976 | +/− | 19 | TTTCTGTGTTTACTCAGCC | 739 |
| chr3 | 58141959 | 58141977 | +/− | 19 | TTCTGTGTTTACTCAGCCT | 740 |
| chr3 | 58141960 | 58141978 | +/− | 19 | TCTGTGTTTACTCAGCCTT | 741 |
| chr3 | 58141961 | 58141979 | +/− | 19 | CTGTGTTTACTCAGCCTTC | 742 |
| chr3 | 58141962 | 58141980 | +/− | 19 | TGTGTTTACTCAGCCTTCA | 743 |
| chr3 | 58141963 | 58141981 | +/− | 19 | GTGTTTACTCAGCCTTCAT | 744 |
| chr3 | 58141964 | 58141982 | +/− | 19 | TGTTTACTCAGCCTTCATT | 745 |
| chr3 | 58141965 | 58141983 | +/− | 19 | GTTTACTCAGCCTTCATTT | 746 |
| chr3 | 58141966 | 58141984 | +/− | 19 | TTTACTCAGCCTTCATTTC | 747 |
| chr3 | 58141967 | 58141985 | +/− | 19 | TTACTCAGCCTTCATTTCA | 748 |
| chr3 | 58141968 | 58141986 | +/− | 19 | TACTCAGCCTTCATTTCAG | 749 |
| chr3 | 58141969 | 58141987 | +/− | 19 | ACTCAGCCTTCATTTCAGA | 750 |
| chr3 | 58141970 | 58141988 | +/− | 19 | CTCAGCCTTCATTTCAGAA | 751 |
| chr3 | 58141971 | 58141989 | +/− | 19 | TCAGCCTTCATTTCAGAAA | 752 |
| chr3 | 58141972 | 58141990 | +/− | 19 | CAGCCTTCATTTCAGAAAA | 753 |
| chr3 | 58141973 | 58141991 | +/− | 19 | AGCCTTCATTTCAGAAAAT | 754 |
| chr3 | 58141974 | 58141992 | +/− | 19 | GCCTTCATTTCAGAAAATC | 755 |
| chr3 | 58141975 | 58141993 | +/− | 19 | CCTTCATTTCAGAAAATCT | 756 |
| chr3 | 58141976 | 58141994 | +/− | 19 | CTTCATTTCAGAAAATCTG | 757 |
| chr3 | 58141977 | 58141995 | +/− | 19 | TTCATTTCAGAAAATCTGC | 758 |
| chr3 | 58141978 | 58141996 | +/− | 19 | TCATTTCAGAAAATCTGCC | 759 |
| chr3 | 58141979 | 58141997 | +/− | 19 | CATTTCAGAAAATCTGCCA | 760 |
| chr3 | 58141980 | 58141998 | +/− | 19 | ATTTCAGAAAATCTGCCAT | 761 |
| chr3 | 58141981 | 58141999 | +/− | 19 | TTTCAGAAAATCTGCCATC | 762 |
| chr3 | 58141982 | 58142000 | +/− | 19 | TTCAGAAAATCTGCCATCT | 763 |
| chr3 | 58141983 | 58142001 | +/− | 19 | TCAGAAAATCTGCCATCTG | 764 |
| chr3 | 58141984 | 58142002 | +/− | 19 | CAGAAAATCTGCCATCTGC | 765 |
| chr3 | 58141985 | 58142003 | +/− | 19 | AGAAAATCTGCCATCTGCT | 766 |
| chr3 | 58141986 | 58142004 | +/− | 19 | GAAAATCTGCCATCTGCTT | 767 |
| chr3 | 58141987 | 58142005 | +/− | 19 | AAAATCTGCCATCTGCTTC | 768 |
| chr3 | 58141988 | 58142006 | +/− | 19 | AAATCTGCCATCTGCTTCT | 769 |
| chr3 | 58141989 | 58142007 | +/− | 19 | AATCTGCCATCTGCTTCTG | 770 |
| chr3 | 58141990 | 58142008 | +/− | 19 | ATCTGCCATCTGCTTCTGG | 771 |
| chr3 | 58141991 | 58142009 | +/− | 19 | TCTGCCATCTGCTTCTGGG | 772 |
| chr3 | 58141992 | 58142010 | +/− | 19 | CTGCCATCTGCTTCTGGGA | 773 |
| chr3 | 58141993 | 58142011 | +/− | 19 | TGCCATCTGCTTCTGGGAT | 774 |
| chr3 | 58141994 | 58142012 | +/− | 19 | GCCATCTGCTTCTGGGATT | 775 |
| chr3 | 58141995 | 58142013 | +/− | 19 | CCATCTGCTTCTGGGATTG | 776 |
| chr3 | 58141996 | 58142014 | +/− | 19 | CATCTGCTTCTGGGATTGC | 777 |
| chr3 | 58141997 | 58142015 | +/− | 19 | ATCTGCTTCTGGGATTGCT | 778 |
| chr3 | 58141998 | 58142016 | +/− | 19 | TCTGCTTCTGGGATTGCTT | 779 |
| chr3 | 58141999 | 58142017 | +/− | 19 | CTGCTTCTGGGATTGCTTA | 780 |
| chr3 | 58142000 | 58142018 | +/− | 19 | TGCTTCTGGGATTGCTTAA | 781 |
| chr3 | 58142001 | 58142019 | +/− | 19 | GCTTCTGGGATTGCTTAAG | 782 |
| chr3 | 58142002 | 58142020 | +/− | 19 | CTTCTGGGATTGCTTAAGC | 783 |
| chr3 | 58142003 | 58142021 | +/− | 19 | TTCTGGGATTGCTTAAGCC | 784 |
| chr3 | 58142004 | 58142022 | +/− | 19 | TCTGGGATTGCTTAAGCCC | 785 |
| chr3 | 58142005 | 58142023 | +/− | 19 | CTGGGATTGCTTAAGCCCT | 786 |
| chr3 | 58142006 | 58142024 | +/− | 19 | TGGGATTGCTTAAGCCCTG | 787 |
| chr3 | 58142007 | 58142025 | +/− | 19 | GGGATTGCTTAAGCCCTGT | 788 |
| chr3 | 58142008 | 58142026 | +/− | 19 | GGATTGCTTAAGCCCTGTG | 789 |
| chr3 | 58142009 | 58142027 | +/− | 19 | GATTGCTTAAGCCCTGTGG | 790 |
| chr3 | 58142010 | 58142028 | +/− | 19 | ATTGCTTAAGCCCTGTGGG | 791 |
| chr3 | 58142011 | 58142029 | +/− | 19 | TTGCTTAAGCCCTGTGGGT | 792 |
| chr3 | 58142012 | 58142030 | +/− | 19 | TGCTTAAGCCCTGTGGGTG | 793 |
| chr3 | 58142013 | 58142031 | +/− | 19 | GCTTAAGCCCTGTGGGTGT | 794 |
| chr3 | 58142014 | 58142032 | +/− | 19 | CTTAAGCCCTGTGGGTGTC | 795 |
| chr3 | 58142015 | 58142033 | +/− | 19 | TTAAGCCCTGTGGGTGTCC | 796 |
| chr3 | 58142016 | 58142034 | +/− | 19 | TAAGCCCTGTGGGTGTCCT | 797 |
| chr3 | 58142017 | 58142035 | +/− | 19 | AAGCCCTGTGGGTGTCCTG | 798 |
| chr3 | 58142018 | 58142036 | +/− | 19 | AGCCCTGTGGGTGTCCTGG | 799 |
| chr3 | 58142019 | 58142037 | +/− | 19 | GCCCTGTGGGTGTCCTGGT | 800 |
| chr3 | 58142020 | 58142038 | +/− | 19 | CCCTGTGGGTGTCCTGGTC | 801 |
| chr3 | 58142021 | 58142039 | +/− | 19 | CCTGTGGGTGTCCTGGTCA | 802 |
| chr3 | 58142022 | 58142040 | +/− | 19 | CTGTGGGTGTCCTGGTCAT | 803 |
| chr3 | 58142023 | 58142041 | +/− | 19 | TGTGGGTGTCCTGGTCATT | 804 |
| chr3 | 58142024 | 58142042 | +/− | 19 | GTGGGTGTCCTGGTCATTG | 805 |
| chr3 | 58142025 | 58142043 | +/− | 19 | TGGGTGTCCTGGTCATTGG | 806 |
| chr3 | 58142026 | 58142044 | +/− | 19 | GGGTGTCCTGGTCATTGGT | 807 |

TABLE 6
20-mer target-specific ASOs

| | | | | | | SEQ |
|---|---|---|---|---|---|---|
| | | | | | | ID |
| CHR | START | END | STRAND | kmer | SEQUENCE | NO: |
| chr3 | 58141828 | 58141847 | +/− | 20 | CCCAACTAATCTCCATTTGC | 808 |
| chr3 | 58141829 | 58141848 | +/− | 20 | CCAACTAATCTCCATTTGCC | 809 |
| chr3 | 58141830 | 58141849 | +/− | 20 | CAACTAATCTCCATTTGCCA | 810 |
| chr3 | 58141831 | 58141850 | +/− | 20 | AACTAATCTCCATTTGCCAC | 811 |
| chr3 | 58141832 | 58141851 | +/− | 20 | ACTAATCTCCATTTGCCACT | 812 |
| chr3 | 58141833 | 58141852 | +/− | 20 | CTAATCTCCATTTGCCACTG | 813 |
| chr3 | 58141834 | 58141853 | +/− | 20 | TAATCTCCATTTGCCACTGA | 814 |
| chr3 | 58141835 | 58141854 | +/− | 20 | AATCTCCATTTGCCACTGAC | 815 |
| chr3 | 58141836 | 58141855 | +/− | 20 | ATCTCCATTTGCCACTGACC | 816 |
| chr3 | 58141837 | 58141856 | +/− | 20 | TCTCCATTTGCCACTGACCA | 817 |
| chr3 | 58141838 | 58141857 | +/− | 20 | CTCCATTTGCCACTGACCAG | 818 |
| chr3 | 58141839 | 58141858 | +/− | 20 | TCCATTTGCCACTGACCAGG | 819 |
| chr3 | 58141840 | 58141859 | +/− | 20 | CCATTTGCCACTGACCAGGC | 820 |
| chr3 | 58141841 | 58141860 | +/− | 20 | CATTTGCCACTGACCAGGCC | 821 |
| chr3 | 58141842 | 58141861 | +/− | 20 | ATTTGCCACTGACCAGGCCA | 822 |
| chr3 | 58141843 | 58141862 | +/− | 20 | TTTGCCACTGACCAGGCCAC | 823 |
| chr3 | 58141844 | 58141863 | +/− | 20 | TTGCCACTGACCAGGCCACA | 824 |
| chr3 | 58141845 | 58141864 | +/− | 20 | TGCCACTGACCAGGCCACAG | 825 |
| chr3 | 58141846 | 58141865 | +/− | 20 | GCCACTGACCAGGCCACAGA | 826 |
| chr3 | 58141847 | 58141866 | +/− | 20 | CCACTGACCAGGCCACAGAT | 827 |
| chr3 | 58141848 | 58141867 | +/− | 20 | CACTGACCAGGCCACAGATG | 828 |
| chr3 | 58141849 | 58141868 | +/− | 20 | ACTGACCAGGCCACAGATGG | 829 |
| chr3 | 58141850 | 58141869 | +/− | 20 | CTGACCAGGCCACAGATGGG | 830 |
| chr3 | 58141851 | 58141870 | +/− | 20 | TGACCAGGCCACAGATGGGG | 831 |
| chr3 | 58141852 | 58141871 | +/− | 20 | GACCAGGCCACAGATGGGGA | 832 |
| chr3 | 58141853 | 58141872 | +/− | 20 | ACCAGGCCACAGATGGGGAA | 833 |
| chr3 | 58141854 | 58141873 | +/− | 20 | CCAGGCCACAGATGGGGAAG | 834 |
| chr3 | 58141855 | 58141874 | +/− | 20 | CAGGCCACAGATGGGGAAGT | 835 |
| chr3 | 58141856 | 58141875 | +/− | 20 | AGGCCACAGATGGGGAAGTC | 836 |
| chr3 | 58141857 | 58141876 | +/− | 20 | GGCCACAGATGGGGAAGTCA | 837 |
| chr3 | 58141858 | 58141877 | +/− | 20 | GCCACAGATGGGGAAGTCAC | 838 |
| chr3 | 58141859 | 58141878 | +/− | 20 | CCACAGATGGGGAAGTCACA | 839 |
| chr3 | 58141860 | 58141879 | +/− | 20 | CACAGATGGGGAAGTCACAG | 840 |
| chr3 | 58141861 | 58141880 | +/− | 20 | ACAGATGGGGAAGTCACAGC | 841 |
| chr3 | 58141862 | 58141881 | +/− | 20 | CAGATGGGGAAGTCACAGCC | 842 |
| chr3 | 58141863 | 58141882 | +/− | 20 | AGATGGGGAAGTCACAGCCG | 843 |
| chr3 | 58141864 | 58141883 | +/− | 20 | GATGGGGAAGTCACAGCCGT | 844 |
| chr3 | 58141865 | 58141884 | +/− | 20 | ATGGGGAAGTCACAGCCGTG | 845 |
| chr3 | 58141866 | 58141885 | +/− | 20 | TGGGGAAGTCACAGCCGTGG | 846 |
| chr3 | 58141867 | 58141886 | +/− | 20 | GGGGAAGTCACAGCCGTGGA | 847 |
| chr3 | 58141868 | 58141887 | +/− | 20 | GGGAAGTCACAGCCGTGGAG | 848 |
| chr3 | 58141869 | 58141888 | +/− | 20 | GGAAGTCACAGCCGTGGAGG | 849 |
| chr3 | 58141870 | 58141889 | +/− | 20 | GAAGTCACAGCCGTGGAGGA | 850 |
| chr3 | 58141871 | 58141890 | +/− | 20 | AAGTCACAGCCGTGGAGGAG | 851 |
| chr3 | 58141872 | 58141891 | +/− | 20 | AGTCACAGCCGTGGAGGAGG | 852 |
| chr3 | 58141873 | 58141892 | +/− | 20 | GTCACAGCCGTGGAGGAGGC | 853 |
| chr3 | 58141874 | 58141893 | +/− | 20 | TCACAGCCGTGGAGGAGGCA | 854 |
| chr3 | 58141875 | 58141894 | +/− | 20 | CACAGCCGTGGAGGAGGCAC | 855 |
| chr3 | 58141876 | 58141895 | +/− | 20 | ACAGCCGTGGAGGAGGCACC | 856 |
| chr3 | 58141877 | 58141896 | +/− | 20 | CAGCCGTGGAGGAGGCACCG | 857 |
| chr3 | 58141878 | 58141897 | +/− | 20 | AGCCGTGGAGGAGGCACCGG | 858 |
| chr3 | 58141879 | 58141898 | +/− | 20 | GCCGTGGAGGAGGCACCGGT | 859 |
| chr3 | 58141880 | 58141899 | +/− | 20 | CCGTGGAGGAGGCACCGGTA | 860 |
| chr3 | 58141881 | 58141900 | +/− | 20 | CGTGGAGGAGGCACCGGTAA | 861 |
| chr3 | 58141882 | 58141901 | +/− | 20 | GTGGAGGAGGCACCGGTAAA | 862 |
| chr3 | 58141883 | 58141902 | +/− | 20 | TGGAGGAGGCACCGGTAAAT | 863 |
| chr3 | 58141884 | 58141903 | +/− | 20 | GGAGGAGGCACCGGTAAATG | 864 |
| chr3 | 58141885 | 58141904 | +/− | 20 | GAGGAGGCACCGGTAAATGC | 865 |
| chr3 | 58141886 | 58141905 | +/− | 20 | AGGAGGCACCGGTAAATGCA | 866 |
| chr3 | 58141887 | 58141906 | +/− | 20 | GGAGGCACCGGTAAATGCAT | 867 |
| chr3 | 58141888 | 58141907 | +/− | 20 | GAGGCACCGGTAAATGCATG | 868 |
| chr3 | 58141889 | 58141908 | +/− | 20 | AGGCACCGGTAAATGCATGT | 869 |
| chr3 | 58141890 | 58141909 | +/− | 20 | GGCACCGGTAAATGCATGTC | 870 |
| chr3 | 58141891 | 58141910 | +/− | 20 | GCACCGGTAAATGCATGTCC | 871 |
| chr3 | 58141892 | 58141911 | +/− | 20 | CACCGGTAAATGCATGTCCC | 872 |
| chr3 | 58141893 | 58141912 | +/− | 20 | ACCGGTAAATGCATGTCCCC | 873 |
| chr3 | 58141894 | 58141913 | +/− | 20 | CCGGTAAATGCATGTCCCCC | 874 |
| chr3 | 58141895 | 58141914 | +/− | 20 | CGGTAAATGCATGTCCCCCT | 875 |
| chr3 | 58141896 | 58141915 | +/− | 20 | GGTAAATGCATGTCCCCCTG | 876 |
| chr3 | 58141897 | 58141916 | +/− | 20 | GTAAATGCATGTCCCCCTGG | 877 |
| chr3 | 58141898 | 58141917 | +/− | 20 | TAAATGCATGTCCCCCTGGA | 878 |
| chr3 | 58141899 | 58141918 | +/− | 20 | AAATGCATGTCCCCCTGGAT | 879 |
| chr3 | 58141900 | 58141919 | +/− | 20 | AATGCATGTCCCCCTGGATT | 880 |
| chr3 | 58141901 | 58141920 | +/− | 20 | ATGCATGTCCCCCTGGATTC | 881 |
| chr3 | 58141902 | 58141921 | +/− | 20 | TGCATGTCCCCCTGGATTCA | 882 |
| chr3 | 58141903 | 58141922 | +/− | 20 | GCATGTCCCCCTGGATTCAG | 883 |
| chr3 | 58141904 | 58141923 | +/− | 20 | CATGTCCCCCTGGATTCAGG | 884 |
| chr3 | 58141905 | 58141924 | +/− | 20 | ATGTCCCCCTGGATTCAGGC | 885 |
| chr3 | 58141906 | 58141925 | +/− | 20 | TGTCCCCCTGGATTCAGGCC | 886 |
| chr3 | 58141907 | 58141926 | +/− | 20 | GTCCCCCTGGATTCAGGCCC | 887 |
| chr3 | 58141908 | 58141927 | +/− | 20 | TCCCCCTGGATTCAGGCCCT | 888 |
| chr3 | 58141909 | 58141928 | +/− | 20 | CCCCCTGGATTCAGGCCCTG | 889 |
| chr3 | 58141910 | 58141929 | +/− | 20 | CCCCTGGATTCAGGCCCTGG | 890 |
| chr3 | 58141911 | 58141930 | +/− | 20 | CCCTGGATTCAGGCCCTGGG | 891 |
| chr3 | 58141912 | 58141931 | +/− | 20 | CCTGGATTCAGGCCCTGGGT | 892 |
| chr3 | 58141913 | 58141932 | +/− | 20 | CTGGATTCAGGCCCTGGGTA | 893 |
| chr3 | 58141914 | 58141933 | +/− | 20 | TGGATTCAGGCCCTGGGTAC | 894 |
| chr3 | 58141915 | 58141934 | +/− | 20 | GGATTCAGGCCCTGGGTACA | 895 |
| chr3 | 58141916 | 58141935 | +/− | 20 | GATTCAGGCCCTGGGTACAA | 896 |
| chr3 | 58141917 | 58141936 | +/− | 20 | ATTCAGGCCCTGGGTACAAT | 897 |
| chr3 | 58141918 | 58141937 | +/− | 20 | TTCAGGCCCTGGGTACAATT | 898 |
| chr3 | 58141919 | 58141938 | +/− | 20 | TCAGGCCCTGGGTACAATTT | 899 |
| chr3 | 58141920 | 58141939 | +/− | 20 | CAGGCCCTGGGTACAATTTT | 900 |
| chr3 | 58141921 | 58141940 | +/− | 20 | AGGCCCTGGGTACAATTTTG | 901 |
| chr3 | 58141922 | 58141941 | +/− | 20 | GGCCCTGGGTACAATTTTGG | 902 |
| chr3 | 58141923 | 58141942 | +/− | 20 | GCCCTGGGTACAATTTTGGT | 903 |
| chr3 | 58141924 | 58141943 | +/− | 20 | CCCTGGGTACAATTTTGGTT | 904 |
| chr3 | 58141925 | 58141944 | +/− | 20 | CCTGGGTACAATTTTGGTTT | 905 |
| chr3 | 58141926 | 58141945 | +/− | 20 | CTGGGTACAATTTTGGTTTT | 906 |
| chr3 | 58141927 | 58141946 | +/− | 20 | TGGGTACAATTTTGGTTTTT | 907 |
| chr3 | 58141928 | 58141947 | +/− | 20 | GGGTACAATTTTGGTTTTTT | 908 |
| chr3 | 58141929 | 58141948 | +/− | 20 | GGTACAATTTTGGTTTTTTC | 909 |
| chr3 | 58141930 | 58141949 | +/− | 20 | GTACAATTTTGGTTTTTTCC | 910 |
| chr3 | 58141931 | 58141950 | +/− | 20 | TACAATTTTGGTTTTTTCCT | 911 |
| chr3 | 58141932 | 58141951 | +/− | 20 | ACAATTTTGGTTTTTTCCTT | 912 |
| chr3 | 58141933 | 58141952 | +/− | 20 | CAATTTTGGTTTTTTCCTTT | 913 |
| chr3 | 58141934 | 58141953 | +/− | 20 | AATTTTGGTTTTTTCCTTTT | 914 |
| chr3 | 58141935 | 58141954 | +/− | 20 | ATTTTGGTTTTTTCCTTTTT | 915 |
| chr3 | 58141936 | 58141955 | +/− | 20 | TTTTGGTTTTTTCCTTTTTG | 916 |
| chr3 | 58141937 | 58141956 | +/− | 20 | TTTGGTTTTTTCCTTTTTGT | 917 |
| chr3 | 58141938 | 58141957 | +/− | 20 | TTGGTTTTTTCCTTTTTGTG | 918 |
| chr3 | 58141939 | 58141958 | +/− | 20 | TGGTTTTTTCCTTTTTGTGT | 919 |
| chr3 | 58141940 | 58141959 | +/− | 20 | GGTTTTTTCCTTTTTGTGTT | 920 |
| chr3 | 58141941 | 58141960 | +/− | 20 | GTTTTTTCCTTTTTGTGTTT | 921 |
| chr3 | 58141942 | 58141961 | +/− | 20 | TTTTTTCCTTTTTGTGTTTC | 922 |
| chr3 | 58141943 | 58141962 | +/− | 20 | TTTTTCCTTTTTGTGTTTCT | 923 |
| chr3 | 58141944 | 58141963 | +/− | 20 | TTTTCCTTTTTGTGTTTCTG | 924 |
| chr3 | 58141945 | 58141964 | +/− | 20 | TTTCCTTTTTGTGTTTCTGT | 925 |
| chr3 | 58141946 | 58141965 | +/− | 20 | TTCCTTTTTGTGTTTCTGTG | 926 |
| chr3 | 58141947 | 58141966 | +/− | 20 | TCCTTTTTGTGTTTCTGTGT | 927 |
| chr3 | 58141948 | 58141967 | +/− | 20 | CCTTTTTGTGTTTCTGTGTT | 928 |
| chr3 | 58141949 | 58141968 | +/− | 20 | CTTTTTGTGTTTCTGTGTTT | 929 |
| chr3 | 58141950 | 58141969 | +/− | 20 | TTTTTGTGTTTCTGTGTTTA | 930 |
| chr3 | 58141951 | 58141970 | +/− | 20 | TTTTGTGTTTCTGTGTTTAC | 931 |
| chr3 | 58141952 | 58141971 | +/− | 20 | TTTGTGTTTCTGTGTTTACT | 932 |
| chr3 | 58141953 | 58141972 | +/− | 20 | TTGTGTTTCTGTGTTTACTC | 933 |
| chr3 | 58141954 | 58141973 | +/− | 20 | TGTGTTTCTGTGTTTACTCA | 934 |
| chr3 | 58141955 | 58141974 | +/− | 20 | GTGTTTCTGTGTTTACTCAG | 935 |
| chr3 | 58141956 | 58141975 | +/− | 20 | TGTTTCTGTGTTTACTCAGC | 936 |
| chr3 | 58141957 | 58141976 | +/− | 20 | GTTTCTGTGTTTACTCAGCC | 937 |
| chr3 | 58141958 | 58141977 | +/− | 20 | TTTCTGTGTTTACTCAGCCT | 938 |
| chr3 | 58141959 | 58141978 | +/− | 20 | TTCTGTGTTTACTCAGCCTT | 939 |
| chr3 | 58141960 | 58141979 | +/− | 20 | TCTGTGTTTACTCAGCCTTC | 940 |
| chr3 | 58141961 | 58141980 | +/− | 20 | CTGTGTTTACTCAGCCTTCA | 941 |
| chr3 | 58141962 | 58141981 | +/− | 20 | TGTGTTTACTCAGCCTTCAT | 942 |
| chr3 | 58141963 | 58141982 | +/− | 20 | GTGTTTACTCAGCCTTCATT | 943 |
| chr3 | 58141964 | 58141983 | +/− | 20 | TGTTTACTCAGCCTTCATTT | 944 |
| chr3 | 58141965 | 58141984 | +/− | 20 | GTTTACTCAGCCTTCATTTC | 945 |
| chr3 | 58141966 | 58141985 | +/− | 20 | TTTACTCAGCCTTCATTTCA | 946 |
| chr3 | 58141967 | 58141986 | +/− | 20 | TTACTCAGCCTTCATTTCAG | 947 |
| chr3 | 58141968 | 58141987 | +/− | 20 | TACTCAGCCTTCATTTCAGA | 948 |
| chr3 | 58141969 | 58141988 | +/− | 20 | ACTCAGCCTTCATTTCAGAA | 949 |
| chr3 | 58141970 | 58141989 | +/− | 20 | CTCAGCCTTCATTTCAGAAA | 950 |
| chr3 | 58141971 | 58141990 | +/− | 20 | TCAGCCTTCATTTCAGAAAA | 951 |
| chr3 | 58141972 | 58141991 | +/− | 20 | CAGCCTTCATTTCAGAAAAT | 952 |
| chr3 | 58141973 | 58141992 | +/− | 20 | AGCCTTCATTTCAGAAAATC | 953 |
| chr3 | 58141974 | 58141993 | +/− | 20 | GCCTTCATTTCAGAAAATCT | 954 |
| chr3 | 58141975 | 58141994 | +/− | 20 | CCTTCATTTCAGAAAATCTG | 955 |
| chr3 | 58141976 | 58141995 | +/− | 20 | CTTCATTTCAGAAAATCTGC | 956 |
| chr3 | 58141977 | 58141996 | +/− | 20 | TTCATTTCAGAAAATCTGCC | 957 |
| chr3 | 58141978 | 58141997 | +/− | 20 | TCATTTCAGAAAATCTGCCA | 958 |
| chr3 | 58141979 | 58141998 | +/− | 20 | CATTTCAGAAAATCTGCCAT | 959 |
| chr3 | 58141980 | 58141999 | +/− | 20 | ATTTCAGAAAATCTGCCATC | 960 |
| chr3 | 58141981 | 58142000 | +/− | 20 | TTTCAGAAAATCTGCCATCT | 961 |
| chr3 | 58141982 | 58142001 | +/− | 20 | TTCAGAAAATCTGCCATCTG | 962 |
| chr3 | 58141983 | 58142002 | +/− | 20 | TCAGAAAATCTGCCATCTGC | 963 |
| chr3 | 58141984 | 58142003 | +/− | 20 | CAGAAAATCTGCCATCTGCT | 964 |
| chr3 | 58141985 | 58142004 | +/− | 20 | AGAAAATCTGCCATCTGCTT | 965 |
| chr3 | 58141986 | 58142005 | +/− | 20 | GAAAATCTGCCATCTGCTTC | 966 |
| chr3 | 58141987 | 58142006 | +/− | 20 | AAAATCTGCCATCTGCTTCT | 967 |
| chr3 | 58141988 | 58142007 | +/− | 20 | AAATCTGCCATCTGCTTCTG | 968 |
| chr3 | 58141989 | 58142008 | +/− | 20 | AATCTGCCATCTGCTTCTGG | 969 |
| chr3 | 58141990 | 58142009 | +/− | 20 | ATCTGCCATCTGCTTCTGGG | 970 |
| chr3 | 58141991 | 58142010 | +/− | 20 | TCTGCCATCTGCTTCTGGGA | 971 |
| chr3 | 58141992 | 58142011 | +/− | 20 | CTGCCATCTGCTTCTGGGAT | 972 |
| chr3 | 58141993 | 58142012 | +/− | 20 | TGCCATCTGCTTCTGGGATT | 973 |
| chr3 | 58141994 | 58142013 | +/− | 20 | GCCATCTGCTTCTGGGATTG | 974 |
| chr3 | 58141995 | 58142014 | +/− | 20 | CCATCTGCTTCTGGGATTGC | 975 |
| chr3 | 58141996 | 58142015 | +/− | 20 | CATCTGCTTCTGGGATTGCT | 976 |
| chr3 | 58141997 | 58142016 | +/− | 20 | ATCTGCTTCTGGGATTGCTT | 977 |
| chr3 | 58141998 | 58142017 | +/− | 20 | TCTGCTTCTGGGATTGCTTA | 978 |
| chr3 | 58141999 | 58142018 | +/− | 20 | CTGCTTCTGGGATTGCTTAA | 979 |
| chr3 | 58142000 | 58142019 | +/− | 20 | TGCTTCTGGGATTGCTTAAG | 980 |
| chr3 | 58142001 | 58142020 | +/− | 20 | GCTTCTGGGATTGCTTAAGC | 981 |
| chr3 | 58142002 | 58142021 | +/− | 20 | CTTCTGGGATTGCTTAAGCC | 982 |
| chr3 | 58142003 | 58142022 | +/− | 20 | TTCTGGGATTGCTTAAGCCC | 983 |
| chr3 | 58142004 | 58142023 | +/− | 20 | TCTGGGATTGCTTAAGCCCT | 984 |
| chr3 | 58142005 | 58142024 | +/− | 20 | CTGGGATTGCTTAAGCCCTG | 985 |
| chr3 | 58142006 | 58142025 | +/− | 20 | TGGGATTGCTTAAGCCCTGT | 986 |
| chr3 | 58142007 | 58142026 | +/− | 20 | GGGATTGCTTAAGCCCTGTG | 987 |
| chr3 | 58142008 | 58142027 | +/− | 20 | GGATTGCTTAAGCCCTGTGG | 988 |
| chr3 | 58142009 | 58142028 | +/− | 20 | GATTGCTTAAGCCCTGTGGG | 989 |
| chr3 | 58142010 | 58142029 | +/− | 20 | ATTGCTTAAGCCCTGTGGGT | 990 |
| chr3 | 58142011 | 58142030 | +/− | 20 | TTGCTTAAGCCCTGTGGGTG | 991 |
| chr3 | 58142012 | 58142031 | +/− | 20 | TGCTTAAGCCCTGTGGGTGT | 992 |
| chr3 | 58142013 | 58142032 | +/− | 20 | GCTTAAGCCCTGTGGGTGTC | 993 |
| chr3 | 58142014 | 58142033 | +/− | 20 | CTTAAGCCCTGTGGGTGTCC | 994 |
| chr3 | 58142015 | 58142034 | +/− | 20 | TTAAGCCCTGTGGGTGTCCT | 995 |
| chr3 | 58142016 | 58142035 | +/− | 20 | TAAGCCCTGTGGGTGTCCTG | 996 |
| chr3 | 58142017 | 58142036 | +/− | 20 | AAGCCCTGTGGGTGTCCTGG | 997 |
| chr3 | 58142018 | 58142037 | +/− | 20 | AGCCCTGTGGGTGTCCTGGT | 998 |
| chr3 | 58142019 | 58142038 | +/− | 20 | GCCCTGTGGGTGTCCTGGTC | 999 |
| chr3 | 58142020 | 58142039 | +/− | 20 | CCCTGTGGGTGTCCTGGTCA | 1000 |
| chr3 | 58142021 | 58142040 | +/− | 20 | CCTGTGGGTGTCCTGGTCAT | 1001 |
| chr3 | 58142022 | 58142041 | +/− | 20 | CTGTGGGTGTCCTGGTCATT | 1002 |
| chr3 | 58142023 | 58142042 | +/− | 20 | TGTGGGTGTCCTGGTCATTG | 1003 |
| chr3 | 58142024 | 58142043 | +/− | 20 | GTGGGTGTCCTGGTCATTGG | 1004 |
| chr3 | 58142025 | 58142044 | +/− | 20 | TGGGTGTCCTGGTCATTGGT | 1005 |

TABLE 7
21-mer target-specific ASOs

| | | | | | | SEQ |
|---|---|---|---|---|---|---|
| | | | | | | ID |
| CHR | START | END | STRAND | kmer | SEQUENCE | NO: |
| chr3 | 58141828 | 58141848 | +/− | 21 | CCCAACTAATCTCCATTTGCC | 1006 |
| chr3 | 58141829 | 58141849 | +/− | 21 | CCAACTAATCTCCATTTGCCA | 1007 |
| chr3 | 58141830 | 58141850 | +/− | 21 | CAACTAATCTCCATTTGCCAC | 1008 |
| chr3 | 58141831 | 58141851 | +/− | 21 | AACTAATCTCCATTTGCCACT | 1009 |
| chr3 | 58141832 | 58141852 | +/− | 21 | ACTAATCTCCATTTGCCACTG | 1010 |
| chr3 | 58141833 | 58141853 | +/− | 21 | CTAATCTCCATTTGCCACTGA | 1011 |
| chr3 | 58141834 | 58141854 | +/− | 21 | TAATCTCCATTTGCCACTGAC | 1012 |
| chr3 | 58141835 | 58141855 | +/− | 21 | AATCTCCATTTGCCACTGACC | 1013 |
| chr3 | 58141836 | 58141856 | +/− | 21 | ATCTCCATTTGCCACTGACCA | 1014 |
| chr3 | 58141837 | 58141857 | +/− | 21 | TCTCCATTTGCCACTGACCAG | 1015 |
| chr3 | 58141838 | 58141858 | +/− | 21 | CTCCATTTGCCACTGACCAGG | 1016 |
| chr3 | 58141839 | 58141859 | +/− | 21 | TCCATTTGCCACTGACCAGGC | 1017 |
| chr3 | 58141840 | 58141860 | +/− | 21 | CCATTTGCCACTGACCAGGCC | 1018 |
| chr3 | 58141841 | 58141861 | +/− | 21 | CATTTGCCACTGACCAGGCCA | 1019 |
| chr3 | 58141842 | 58141862 | +/− | 21 | ATTTGCCACTGACCAGGCCAC | 1020 |
| chr3 | 58141843 | 58141863 | +/− | 21 | TTTGCCACTGACCAGGCCACA | 1021 |
| chr3 | 58141844 | 58141864 | +/− | 21 | TTGCCACTGACCAGGCCACAG | 1022 |
| chr3 | 58141845 | 58141865 | +/− | 21 | TGCCACTGACCAGGCCACAGA | 1023 |
| chr3 | 58141846 | 58141866 | +/− | 21 | GCCACTGACCAGGCCACAGAT | 1024 |
| chr3 | 58141847 | 58141867 | +/− | 21 | CCACTGACCAGGCCACAGATG | 1025 |
| chr3 | 58141848 | 58141868 | +/− | 21 | CACTGACCAGGCCACAGATGG | 1026 |
| chr3 | 58141849 | 58141869 | +/− | 21 | ACTGACCAGGCCACAGATGGG | 1027 |
| chr3 | 58141850 | 58141870 | +/− | 21 | CTGACCAGGCCACAGATGGGG | 1028 |
| chr3 | 58141851 | 58141871 | +/− | 21 | TGACCAGGCCACAGATGGGGA | 1029 |
| chr3 | 58141852 | 58141872 | +/− | 21 | GACCAGGCCACAGATGGGGAA | 1030 |
| chr3 | 58141853 | 58141873 | +/− | 21 | ACCAGGCCACAGATGGGGAAG | 1031 |
| chr3 | 58141854 | 58141874 | +/− | 21 | CCAGGCCACAGATGGGGAAGT | 1032 |
| chr3 | 58141855 | 58141875 | +/− | 21 | CAGGCCACAGATGGGGAAGTC | 1033 |
| chr3 | 58141856 | 58141876 | +/− | 21 | AGGCCACAGATGGGGAAGTCA | 1034 |
| chr3 | 58141857 | 58141877 | +/− | 21 | GGCCACAGATGGGGAAGTCAC | 1035 |
| chr3 | 58141858 | 58141878 | +/− | 21 | GCCACAGATGGGGAAGTCACA | 1036 |
| chr3 | 58141859 | 58141879 | +/− | 21 | CCACAGATGGGGAAGTCACAG | 1037 |
| chr3 | 58141860 | 58141880 | +/− | 21 | CACAGATGGGGAAGTCACAGC | 1038 |
| chr3 | 58141861 | 58141881 | +/− | 21 | ACAGATGGGGAAGTCACAGCC | 1039 |
| chr3 | 58141862 | 58141882 | +/− | 21 | CAGATGGGGAAGTCACAGCCG | 1040 |
| chr3 | 58141863 | 58141883 | +/− | 21 | AGATGGGGAAGTCACAGCCGT | 1041 |
| chr3 | 58141864 | 58141884 | +/− | 21 | GATGGGGAAGTCACAGCCGTG | 1042 |
| chr3 | 58141865 | 58141885 | +/− | 21 | ATGGGGAAGTCACAGCCGTGG | 1043 |
| chr3 | 58141866 | 58141886 | +/− | 21 | TGGGGAAGTCACAGCCGTGGA | 1044 |
| chr3 | 58141867 | 58141887 | +/− | 21 | GGGGAAGTCACAGCCGTGGAG | 1045 |
| chr3 | 58141868 | 58141888 | +/− | 21 | GGGAAGTCACAGCCGTGGAGG | 1046 |
| chr3 | 58141869 | 58141889 | +/− | 21 | GGAAGTCACAGCCGTGGAGGA | 1047 |
| chr3 | 58141870 | 58141890 | +/− | 21 | GAAGTCACAGCCGTGGAGGAG | 1048 |
| chr3 | 58141871 | 58141891 | +/− | 21 | AAGTCACAGCCGTGGAGGAGG | 1049 |
| chr3 | 58141872 | 58141892 | +/− | 21 | AGTCACAGCCGTGGAGGAGGC | 1050 |
| chr3 | 58141873 | 58141893 | +/− | 21 | GTCACAGCCGTGGAGGAGGCA | 1051 |
| chr3 | 58141874 | 58141894 | +/− | 21 | TCACAGCCGTGGAGGAGGCAC | 1052 |
| chr3 | 58141875 | 58141895 | +/− | 21 | CACAGCCGTGGAGGAGGCACC | 1053 |
| chr3 | 58141876 | 58141896 | +/− | 21 | ACAGCCGTGGAGGAGGCACCG | 1054 |
| chr3 | 58141877 | 58141897 | +/− | 21 | CAGCCGTGGAGGAGGCACCGG | 1055 |
| chr3 | 58141878 | 58141898 | +/− | 21 | AGCCGTGGAGGAGGCACCGGT | 1056 |
| chr3 | 58141879 | 58141899 | +/− | 21 | GCCGTGGAGGAGGCACCGGTA | 1057 |
| chr3 | 58141880 | 58141900 | +/− | 21 | CCGTGGAGGAGGCACCGGTAA | 1058 |
| chr3 | 58141881 | 58141901 | +/− | 21 | CGTGGAGGAGGCACCGGTAAA | 1059 |
| chr3 | 58141882 | 58141902 | +/− | 21 | GTGGAGGAGGCACCGGTAAAT | 1060 |
| chr3 | 58141883 | 58141903 | +/− | 21 | TGGAGGAGGCACCGGTAAATG | 1061 |
| chr3 | 58141884 | 58141904 | +/− | 21 | GGAGGAGGCACCGGTAAATGC | 1062 |
| chr3 | 58141885 | 58141905 | +/− | 21 | GAGGAGGCACCGGTAAATGCA | 1063 |
| chr3 | 58141886 | 58141906 | +/− | 21 | AGGAGGCACCGGTAAATGCAT | 1064 |
| chr3 | 58141887 | 58141907 | +/− | 21 | GGAGGCACCGGTAAATGCATG | 1065 |
| chr3 | 58141888 | 58141908 | +/− | 21 | GAGGCACCGGTAAATGCATGT | 1066 |
| chr3 | 58141889 | 58141909 | +/− | 21 | AGGCACCGGTAAATGCATGTC | 1067 |
| chr3 | 58141890 | 58141910 | +/− | 21 | GGCACCGGTAAATGCATGTCC | 1068 |
| chr3 | 58141891 | 58141911 | +/− | 21 | GCACCGGTAAATGCATGTCCC | 1069 |
| chr3 | 58141892 | 58141912 | +/− | 21 | CACCGGTAAATGCATGTCCCC | 1070 |
| chr3 | 58141893 | 58141913 | +/− | 21 | ACCGGTAAATGCATGTCCCCC | 1071 |
| chr3 | 58141894 | 58141914 | +/− | 21 | CCGGTAAATGCATGTCCCCCT | 1072 |
| chr3 | 58141895 | 58141915 | +/− | 21 | CGGTAAATGCATGTCCCCCTG | 1073 |
| chr3 | 58141896 | 58141916 | +/− | 21 | GGTAAATGCATGTCCCCCTGG | 1074 |
| chr3 | 58141897 | 58141917 | +/− | 21 | GTAAATGCATGTCCCCCTGGA | 1075 |
| chr3 | 58141898 | 58141918 | +/− | 21 | TAAATGCATGTCCCCCTGGAT | 1076 |
| chr3 | 58141899 | 58141919 | +/− | 21 | AAATGCATGTCCCCCTGGATT | 1077 |
| chr3 | 58141900 | 58141920 | +/− | 21 | AATGCATGTCCCCCTGGATTC | 1078 |
| chr3 | 58141901 | 58141921 | +/− | 21 | ATGCATGTCCCCCTGGATTCA | 1079 |
| chr3 | 58141902 | 58141922 | +/− | 21 | TGCATGTCCCCCTGGATTCAG | 1080 |
| chr3 | 58141903 | 58141923 | +/− | 21 | GCATGTCCCCCTGGATTCAGG | 1081 |
| chr3 | 58141904 | 58141924 | +/− | 21 | CATGTCCCCCTGGATTCAGGC | 1082 |
| chr3 | 58141905 | 58141925 | +/− | 21 | ATGTCCCCCTGGATTCAGGCC | 1083 |
| chr3 | 58141906 | 58141926 | +/− | 21 | TGTCCCCCTGGATTCAGGCCC | 1084 |
| chr3 | 58141907 | 58141927 | +/− | 21 | GTCCCCCTGGATTCAGGCCCT | 1085 |
| chr3 | 58141908 | 58141928 | +/− | 21 | TCCCCCTGGATTCAGGCCCTG | 1086 |
| chr3 | 58141909 | 58141929 | +/− | 21 | CCCCCTGGATTCAGGCCCTGG | 1087 |
| chr3 | 58141910 | 58141930 | +/− | 21 | CCCCTGGATTCAGGCCCTGGG | 1088 |
| chr3 | 58141911 | 58141931 | +/− | 21 | CCCTGGATTCAGGCCCTGGGT | 1089 |
| chr3 | 58141912 | 58141932 | +/− | 21 | CCTGGATTCAGGCCCTGGGTA | 1090 |
| chr3 | 58141913 | 58141933 | +/− | 21 | CTGGATTCAGGCCCTGGGTAC | 1091 |
| chr3 | 58141914 | 58141934 | +/− | 21 | TGGATTCAGGCCCTGGGTACA | 1092 |
| chr3 | 58141915 | 58141935 | +/− | 21 | GGATTCAGGCCCTGGGTACAA | 1093 |
| chr3 | 58141916 | 58141936 | +/− | 21 | GATTCAGGCCCTGGGTACAAT | 1094 |
| chr3 | 58141917 | 58141937 | +/− | 21 | ATTCAGGCCCTGGGTACAATT | 1095 |
| chr3 | 58141918 | 58141938 | +/− | 21 | TTCAGGCCCTGGGTACAATTT | 1096 |
| chr3 | 58141919 | 58141939 | +/− | 21 | TCAGGCCCTGGGTACAATTTT | 1097 |
| chr3 | 58141920 | 58141940 | +/− | 21 | CAGGCCCTGGGTACAATTTTG | 1098 |
| chr3 | 58141921 | 58141941 | +/− | 21 | AGGCCCTGGGTACAATTTTGG | 1099 |
| chr3 | 58141922 | 58141942 | +/− | 21 | GGCCCTGGGTACAATTTTGGT | 1100 |
| chr3 | 58141923 | 58141943 | +/− | 21 | GCCCTGGGTACAATTTTGGTT | 1101 |
| chr3 | 58141924 | 58141944 | +/− | 21 | CCCTGGGTACAATTTTGGTTT | 1102 |
| chr3 | 58141925 | 58141945 | +/− | 21 | CCTGGGTACAATTTTGGTTTT | 1103 |
| chr3 | 58141926 | 58141946 | +/− | 21 | CTGGGTACAATTTTGGTTTTT | 1104 |
| chr3 | 58141927 | 58141947 | +/− | 21 | TGGGTACAATTTTGGTTTTTT | 1105 |
| chr3 | 58141928 | 58141948 | +/− | 21 | GGGTACAATTTTGGTTTTTTC | 1106 |
| chr3 | 58141929 | 58141949 | +/− | 21 | GGTACAATTTTGGTTTTTTCC | 1107 |
| chr3 | 58141930 | 58141950 | +/− | 21 | GTACAATTTTGGTTTTTTCCT | 1108 |
| chr3 | 58141931 | 58141951 | +/− | 21 | TACAATTTTGGTTTTTTCCTT | 1109 |
| chr3 | 58141932 | 58141952 | +/− | 21 | ACAATTTTGGTTTTTTCCTTT | 1110 |
| chr3 | 58141933 | 58141953 | +/− | 21 | CAATTTTGGTTTTTTCCTTTT | 1111 |
| chr3 | 58141934 | 58141954 | +/− | 21 | AATTTTGGTTTTTTCCTTTTT | 1112 |
| chr3 | 58141935 | 58141955 | +/− | 21 | ATTTTGGTTTTTTCCTTTTTG | 1113 |
| chr3 | 58141936 | 58141956 | +/− | 21 | TTTTGGTTTTTTCCTTTTTGT | 1114 |
| chr3 | 58141937 | 58141957 | +/− | 21 | TTTGGTTTTTTCCTTTTTGTG | 1115 |
| chr3 | 58141938 | 58141958 | +/− | 21 | TTGGTTTTTTCCTTTTTGTGT | 1116 |
| chr3 | 58141939 | 58141959 | +/− | 21 | TGGTTTTTTCCTTTTTGTGTT | 1117 |
| chr3 | 58141940 | 58141960 | +/− | 21 | GGTTTTTTCCTTTTTGTGTTT | 1118 |
| chr3 | 58141941 | 58141961 | +/− | 21 | GTTTTTTCCTTTTTGTGTTTC | 1119 |
| chr3 | 58141942 | 58141962 | +/− | 21 | TTTTTTCCTTTTTGTGTTTCT | 1120 |
| chr3 | 58141943 | 58141963 | +/− | 21 | TTTTTCCTTTTTGTGTTTCTG | 1121 |
| chr3 | 58141944 | 58141964 | +/− | 21 | TTTTCCTTTTTGTGTTTCTGT | 1122 |
| chr3 | 58141945 | 58141965 | +/− | 21 | TTTCCTTTTTGTGTTTCTGTG | 1123 |
| chr3 | 58141946 | 58141966 | +/− | 21 | TTCCTTTTTGTGTTTCTGTGT | 1124 |
| chr3 | 58141947 | 58141967 | +/− | 21 | TCCTTTTTGTGTTTCTGTGTT | 1125 |
| chr3 | 58141948 | 58141968 | +/− | 21 | CCTTTTTGTGTTTCTGTGTTT | 1126 |
| chr3 | 58141949 | 58141969 | +/− | 21 | CTTTTTGTGTTTCTGTGTTTA | 1127 |
| chr3 | 58141950 | 58141970 | +/− | 21 | TTTTTGTGTTTCTGTGTTTAC | 1128 |
| chr3 | 58141951 | 58141971 | +/− | 21 | TTTTGTGTTTCTGTGTTTACT | 1129 |
| chr3 | 58141952 | 58141972 | +/− | 21 | TTTGTGTTTCTGTGTTTACTC | 1130 |
| chr3 | 58141953 | 58141973 | +/− | 21 | TTGTGTTTCTGTGTTTACTCA | 1131 |
| chr3 | 58141954 | 58141974 | +/− | 21 | TGTGTTTCTGTGTTTACTCAG | 1132 |
| chr3 | 58141955 | 58141975 | +/− | 21 | GTGTTTCTGTGTTTACTCAGC | 1133 |
| chr3 | 58141956 | 58141976 | +/− | 21 | TGTTTCTGTGTTTACTCAGCC | 1134 |
| chr3 | 58141957 | 58141977 | +/− | 21 | GTTTCTGTGTTTACTCAGCCT | 1135 |
| chr3 | 58141958 | 58141978 | +/− | 21 | TTTCTGTGTTTACTCAGCCTT | 1136 |
| chr3 | 58141959 | 58141979 | +/− | 21 | TTCTGTGTTTACTCAGCCTTC | 1137 |
| chr3 | 58141960 | 58141980 | +/− | 21 | TCTGTGTTTACTCAGCCTTCA | 1138 |
| chr3 | 58141961 | 58141981 | +/− | 21 | CTGTGTTTACTCAGCCTTCAT | 1139 |
| chr3 | 58141962 | 58141982 | +/− | 21 | TGTGTTTACTCAGCCTTCATT | 1140 |
| chr3 | 58141963 | 58141983 | +/− | 21 | GTGTTTACTCAGCCTTCATTT | 1141 |
| chr3 | 58141964 | 58141984 | +/− | 21 | TGTTTACTCAGCCTTCATTTC | 1142 |
| chr3 | 58141965 | 58141985 | +/− | 21 | GTTTACTCAGCCTTCATTTCA | 1143 |
| chr3 | 58141966 | 58141986 | +/− | 21 | TTTACTCAGCCTTCATTTCAG | 1144 |
| chr3 | 58141967 | 58141987 | +/− | 21 | TTACTCAGCCTTCATTTCAGA | 1145 |
| chr3 | 58141968 | 58141988 | +/− | 21 | TACTCAGCCTTCATTTCAGAA | 1146 |
| chr3 | 58141969 | 58141989 | +/− | 21 | ACTCAGCCTTCATTTCAGAAA | 1147 |
| chr3 | 58141970 | 58141990 | +/− | 21 | CTCAGCCTTCATTTCAGAAAA | 1148 |
| chr3 | 58141971 | 58141991 | +/− | 21 | TCAGCCTTCATTTCAGAAAAT | 1149 |
| chr3 | 58141972 | 58141992 | +/− | 21 | CAGCCTTCATTTCAGAAAATC | 1150 |
| chr3 | 58141973 | 58141993 | +/− | 21 | AGCCTTCATTTCAGAAAATCT | 1151 |
| chr3 | 58141974 | 58141994 | +/− | 21 | GCCTTCATTTCAGAAAATCTG | 1152 |
| chr3 | 58141975 | 58141995 | +/− | 21 | CCTTCATTTCAGAAAATCTGC | 1153 |
| chr3 | 58141976 | 58141996 | +/− | 21 | CTTCATTTCAGAAAATCTGCC | 1154 |
| chr3 | 58141977 | 58141997 | +/− | 21 | TTCATTTCAGAAAATCTGCCA | 1155 |
| chr3 | 58141978 | 58141998 | +/− | 21 | TCATTTCAGAAAATCTGCCAT | 1156 |
| chr3 | 58141979 | 58141999 | +/− | 21 | CATTTCAGAAAATCTGCCATC | 1157 |
| chr3 | 58141980 | 58142000 | +/− | 21 | ATTTCAGAAAATCTGCCATCT | 1158 |
| chr3 | 58141981 | 58142001 | +/− | 21 | TTTCAGAAAATCTGCCATCTG | 1159 |
| chr3 | 58141982 | 58142002 | +/− | 21 | TTCAGAAAATCTGCCATCTGC | 1160 |
| chr3 | 58141983 | 58142003 | +/− | 21 | TCAGAAAATCTGCCATCTGCT | 1161 |
| chr3 | 58141984 | 58142004 | +/− | 21 | CAGAAAATCTGCCATCTGCTT | 1162 |
| chr3 | 58141985 | 58142005 | +/− | 21 | AGAAAATCTGCCATCTGCTTC | 1163 |
| chr3 | 58141986 | 58142006 | +/− | 21 | GAAAATCTGCCATCTGCTTCT | 1164 |
| chr3 | 58141987 | 58142007 | +/− | 21 | AAAATCTGCCATCTGCTTCTG | 1165 |
| chr3 | 58141988 | 58142008 | +/− | 21 | AAATCTGCCATCTGCTTCTGG | 1166 |
| chr3 | 58141989 | 58142009 | +/− | 21 | AATCTGCCATCTGCTTCTGGG | 1167 |
| chr3 | 58141990 | 58142010 | +/− | 21 | ATCTGCCATCTGCTTCTGGGA | 1168 |
| chr3 | 58141991 | 58142011 | +/− | 21 | TCTGCCATCTGCTTCTGGGAT | 1169 |
| chr3 | 58141992 | 58142012 | +/− | 21 | CTGCCATCTGCTTCTGGGATT | 1170 |
| chr3 | 58141993 | 58142013 | +/− | 21 | TGCCATCTGCTTCTGGGATTG | 1171 |
| chr3 | 58141994 | 58142014 | +/− | 21 | GCCATCTGCTTCTGGGATTGC | 1172 |
| chr3 | 58141995 | 58142015 | +/− | 21 | CCATCTGCTTCTGGGATTGCT | 1173 |
| chr3 | 58141996 | 58142016 | +/− | 21 | CATCTGCTTCTGGGATTGCTT | 1174 |
| chr3 | 58141997 | 58142017 | +/− | 21 | ATCTGCTTCTGGGATTGCTTA | 1175 |
| chr3 | 58141998 | 58142018 | +/− | 21 | TCTGCTTCTGGGATTGCTTAA | 1176 |
| chr3 | 58141999 | 58142019 | +/− | 21 | CTGCTTCTGGGATTGCTTAAG | 1177 |
| chr3 | 58142000 | 58142020 | +/− | 21 | TGCTTCTGGGATTGCTTAAGC | 1178 |
| chr3 | 58142001 | 58142021 | +/− | 21 | GCTTCTGGGATTGCTTAAGCC | 1179 |
| chr3 | 58142002 | 58142022 | +/− | 21 | CTTCTGGGATTGCTTAAGCCC | 1180 |
| chr3 | 58142003 | 58142023 | +/− | 21 | TTCTGGGATTGCTTAAGCCCT | 1181 |
| chr3 | 58142004 | 58142024 | +/− | 21 | TCTGGGATTGCTTAAGCCCTG | 1182 |
| chr3 | 58142005 | 58142025 | +/− | 21 | CTGGGATTGCTTAAGCCCTGT | 1183 |
| chr3 | 58142006 | 58142026 | +/− | 21 | TGGGATTGCTTAAGCCCTGTG | 1184 |
| chr3 | 58142007 | 58142027 | +/− | 21 | GGGATTGCTTAAGCCCTGTGG | 1185 |
| chr3 | 58142008 | 58142028 | +/− | 21 | GGATTGCTTAAGCCCTGTGGG | 1186 |
| chr3 | 58142009 | 58142029 | +/− | 21 | GATTGCTTAAGCCCTGTGGGT | 1187 |
| chr3 | 58142010 | 58142030 | +/− | 21 | ATTGCTTAAGCCCTGTGGGTG | 1188 |
| chr3 | 58142011 | 58142031 | +/− | 21 | TTGCTTAAGCCCTGTGGGTGT | 1189 |
| chr3 | 58142012 | 58142032 | +/− | 21 | TGCTTAAGCCCTGTGGGTGTC | 1190 |
| chr3 | 58142013 | 58142033 | +/− | 21 | GCTTAAGCCCTGTGGGTGTCC | 1191 |
| chr3 | 58142014 | 58142034 | +/− | 21 | CTTAAGCCCTGTGGGTGTCCT | 1192 |
| chr3 | 58142015 | 58142035 | +/− | 21 | TTAAGCCCTGTGGGTGTCCTG | 1193 |
| chr3 | 58142016 | 58142036 | +/− | 21 | TAAGCCCTGTGGGTGTCCTGG | 1194 |
| chr3 | 58142017 | 58142037 | +/− | 21 | AAGCCCTGTGGGTGTCCTGGT | 1195 |
| chr3 | 58142018 | 58142038 | +/− | 21 | AGCCCTGTGGGTGTCCTGGTC | 1196 |
| chr3 | 58142019 | 58142039 | +/− | 21 | GCCCTGTGGGTGTCCTGGTCA | 1197 |
| chr3 | 58142020 | 58142040 | +/− | 21 | CCCTGTGGGTGTCCTGGTCAT | 1198 |
| chr3 | 58142021 | 58142041 | +/− | 21 | CCTGTGGGTGTCCTGGTCATT | 1199 |
| chr3 | 58142022 | 58142042 | +/− | 21 | CTGTGGGTGTCCTGGTCATTG | 1200 |
| chr3 | 58142023 | 58142043 | +/− | 21 | TGTGGGTGTCCTGGTCATTGG | 1201 |
| chr3 | 58142024 | 58142044 | +/− | 21 | GTGGGTGTCCTGGTCATTGGT | 1202 |

TABLE 8
22-mer target-specific ASOs

| | | | | | | SEQ |
|---|---|---|---|---|---|---|
| | | | | | | ID |
| CHR | START | END | STRAND | kmer | SEQUENCE | NO: |
| chr3 | 58141828 | 58141849 | +/− | 22 | CCCAACTAATCTCCATTTGCCA | 1203 |
| chr3 | 58141829 | 58141850 | +/− | 22 | CCAACTAATCTCCATTTGCCAC | 1204 |
| chr3 | 58141830 | 58141851 | +/− | 22 | CAACTAATCTCCATTTGCCACT | 1205 |
| chr3 | 58141831 | 58141852 | +/− | 22 | AACTAATCTCCATTTGCCACTG | 1206 |
| chr3 | 58141832 | 58141853 | +/− | 22 | ACTAATCTCCATTTGCCACTGA | 1207 |
| chr3 | 58141833 | 58141854 | +/− | 22 | CTAATCTCCATTTGCCACTGAC | 1208 |
| chr3 | 58141834 | 58141855 | +/− | 22 | TAATCTCCATTTGCCACTGACC | 1209 |
| chr3 | 58141835 | 58141856 | +/− | 22 | AATCTCCATTTGCCACTGACCA | 1210 |
| chr3 | 58141836 | 58141857 | +/− | 22 | ATCTCCATTTGCCACTGACCAG | 1211 |
| chr3 | 58141837 | 58141858 | +/− | 22 | TCTCCATTTGCCACTGACCAGG | 1212 |
| chr3 | 58141838 | 58141859 | +/− | 22 | CTCCATTTGCCACTGACCAGGC | 1213 |
| chr3 | 58141839 | 58141860 | +/− | 22 | TCCATTTGCCACTGACCAGGCC | 1214 |
| chr3 | 58141840 | 58141861 | +/− | 22 | CCATTTGCCACTGACCAGGCCA | 1215 |
| chr3 | 58141841 | 58141862 | +/− | 22 | CATTTGCCACTGACCAGGCCAC | 1216 |
| chr3 | 58141842 | 58141863 | +/− | 22 | ATTTGCCACTGACCAGGCCACA | 1217 |
| chr3 | 58141843 | 58141864 | +/− | 22 | TTTGCCACTGACCAGGCCACAG | 1218 |
| chr3 | 58141844 | 58141865 | +/− | 22 | TTGCCACTGACCAGGCCACAGA | 1219 |
| chr3 | 58141845 | 58141866 | +/− | 22 | TGCCACTGACCAGGCCACAGAT | 1220 |
| chr3 | 58141846 | 58141867 | +/− | 22 | GCCACTGACCAGGCCACAGATG | 1221 |
| chr3 | 58141847 | 58141868 | +/− | 22 | CCACTGACCAGGCCACAGATGG | 1222 |
| chr3 | 58141848 | 58141869 | +/− | 22 | CACTGACCAGGCCACAGATGGG | 1223 |
| chr3 | 58141849 | 58141870 | +/− | 22 | ACTGACCAGGCCACAGATGGGG | 1224 |
| chr3 | 58141850 | 58141871 | +/− | 22 | CTGACCAGGCCACAGATGGGGA | 1225 |
| chr3 | 58141851 | 58141872 | +/− | 22 | TGACCAGGCCACAGATGGGGAA | 1226 |
| chr3 | 58141852 | 58141873 | +/− | 22 | GACCAGGCCACAGATGGGGAAG | 1227 |
| chr3 | 58141853 | 58141874 | +/− | 22 | ACCAGGCCACAGATGGGGAAGT | 1228 |
| chr3 | 58141854 | 58141875 | +/− | 22 | CCAGGCCACAGATGGGGAAGTC | 1229 |
| chr3 | 58141855 | 58141876 | +/− | 22 | CAGGCCACAGATGGGGAAGTCA | 1230 |
| chr3 | 58141856 | 58141877 | +/− | 22 | AGGCCACAGATGGGGAAGTCAC | 1231 |
| chr3 | 58141857 | 58141878 | +/− | 22 | GGCCACAGATGGGGAAGTCACA | 1232 |
| chr3 | 58141858 | 58141879 | +/− | 22 | GCCACAGATGGGGAAGTCACAG | 1233 |
| chr3 | 58141859 | 58141880 | +/− | 22 | CCACAGATGGGGAAGTCACAGC | 1234 |
| chr3 | 58141860 | 58141881 | +/− | 22 | CACAGATGGGGAAGTCACAGCC | 1235 |
| chr3 | 58141861 | 58141882 | +/− | 22 | ACAGATGGGGAAGTCACAGCCG | 1236 |
| chr3 | 58141862 | 58141883 | +/− | 22 | CAGATGGGGAAGTCACAGCCGT | 1237 |
| chr3 | 58141863 | 58141884 | +/− | 22 | AGATGGGGAAGTCACAGCCGTG | 1238 |
| chr3 | 58141864 | 58141885 | +/− | 22 | GATGGGGAAGTCACAGCCGTGG | 1239 |
| chr3 | 58141865 | 58141886 | +/− | 22 | ATGGGGAAGTCACAGCCGTGGA | 1240 |
| chr3 | 58141866 | 58141887 | +/− | 22 | TGGGGAAGTCACAGCCGTGGAG | 1241 |
| chr3 | 58141867 | 58141888 | +/− | 22 | GGGGAAGTCACAGCCGTGGAGG | 1242 |
| chr3 | 58141868 | 58141889 | +/− | 22 | GGGAAGTCACAGCCGTGGAGGA | 1243 |
| chr3 | 58141869 | 58141890 | +/− | 22 | GGAAGTCACAGCCGTGGAGGAG | 1244 |
| chr3 | 58141870 | 58141891 | +/− | 22 | GAAGTCACAGCCGTGGAGGAGG | 1245 |
| chr3 | 58141871 | 58141892 | +/− | 22 | AAGTCACAGCCGTGGAGGAGGC | 1246 |
| chr3 | 58141872 | 58141893 | +/− | 22 | AGTCACAGCCGTGGAGGAGGCA | 1247 |
| chr3 | 58141873 | 58141894 | +/− | 22 | GTCACAGCCGTGGAGGAGGCAC | 1248 |
| chr3 | 58141874 | 58141895 | +/− | 22 | TCACAGCCGTGGAGGAGGCACC | 1249 |
| chr3 | 58141875 | 58141896 | +/− | 22 | CACAGCCGTGGAGGAGGCACCG | 1250 |
| chr3 | 58141876 | 58141897 | +/− | 22 | ACAGCCGTGGAGGAGGCACCGG | 1251 |
| chr3 | 58141877 | 58141898 | +/− | 22 | CAGCCGTGGAGGAGGCACCGGT | 1252 |
| chr3 | 58141878 | 58141899 | +/− | 22 | AGCCGTGGAGGAGGCACCGGTA | 1253 |
| chr3 | 58141879 | 58141900 | +/− | 22 | GCCGTGGAGGAGGCACCGGTAA | 1254 |
| chr3 | 58141880 | 58141901 | +/− | 22 | CCGTGGAGGAGGCACCGGTAAA | 1255 |
| chr3 | 58141881 | 58141902 | +/− | 22 | CGTGGAGGAGGCACCGGTAAAT | 1256 |
| chr3 | 58141882 | 58141903 | +/− | 22 | GTGGAGGAGGCACCGGTAAATG | 1257 |
| chr3 | 58141883 | 58141904 | +/− | 22 | TGGAGGAGGCACCGGTAAATGC | 1258 |
| chr3 | 58141884 | 58141905 | +/− | 22 | GGAGGAGGCACCGGTAAATGCA | 1259 |
| chr3 | 58141885 | 58141906 | +/− | 22 | GAGGAGGCACCGGTAAATGCAT | 1260 |
| chr3 | 58141886 | 58141907 | +/− | 22 | AGGAGGCACCGGTAAATGCATG | 1261 |
| chr3 | 58141887 | 58141908 | +/− | 22 | GGAGGCACCGGTAAATGCATGT | 1262 |
| chr3 | 58141888 | 58141909 | +/− | 22 | GAGGCACCGGTAAATGCATGTC | 1263 |
| chr3 | 58141889 | 58141910 | +/− | 22 | AGGCACCGGTAAATGCATGTCC | 1264 |
| chr3 | 58141890 | 58141911 | +/− | 22 | GGCACCGGTAAATGCATGTCCC | 1265 |
| chr3 | 58141891 | 58141912 | +/− | 22 | GCACCGGTAAATGCATGTCCCC | 1266 |
| chr3 | 58141892 | 58141913 | +/− | 22 | CACCGGTAAATGCATGTCCCCC | 1267 |
| chr3 | 58141893 | 58141914 | +/− | 22 | ACCGGTAAATGCATGTCCCCCT | 1268 |
| chr3 | 58141894 | 58141915 | +/− | 22 | CCGGTAAATGCATGTCCCCCTG | 1269 |
| chr3 | 58141895 | 58141916 | +/− | 22 | CGGTAAATGCATGTCCCCCTGG | 1270 |
| chr3 | 58141896 | 58141917 | +/− | 22 | GGTAAATGCATGTCCCCCTGGA | 1271 |
| chr3 | 58141897 | 58141918 | +/− | 22 | GTAAATGCATGTCCCCCTGGAT | 1272 |
| chr3 | 58141898 | 58141919 | +/− | 22 | TAAATGCATGTCCCCCTGGATT | 1273 |
| chr3 | 58141899 | 58141920 | +/− | 22 | AAATGCATGTCCCCCTGGATTC | 1274 |
| chr3 | 58141900 | 58141921 | +/− | 22 | AATGCATGTCCCCCTGGATTCA | 1275 |
| chr3 | 58141901 | 58141922 | +/− | 22 | ATGCATGTCCCCCTGGATTCAG | 1276 |
| chr3 | 58141902 | 58141923 | +/− | 22 | TGCATGTCCCCCTGGATTCAGG | 1277 |
| chr3 | 58141903 | 58141924 | +/− | 22 | GCATGTCCCCCTGGATTCAGGC | 1278 |
| chr3 | 58141904 | 58141925 | +/− | 22 | CATGTCCCCCTGGATTCAGGCC | 1279 |
| chr3 | 58141905 | 58141926 | +/− | 22 | ATGTCCCCCTGGATTCAGGCCC | 1280 |
| chr3 | 58141906 | 58141927 | +/− | 22 | TGTCCCCCTGGATTCAGGCCCT | 1281 |
| chr3 | 58141907 | 58141928 | +/− | 22 | GTCCCCCTGGATTCAGGCCCTG | 1282 |
| chr3 | 58141908 | 58141929 | +/− | 22 | TCCCCCTGGATTCAGGCCCTGG | 1283 |
| chr3 | 58141909 | 58141930 | +/− | 22 | CCCCCTGGATTCAGGCCCTGGG | 1284 |
| chr3 | 58141910 | 58141931 | +/− | 22 | CCCCTGGATTCAGGCCCTGGGT | 1285 |
| chr3 | 58141911 | 58141932 | +/− | 22 | CCCTGGATTCAGGCCCTGGGTA | 1286 |
| chr3 | 58141912 | 58141933 | +/− | 22 | CCTGGATTCAGGCCCTGGGTAC | 1287 |
| chr3 | 58141913 | 58141934 | +/− | 22 | CTGGATTCAGGCCCTGGGTACA | 1288 |
| chr3 | 58141914 | 58141935 | +/− | 22 | TGGATTCAGGCCCTGGGTACAA | 1289 |
| chr3 | 58141915 | 58141936 | +/− | 22 | GGATTCAGGCCCTGGGTACAAT | 1290 |
| chr3 | 58141916 | 58141937 | +/− | 22 | GATTCAGGCCCTGGGTACAATT | 1291 |
| chr3 | 58141917 | 58141938 | +/− | 22 | ATTCAGGCCCTGGGTACAATTT | 1292 |
| chr3 | 58141918 | 58141939 | +/− | 22 | TTCAGGCCCTGGGTACAATTTT | 1293 |
| chr3 | 58141919 | 58141940 | +/− | 22 | TCAGGCCCTGGGTACAATTTTG | 1294 |
| chr3 | 58141920 | 58141941 | +/− | 22 | CAGGCCCTGGGTACAATTTTGG | 1295 |
| chr3 | 58141921 | 58141942 | +/− | 22 | AGGCCCTGGGTACAATTTTGGT | 1296 |
| chr3 | 58141922 | 58141943 | +/− | 22 | GGCCCTGGGTACAATTTTGGTT | 1297 |
| chr3 | 58141923 | 58141944 | +/− | 22 | GCCCTGGGTACAATTTTGGTTT | 1298 |
| chr3 | 58141924 | 58141945 | +/− | 22 | CCCTGGGTACAATTTTGGTTTT | 1299 |
| chr3 | 58141925 | 58141946 | +/− | 22 | CCTGGGTACAATTTTGGTTTTT | 1300 |
| chr3 | 58141926 | 58141947 | +/− | 22 | CTGGGTACAATTTTGGTTTTTT | 1301 |
| chr3 | 58141927 | 58141948 | +/− | 22 | TGGGTACAATTTTGGTTTTTTC | 1302 |
| chr3 | 58141928 | 58141949 | +/− | 22 | GGGTACAATTTTGGTTTTTTCC | 1303 |
| chr3 | 58141929 | 58141950 | +/− | 22 | GGTACAATTTTGGTTTTTTCCT | 1304 |
| chr3 | 58141930 | 58141951 | +/− | 22 | GTACAATTTTGGTTTTTTCCTT | 1305 |
| chr3 | 58141931 | 58141952 | +/− | 22 | TACAATTTTGGTTTTTTCCTTT | 1306 |
| chr3 | 58141932 | 58141953 | +/− | 22 | ACAATTTTGGTTTTTTCCTTTT | 1307 |
| chr3 | 58141933 | 58141954 | +/− | 22 | CAATTTTGGTTTTTTCCTTTTT | 1308 |
| chr3 | 58141934 | 58141955 | +/− | 22 | AATTTTGGTTTTTTCCTTTTTG | 1309 |
| chr3 | 58141935 | 58141956 | +/− | 22 | ATTTTGGTTTTTTCCTTTTTGT | 1310 |
| chr3 | 58141936 | 58141957 | +/− | 22 | TTTTGGTTTTTTCCTTTTTGTG | 1311 |
| chr3 | 58141937 | 58141958 | +/− | 22 | TTTGGTTTTTTCCTTTTTGTGT | 1312 |
| chr3 | 58141938 | 58141959 | +/− | 22 | TTGGTTTTTTCCTTTTTGTGTT | 1313 |
| chr3 | 58141939 | 58141960 | +/− | 22 | TGGTTTTTTCCTTTTTGTGTTT | 1314 |
| chr3 | 58141940 | 58141961 | +/− | 22 | GGTTTTTTCCTTTTTGTGTTTC | 1315 |
| chr3 | 58141941 | 58141962 | +/− | 22 | GTTTTTTCCTTTTTGTGTTTCT | 1316 |
| chr3 | 58141942 | 58141963 | +/− | 22 | TTTTTTCCTTTTTGTGTTTCTG | 1317 |
| chr3 | 58141943 | 58141964 | +/− | 22 | TTTTTCCTTTTTGTGTTTCTGT | 1318 |
| chr3 | 58141944 | 58141965 | +/− | 22 | TTTTCCTTTTTGTGTTTCTGTG | 1319 |
| chr3 | 58141945 | 58141966 | +/− | 22 | TTTCCTTTTTGTGTTTCTGTGT | 1320 |
| chr3 | 58141946 | 58141967 | +/− | 22 | TTCCTTTTTGTGTTTCTGTGTT | 1321 |
| chr3 | 58141947 | 58141968 | +/− | 22 | TCCTTTTTGTGTTTCTGTGTTT | 1322 |
| chr3 | 58141948 | 58141969 | +/− | 22 | CCTTTTTGTGTTTCTGTGTTTA | 1323 |
| chr3 | 58141949 | 58141970 | +/− | 22 | CTTTTTGTGTTTCTGTGTTTAC | 1324 |
| chr3 | 58141950 | 58141971 | +/− | 22 | TTTTTGTGTTTCTGTGTTTACT | 1325 |
| chr3 | 58141951 | 58141972 | +/− | 22 | TTTTGTGTTTCTGTGTTTACTC | 1326 |
| chr3 | 58141952 | 58141973 | +/− | 22 | TTTGTGTTTCTGTGTTTACTCA | 1327 |
| chr3 | 58141953 | 58141974 | +/− | 22 | TTGTGTTTCTGTGTTTACTCAG | 1328 |
| chr3 | 58141954 | 58141975 | +/− | 22 | TGTGTTTCTGTGTTTACTCAGC | 1329 |
| chr3 | 58141955 | 58141976 | +/− | 22 | GTGTTTCTGTGTTTACTCAGCC | 1330 |
| chr3 | 58141956 | 58141977 | +/− | 22 | TGTTTCTGTGTTTACTCAGCCT | 1331 |
| chr3 | 58141957 | 58141978 | +/− | 22 | GTTTCTGTGTTTACTCAGCCTT | 1332 |
| chr3 | 58141958 | 58141979 | +/− | 22 | TTTCTGTGTTTACTCAGCCTTC | 1333 |
| chr3 | 58141959 | 58141980 | +/− | 22 | TTCTGTGTTTACTCAGCCTTCA | 1334 |
| chr3 | 58141960 | 58141981 | +/− | 22 | TCTGTGTTTACTCAGCCTTCAT | 1335 |
| chr3 | 58141961 | 58141982 | +/− | 22 | CTGTGTTTACTCAGCCTTCATT | 1336 |
| chr3 | 58141962 | 58141983 | +/− | 22 | TGTGTTTACTCAGCCTTCATTT | 1337 |
| chr3 | 58141963 | 58141984 | +/− | 22 | GTGTTTACTCAGCCTTCATTTC | 1338 |
| chr3 | 58141964 | 58141985 | +/− | 22 | TGTTTACTCAGCCTTCATTTCA | 1339 |
| chr3 | 58141965 | 58141986 | +/− | 22 | GTTTACTCAGCCTTCATTTCAG | 1340 |
| chr3 | 58141966 | 58141987 | +/− | 22 | TTTACTCAGCCTTCATTTCAGA | 1341 |
| chr3 | 58141967 | 58141988 | +/− | 22 | TTACTCAGCCTTCATTTCAGAA | 1342 |
| chr3 | 58141968 | 58141989 | +/− | 22 | TACTCAGCCTTCATTTCAGAAA | 1343 |
| chr3 | 58141969 | 58141990 | +/− | 22 | ACTCAGCCTTCATTTCAGAAAA | 1344 |
| chr3 | 58141970 | 58141991 | +/− | 22 | CTCAGCCTTCATTTCAGAAAAT | 1345 |
| chr3 | 58141971 | 58141992 | +/− | 22 | TCAGCCTTCATTTCAGAAAATC | 1346 |
| chr3 | 58141972 | 58141993 | +/− | 22 | CAGCCTTCATTTCAGAAAATCT | 1347 |
| chr3 | 58141973 | 58141994 | +/− | 22 | AGCCTTCATTTCAGAAAATCTG | 1348 |
| chr3 | 58141974 | 58141995 | +/− | 22 | GCCTTCATTTCAGAAAATCTGC | 1349 |
| chr3 | 58141975 | 58141996 | +/− | 22 | CCTTCATTTCAGAAAATCTGCC | 1350 |
| chr3 | 58141976 | 58141997 | +/− | 22 | CTTCATTTCAGAAAATCTGCCA | 1351 |
| chr3 | 58141977 | 58141998 | +/− | 22 | TTCATTTCAGAAAATCTGCCAT | 1352 |
| chr3 | 58141978 | 58141999 | +/− | 22 | TCATTTCAGAAAATCTGCCATC | 1353 |
| chr3 | 58141979 | 58142000 | +/− | 22 | CATTTCAGAAAATCTGCCATCT | 1354 |
| chr3 | 58141980 | 58142001 | +/− | 22 | ATTTCAGAAAATCTGCCATCTG | 1355 |
| chr3 | 58141981 | 58142002 | +/− | 22 | TTTCAGAAAATCTGCCATCTGC | 1356 |
| chr3 | 58141982 | 58142003 | +/− | 22 | TTCAGAAAATCTGCCATCTGCT | 1357 |
| chr3 | 58141983 | 58142004 | +/− | 22 | TCAGAAAATCTGCCATCTGCTT | 1358 |
| chr3 | 58141984 | 58142005 | +/− | 22 | CAGAAAATCTGCCATCTGCTTC | 1359 |
| chr3 | 58141985 | 58142006 | +/− | 22 | AGAAAATCTGCCATCTGCTTCT | 1360 |
| chr3 | 58141986 | 58142007 | +/− | 22 | GAAAATCTGCCATCTGCTTCTG | 1361 |
| chr3 | 58141987 | 58142008 | +/− | 22 | AAAATCTGCCATCTGCTTCTGG | 1362 |
| chr3 | 58141988 | 58142009 | +/− | 22 | AAATCTGCCATCTGCTTCTGGG | 1363 |
| chr3 | 58141989 | 58142010 | +/− | 22 | AATCTGCCATCTGCTTCTGGGA | 1364 |
| chr3 | 58141990 | 58142011 | +/− | 22 | ATCTGCCATCTGCTTCTGGGAT | 1365 |
| chr3 | 58141991 | 58142012 | +/− | 22 | TCTGCCATCTGCTTCTGGGATT | 1366 |
| chr3 | 58141992 | 58142013 | +/− | 22 | CTGCCATCTGCTTCTGGGATTG | 1367 |
| chr3 | 58141993 | 58142014 | +/− | 22 | TGCCATCTGCTTCTGGGATTGC | 1368 |
| chr3 | 58141994 | 58142015 | +/− | 22 | GCCATCTGCTTCTGGGATTGCT | 1369 |
| chr3 | 58141995 | 58142016 | +/− | 22 | CCATCTGCTTCTGGGATTGCTT | 1370 |
| chr3 | 58141996 | 58142017 | +/− | 22 | CATCTGCTTCTGGGATTGCTTA | 1371 |
| chr3 | 58141997 | 58142018 | +/− | 22 | ATCTGCTTCTGGGATTGCTTAA | 1372 |
| chr3 | 58141998 | 58142019 | +/− | 22 | TCTGCTTCTGGGATTGCTTAAG | 1373 |
| chr3 | 58141999 | 58142020 | +/− | 22 | CTGCTTCTGGGATTGCTTAAGC | 1374 |
| chr3 | 58142000 | 58142021 | +/− | 22 | TGCTTCTGGGATTGCTTAAGCC | 1375 |
| chr3 | 58142001 | 58142022 | +/− | 22 | GCTTCTGGGATTGCTTAAGCCC | 1376 |
| chr3 | 58142002 | 58142023 | +/− | 22 | CTTCTGGGATTGCTTAAGCCCT | 1377 |
| chr3 | 58142003 | 58142024 | +/− | 22 | TTCTGGGATTGCTTAAGCCCTG | 1378 |
| chr3 | 58142004 | 58142025 | +/− | 22 | TCTGGGATTGCTTAAGCCCTGT | 1379 |
| chr3 | 58142005 | 58142026 | +/− | 22 | CTGGGATTGCTTAAGCCCTGTG | 1380 |
| chr3 | 58142006 | 58142027 | +/− | 22 | TGGGATTGCTTAAGCCCTGTGG | 1381 |
| chr3 | 58142007 | 58142028 | +/− | 22 | GGGATTGCTTAAGCCCTGTGGG | 1382 |
| chr3 | 58142008 | 58142029 | +/− | 22 | GGATTGCTTAAGCCCTGTGGGT | 1383 |
| chr3 | 58142009 | 58142030 | +/− | 22 | GATTGCTTAAGCCCTGTGGGTG | 1384 |
| chr3 | 58142010 | 58142031 | +/− | 22 | ATTGCTTAAGCCCTGTGGGTGT | 1385 |
| chr3 | 58142011 | 58142032 | +/− | 22 | TTGCTTAAGCCCTGTGGGTGTC | 1386 |
| chr3 | 58142012 | 58142033 | +/− | 22 | TGCTTAAGCCCTGTGGGTGTCC | 1387 |
| chr3 | 58142013 | 58142034 | +/− | 22 | GCTTAAGCCCTGTGGGTGTCCT | 1388 |
| chr3 | 58142014 | 58142035 | +/− | 22 | CTTAAGCCCTGTGGGTGTCCTG | 1389 |
| chr3 | 58142015 | 58142036 | +/− | 22 | TTAAGCCCTGTGGGTGTCCTGG | 1390 |
| chr3 | 58142016 | 58142037 | +/− | 22 | TAAGCCCTGTGGGTGTCCTGGT | 1391 |
| chr3 | 58142017 | 58142038 | +/− | 22 | AAGCCCTGTGGGTGTCCTGGTC | 1392 |
| chr3 | 58142018 | 58142039 | +/− | 22 | AGCCCTGTGGGTGTCCTGGTCA | 1393 |
| chr3 | 58142019 | 58142040 | +/− | 22 | GCCCTGTGGGTGTCCTGGTCAT | 1394 |
| chr3 | 58142020 | 58142041 | +/− | 22 | CCCTGTGGGTGTCCTGGTCATT | 1395 |
| chr3 | 58142021 | 58142042 | +/− | 22 | CCTGTGGGTGTCCTGGTCATTG | 1396 |
| chr3 | 58142022 | 58142043 | +/− | 22 | CTGTGGGTGTCCTGGTCATTGG | 1397 |
| chr3 | 58142023 | 58142044 | +/− | 22 | TGTGGGTGTCCTGGTCATTGGT | 1398 |

Tables 9-15 comprise some additional example oligonucleotide sequences of variable sequence lengths that may be used to induce an isoform switch or modulate (e.g., inhibit or enhance) the biological activity of a specific isoform of one or several of the genes described above or elsewhere herein (e.g., genes comprising one or more of NEDD4L (ENV2), MAP3K7 (ENV3), NFYA (ENV11), ESYT2 (ENV21), MARK2 (ENV18), ST7 (ENV19), ARVCF (ENV22), SYTL2 (ENV17), R3HDM1 (ENV23), COL4A3BP (ENV9), TANGO2 (ENV6), SEPT9 (ENV15), ROBO1 (ENV4), FAM122B (ENV5), CD47 (ENV13), LSR (ENV20), PBX1 (ENV16), EPB41 (ENV14), ADAM15 (ENV7), EPB41L1 (ENV8), ABI1 (ENV10), FLNB (ENV1), CTNND1 (ENV12), GPR160 (ENV24), ITGB3BP (ENV25), INCENP (ENV26), DENND1B (ENV27), CA12 (ENV28).

As discussed above, oligonucleotide sequences comprised in a table may be specific for a single target, or for more than one target. In some cases, oligonucleotide sequences comprised in more than one tables are specific for a single target. For example, oligonucleotide sequences comprised in Tables 9-15 may be specific for a single target. The target may be the same as or differ from the target which the oligonucleotide sequences comprised in Tables 2-8 are specific for. The target may be a gene selected from genes described above or elsewhere herein.

TABLE 9
16-mer target-specific

| | | | | | | SEQ |
|---|---|---|---|---|---|---|
| | | | | | | ID |
| CHR | START | END | STRAND | Kmer | SEQUENCE | NO: |
| chr18 | 58335318 | 58335333 | +/− | 16 | GGTCATCAGCGACTGC | 1399 |
| chr18 | 58335319 | 58335334 | +/− | 16 | GTCATCAGCGACTGCT | 1400 |
| chr18 | 58335320 | 58335335 | +/− | 16 | TCATCAGCGACTGCTG | 1401 |
| chr18 | 58335321 | 58335336 | +/− | 16 | CATCAGCGACTGCTGG | 1402 |
| chr18 | 58335322 | 58335337 | +/− | 16 | ATCAGCGACTGCTGGC | 1403 |
| chr18 | 58335323 | 58335338 | +/− | 16 | TCAGCGACTGCTGGCT | 1404 |
| chr18 | 58335324 | 58335339 | +/− | 16 | CAGCGACTGCTGGCTT | 1405 |
| chr18 | 58335325 | 58335340 | +/− | 16 | AGCGACTGCTGGCTTT | 1406 |
| chr18 | 58335326 | 58335341 | +/− | 16 | GCGACTGCTGGCTTTG | 1407 |
| chr18 | 58335327 | 58335342 | +/− | 16 | CGACTGCTGGCTTTGT | 1408 |
| chr18 | 58335328 | 58335343 | +/− | 16 | GACTGCTGGCTTTGTC | 1409 |
| chr18 | 58335329 | 58335344 | +/− | 16 | ACTGCTGGCTTTGTCT | 1410 |
| chr18 | 58335330 | 58335345 | +/− | 16 | CTGCTGGCTTTGTCTG | 1411 |
| chr18 | 58335331 | 58335346 | +/− | 16 | TGCTGGCTTTGTCTGG | 1412 |
| chr18 | 58335332 | 58335347 | +/− | 16 | GCTGGCTTTGTCTGGA | 1413 |
| chr18 | 58335333 | 58335348 | +/− | 16 | CTGGCTTTGTCTGGAT | 1414 |
| chr18 | 58335334 | 58335349 | +/− | 16 | TGGCTTTGTCTGGATA | 1415 |
| chr18 | 58335335 | 58335350 | +/− | 16 | GGCTTTGTCTGGATAG | 1416 |
| chr18 | 58335336 | 58335351 | +/− | 16 | GCTTTGTCTGGATAGG | 1417 |
| chr18 | 58335337 | 58335352 | +/− | 16 | CTTTGTCTGGATAGGG | 1418 |
| chr18 | 58335338 | 58335353 | +/− | 16 | TTTGTCTGGATAGGGT | 1419 |
| chr18 | 58335339 | 58335354 | +/− | 16 | TTGTCTGGATAGGGTG | 1420 |
| chr18 | 58335340 | 58335355 | +/− | 16 | TGTCTGGATAGGGTGG | 1421 |
| chr18 | 58335341 | 58335356 | +/− | 16 | GTCTGGATAGGGTGGG | 1422 |
| chr18 | 58335342 | 58335357 | +/− | 16 | TCTGGATAGGGTGGGT | 1423 |
| chr18 | 58335343 | 58335358 | +/− | 16 | CTGGATAGGGTGGGTT | 1424 |
| chr18 | 58335344 | 58335359 | +/− | 16 | TGGATAGGGTGGGTTT | 1425 |
| chr18 | 58335345 | 58335360 | +/− | 16 | GGATAGGGTGGGTTTC | 1426 |
| chr18 | 58335346 | 58335361 | +/− | 16 | GATAGGGTGGGTTTCA | 1427 |
| chr18 | 58335347 | 58335362 | +/− | 16 | ATAGGGTGGGTTTCAG | 1428 |
| chr18 | 58335348 | 58335363 | +/− | 16 | TAGGGTGGGTTTCAGG | 1429 |
| chr18 | 58335349 | 58335364 | +/− | 16 | AGGGTGGGTTTCAGGG | 1430 |
| chr18 | 58335350 | 58335365 | +/− | 16 | GGGTGGGTTTCAGGGA | 1431 |
| chr18 | 58335351 | 58335366 | +/− | 16 | GGTGGGTTTCAGGGAT | 1432 |
| chr18 | 58335352 | 58335367 | +/− | 16 | GTGGGTTTCAGGGATT | 1433 |
| chr18 | 58335353 | 58335368 | +/− | 16 | TGGGTTTCAGGGATTC | 1434 |
| chr18 | 58335354 | 58335369 | +/− | 16 | GGGTTTCAGGGATTCT | 1435 |
| chr18 | 58335355 | 58335370 | +/− | 16 | GGTTTCAGGGATTCTG | 1436 |
| chr18 | 58335356 | 58335371 | +/− | 16 | GTTTCAGGGATTCTGA | 1437 |
| chr18 | 58335357 | 58335372 | +/− | 16 | TTTCAGGGATTCTGAT | 1438 |
| chr18 | 58335358 | 58335373 | +/− | 16 | TTCAGGGATTCTGATC | 1439 |
| chr18 | 58335359 | 58335374 | +/− | 16 | TCAGGGATTCTGATCT | 1440 |
| chr18 | 58335360 | 58335375 | +/− | 16 | CAGGGATTCTGATCTC | 1441 |
| chr18 | 58335361 | 58335376 | +/− | 16 | AGGGATTCTGATCTCA | 1442 |
| chr18 | 58335362 | 58335377 | +/− | 16 | GGGATTCTGATCTCAC | 1443 |
| chr18 | 58335363 | 58335378 | +/− | 16 | GGATTCTGATCTCACG | 1444 |
| chr18 | 58335364 | 58335379 | +/− | 16 | GATTCTGATCTCACGT | 1445 |
| chr18 | 58335365 | 58335380 | +/− | 16 | ATTCTGATCTCACGTC | 1446 |
| chr18 | 58335366 | 58335381 | +/− | 16 | TTCTGATCTCACGTCA | 1447 |
| chr18 | 58335367 | 58335382 | +/− | 16 | TCTGATCTCACGTCAC | 1448 |
| chr18 | 58335368 | 58335383 | +/− | 16 | CTGATCTCACGTCACC | 1449 |
| chr18 | 58335369 | 58335384 | +/− | 16 | TGATCTCACGTCACCT | 1450 |
| chr18 | 58335370 | 58335385 | +/− | 16 | GATCTCACGTCACCTG | 1451 |
| chr18 | 58335371 | 58335386 | +/− | 16 | ATCTCACGTCACCTGC | 1452 |
| chr18 | 58335372 | 58335387 | +/− | 16 | TCTCACGTCACCTGCC | 1453 |
| chr18 | 58335373 | 58335388 | +/− | 16 | CTCACGTCACCTGCCT | 1454 |
| chr18 | 58335374 | 58335389 | +/− | 16 | TCACGTCACCTGCCTT | 1455 |
| chr18 | 58335375 | 58335390 | +/− | 16 | CACGTCACCTGCCTTA | 1456 |
| chr18 | 58335376 | 58335391 | +/− | 16 | ACGTCACCTGCCTTAC | 1457 |
| chr18 | 58335377 | 58335392 | +/− | 16 | CGTCACCTGCCTTACA | 1458 |
| chr18 | 58335378 | 58335393 | +/− | 16 | GTCACCTGCCTTACAG | 1459 |
| chr18 | 58335379 | 58335394 | +/− | 16 | TCACCTGCCTTACAGC | 1460 |
| chr18 | 58335380 | 58335395 | +/− | 16 | CACCTGCCTTACAGCG | 1461 |
| chr18 | 58335381 | 58335396 | +/− | 16 | ACCTGCCTTACAGCGC | 1462 |
| chr18 | 58335382 | 58335397 | +/− | 16 | CCTGCCTTACAGCGCT | 1463 |
| chr18 | 58335383 | 58335398 | +/− | 16 | CTGCCTTACAGCGCTG | 1464 |
| chr18 | 58335384 | 58335399 | +/− | 16 | TGCCTTACAGCGCTGC | 1465 |
| chr18 | 58335385 | 58335400 | +/− | 16 | GCCTTACAGCGCTGCC | 1466 |
| chr18 | 58335386 | 58335401 | +/− | 16 | CCTTACAGCGCTGCCA | 1467 |
| chr18 | 58335387 | 58335402 | +/− | 16 | CTTACAGCGCTGCCAC | 1468 |
| chr18 | 58335388 | 58335403 | +/− | 16 | TTACAGCGCTGCCACA | 1469 |
| chr18 | 58335389 | 58335404 | +/− | 16 | TACAGCGCTGCCACAG | 1470 |
| chr18 | 58335390 | 58335405 | +/− | 16 | ACAGCGCTGCCACAGC | 1471 |
| chr18 | 58335391 | 58335406 | +/− | 16 | CAGCGCTGCCACAGCA | 1472 |
| chr18 | 58335392 | 58335407 | +/− | 16 | AGCGCTGCCACAGCAG | 1473 |
| chr18 | 58335393 | 58335408 | +/− | 16 | GCGCTGCCACAGCAGT | 1474 |
| chr18 | 58335394 | 58335409 | +/− | 16 | CGCTGCCACAGCAGTG | 1475 |
| chr18 | 58335395 | 58335410 | +/− | 16 | GCTGCCACAGCAGTGG | 1476 |
| chr18 | 58335396 | 58335411 | +/− | 16 | CTGCCACAGCAGTGGG | 1477 |
| chr18 | 58335397 | 58335412 | +/− | 16 | TGCCACAGCAGTGGGC | 1478 |
| chr18 | 58335398 | 58335413 | +/− | 16 | GCCACAGCAGTGGGCC | 1479 |
| chr18 | 58335399 | 58335414 | +/− | 16 | CCACAGCAGTGGGCCC | 1480 |
| chr18 | 58335400 | 58335415 | +/− | 16 | CACAGCAGTGGGCCCT | 1481 |
| chr18 | 58335401 | 58335416 | +/− | 16 | ACAGCAGTGGGCCCTG | 1482 |
| chr18 | 58335402 | 58335417 | +/− | 16 | CAGCAGTGGGCCCTGA | 1483 |
| chr18 | 58335403 | 58335418 | +/− | 16 | AGCAGTGGGCCCTGAT | 1484 |
| chr18 | 58335404 | 58335419 | +/− | 16 | GCAGTGGGCCCTGATT | 1485 |
| chr18 | 58335405 | 58335420 | +/− | 16 | CAGTGGGCCCTGATTC | 1486 |
| chr18 | 58335406 | 58335421 | +/− | 16 | AGTGGGCCCTGATTCA | 1487 |
| chr18 | 58335407 | 58335422 | +/− | 16 | GTGGGCCCTGATTCAG | 1488 |
| chr18 | 58335408 | 58335423 | +/− | 16 | TGGGCCCTGATTCAGA | 1489 |
| chr18 | 58335409 | 58335424 | +/− | 16 | GGGCCCTGATTCAGAC | 1490 |
| chr18 | 58335410 | 58335425 | +/− | 16 | GGCCCTGATTCAGACA | 1491 |
| chr18 | 58335411 | 58335426 | +/− | 16 | GCCCTGATTCAGACAG | 1492 |
| chr18 | 58335412 | 58335427 | +/− | 16 | CCCTGATTCAGACAGC | 1493 |
| chr18 | 58335413 | 58335428 | +/− | 16 | CCTGATTCAGACAGCA | 1494 |
| chr18 | 58335414 | 58335429 | +/− | 16 | CTGATTCAGACAGCAG | 1495 |
| chr18 | 58335415 | 58335430 | +/− | 16 | TGATTCAGACAGCAGG | 1496 |
| chr18 | 58335317 | 58335332 | +/− | 16 | TGGTCATCAGCGACTG | 1497 |
| chr18 | 58335416 | 58335431 | +/− | 16 | GATTCAGACAGCAGGG | 1498 |
| chr18 | 58335417 | 58335432 | +/− | 16 | ATTCAGACAGCAGGGG | 1499 |
| chr18 | 58335418 | 58335433 | +/− | 16 | TTCAGACAGCAGGGGG | 1500 |
| chr18 | 58335419 | 58335434 | +/− | 16 | TCAGACAGCAGGGGGT | 1501 |
| chr18 | 58335420 | 58335435 | +/− | 16 | CAGACAGCAGGGGGTC | 1502 |
| chr18 | 58335421 | 58335436 | +/− | 16 | AGACAGCAGGGGGTCA | 1503 |
| chr18 | 58335422 | 58335437 | +/− | 16 | GACAGCAGGGGGTCAT | 1504 |
| chr18 | 58335423 | 58335438 | +/− | 16 | ACAGCAGGGGGTCATC | 1505 |
| chr18 | 58335424 | 58335439 | +/− | 16 | CAGCAGGGGGTCATCC | 1506 |
| chr18 | 58335425 | 58335440 | +/− | 16 | AGCAGGGGGTCATCCC | 1507 |
| chr18 | 58335426 | 58335441 | +/− | 16 | GCAGGGGGTCATCCCC | 1508 |
| chr18 | 58335427 | 58335442 | +/− | 16 | CAGGGGGTCATCCCCT | 1509 |
| chr18 | 58335428 | 58335443 | +/− | 16 | AGGGGGTCATCCCCTA | 1510 |
| chr18 | 58335429 | 58335444 | +/− | 16 | GGGGGTCATCCCCTAA | 1511 |
| chr18 | 58335430 | 58335445 | +/− | 16 | GGGGTCATCCCCTAAG | 1512 |
| chr18 | 58335431 | 58335446 | +/− | 16 | GGGTCATCCCCTAAGT | 1513 |
| chr18 | 58335432 | 58335447 | +/− | 16 | GGTCATCCCCTAAGTG | 1514 |

TABLE 10
17-mer target-specific ASOs

| | | | | | | SEQ |
|---|---|---|---|---|---|---|
| | | | | | | ID |
| CHR | START | END | STRAND | Kmer | SEQUENCE | NO: |
| chr18 | 58335318 | 58335334 | +/− | 17 | GGTCATCAGCGACTGCT | 1515 |
| chr18 | 58335319 | 58335335 | +/− | 17 | GTCATCAGCGACTGCTG | 1516 |
| chr18 | 58335320 | 58335336 | +/− | 17 | TCATCAGCGACTGCTGG | 1517 |
| chr18 | 58335321 | 58335337 | +/− | 17 | CATCAGCGACTGCTGGC | 1518 |
| chr18 | 58335322 | 58335338 | +/− | 17 | ATCAGCGACTGCTGGCT | 1519 |
| chr18 | 58335323 | 58335339 | +/− | 17 | TCAGCGACTGCTGGCTT | 1520 |
| chr18 | 58335324 | 58335340 | +/− | 17 | CAGCGACTGCTGGCTTT | 1521 |
| chr18 | 58335325 | 58335341 | +/− | 17 | AGCGACTGCTGGCTTTG | 1522 |
| chr18 | 58335326 | 58335342 | +/− | 17 | GCGACTGCTGGCTTTGT | 1523 |
| chr18 | 58335327 | 58335343 | +/− | 17 | CGACTGCTGGCTTTGTC | 1524 |
| chr18 | 58335328 | 58335344 | +/− | 17 | GACTGCTGGCTTTGTCT | 1525 |
| chr18 | 58335329 | 58335345 | +/− | 17 | ACTGCTGGCTTTGTCTG | 1526 |
| chr18 | 58335330 | 58335346 | +/− | 17 | CTGCTGGCTTTGTCTGG | 1527 |
| chr18 | 58335331 | 58335347 | +/− | 17 | TGCTGGCTTTGTCTGGA | 1528 |
| chr18 | 58335332 | 58335348 | +/− | 17 | GCTGGCTTTGTCTGGAT | 1529 |
| chr18 | 58335333 | 58335349 | +/− | 17 | CTGGCTTTGTCTGGATA | 1530 |
| chr18 | 58335334 | 58335350 | +/− | 17 | TGGCTTTGTCTGGATAG | 1531 |
| chr18 | 58335335 | 58335351 | +/− | 17 | GGCTTTGTCTGGATAGG | 1532 |
| chr18 | 58335336 | 58335352 | +/− | 17 | GCTTTGTCTGGATAGGG | 1533 |
| chr18 | 58335337 | 58335353 | +/− | 17 | CTTTGTCTGGATAGGGT | 1534 |
| chr18 | 58335338 | 58335354 | +/− | 17 | TTTGTCTGGATAGGGTG | 1535 |
| chr18 | 58335339 | 58335355 | +/− | 17 | TTGTCTGGATAGGGTGG | 1536 |
| chr18 | 58335340 | 58335356 | +/− | 17 | TGTCTGGATAGGGTGGG | 1537 |
| chr18 | 58335341 | 58335357 | +/− | 17 | GTCTGGATAGGGTGGGT | 1538 |
| chr18 | 58335342 | 58335358 | +/− | 17 | TCTGGATAGGGTGGGTT | 1539 |
| chr18 | 58335343 | 58335359 | +/− | 17 | CTGGATAGGGTGGGTTT | 1540 |
| chr18 | 58335344 | 58335360 | +/− | 17 | TGGATAGGGTGGGTTTC | 1541 |
| chr18 | 58335345 | 58335361 | +/− | 17 | GGATAGGGTGGGTTTCA | 1542 |
| chr18 | 58335346 | 58335362 | +/− | 17 | GATAGGGTGGGTTTCAG | 1543 |
| chr18 | 58335347 | 58335363 | +/− | 17 | ATAGGGTGGGTTTCAGG | 1544 |
| chr18 | 58335348 | 58335364 | +/− | 17 | TAGGGTGGGTTTCAGGG | 1545 |
| chr18 | 58335349 | 58335365 | +/− | 17 | AGGGTGGGTTTCAGGGA | 1546 |
| chr18 | 58335350 | 58335366 | +/− | 17 | GGGTGGGTTTCAGGGAT | 1547 |
| chr18 | 58335351 | 58335367 | +/− | 17 | GGTGGGTTTCAGGGATT | 1548 |
| chr18 | 58335352 | 58335368 | +/− | 17 | GTGGGTTTCAGGGATTC | 1549 |
| chr18 | 58335353 | 58335369 | +/− | 17 | TGGGTTTCAGGGATTCT | 1550 |
| chr18 | 58335354 | 58335370 | +/− | 17 | GGGTTTCAGGGATTCTG | 1551 |
| chr18 | 58335355 | 58335371 | +/− | 17 | GGTTTCAGGGATTCTGA | 1552 |
| chr18 | 58335356 | 58335372 | +/− | 17 | GTTTCAGGGATTCTGAT | 1553 |
| chr18 | 58335357 | 58335373 | +/− | 17 | TTTCAGGGATTCTGATC | 1554 |
| chr18 | 58335358 | 58335374 | +/− | 17 | TTCAGGGATTCTGATCT | 1555 |
| chr18 | 58335359 | 58335375 | +/− | 17 | TCAGGGATTCTGATCTC | 1556 |
| chr18 | 58335360 | 58335376 | +/− | 17 | CAGGGATTCTGATCTCA | 1557 |
| chr18 | 58335361 | 58335377 | +/− | 17 | AGGGATTCTGATCTCAC | 1558 |
| chr18 | 58335362 | 58335378 | +/− | 17 | GGGATTCTGATCTCACG | 1559 |
| chr18 | 58335363 | 58335379 | +/− | 17 | GGATTCTGATCTCACGT | 1560 |
| chr18 | 58335364 | 58335380 | +/− | 17 | GATTCTGATCTCACGTC | 1561 |
| chr18 | 58335365 | 58335381 | +/− | 17 | ATTCTGATCTCACGTCA | 1562 |
| chr18 | 58335366 | 58335382 | +/− | 17 | TTCTGATCTCACGTCAC | 1563 |
| chr18 | 58335367 | 58335383 | +/− | 17 | TCTGATCTCACGTCACC | 1564 |
| chr18 | 58335368 | 58335384 | +/− | 17 | CTGATCTCACGTCACCT | 1565 |
| chr18 | 58335369 | 58335385 | +/− | 17 | TGATCTCACGTCACCTG | 1566 |
| chr18 | 58335370 | 58335386 | +/− | 17 | GATCTCACGTCACCTGC | 1567 |
| chr18 | 58335371 | 58335387 | +/− | 17 | ATCTCACGTCACCTGCC | 1568 |
| chr18 | 58335372 | 58335388 | +/− | 17 | TCTCACGTCACCTGCCT | 1569 |
| chr18 | 58335373 | 58335389 | +/− | 17 | CTCACGTCACCTGCCTT | 1570 |
| chr18 | 58335374 | 58335390 | +/− | 17 | TCACGTCACCTGCCTTA | 1571 |
| chr18 | 58335375 | 58335391 | +/− | 17 | CACGTCACCTGCCTTAC | 1572 |
| chr18 | 58335376 | 58335392 | +/− | 17 | ACGTCACCTGCCTTACA | 1573 |
| chr18 | 58335377 | 58335393 | +/− | 17 | CGTCACCTGCCTTACAG | 1574 |
| chr18 | 58335378 | 58335394 | +/− | 17 | GTCACCTGCCTTACAGC | 1575 |
| chr18 | 58335379 | 58335395 | +/− | 17 | TCACCTGCCTTACAGCG | 1576 |
| chr18 | 58335380 | 58335396 | +/− | 17 | CACCTGCCTTACAGCGC | 1577 |
| chr18 | 58335381 | 58335397 | +/− | 17 | ACCTGCCTTACAGCGCT | 1578 |
| chr18 | 58335382 | 58335398 | +/− | 17 | CCTGCCTTACAGCGCTG | 1579 |
| chr18 | 58335383 | 58335399 | +/− | 17 | CTGCCTTACAGCGCTGC | 1580 |
| chr18 | 58335384 | 58335400 | +/− | 17 | TGCCTTACAGCGCTGCC | 1581 |
| chr18 | 58335385 | 58335401 | +/− | 17 | GCCTTACAGCGCTGCCA | 1582 |
| chr18 | 58335386 | 58335402 | +/− | 17 | CCTTACAGCGCTGCCAC | 1583 |
| chr18 | 58335387 | 58335403 | +/− | 17 | CTTACAGCGCTGCCACA | 1584 |
| chr18 | 58335388 | 58335404 | +/− | 17 | TTACAGCGCTGCCACAG | 1585 |
| chr18 | 58335389 | 58335405 | +/− | 17 | TACAGCGCTGCCACAGC | 1586 |
| chr18 | 58335390 | 58335406 | +/− | 17 | ACAGCGCTGCCACAGCA | 1587 |
| chr18 | 58335391 | 58335407 | +/− | 17 | CAGCGCTGCCACAGCAG | 1588 |
| chr18 | 58335392 | 58335408 | +/− | 17 | AGCGCTGCCACAGCAGT | 1589 |
| chr18 | 58335393 | 58335409 | +/− | 17 | GCGCTGCCACAGCAGTG | 1590 |
| chr18 | 58335394 | 58335410 | +/− | 17 | CGCTGCCACAGCAGTGG | 1591 |
| chr18 | 58335395 | 58335411 | +/− | 17 | GCTGCCACAGCAGTGGG | 1592 |
| chr18 | 58335396 | 58335412 | +/− | 17 | CTGCCACAGCAGTGGGC | 1593 |
| chr18 | 58335397 | 58335413 | +/− | 17 | TGCCACAGCAGTGGGCC | 1594 |
| chr18 | 58335398 | 58335414 | +/− | 17 | GCCACAGCAGTGGGCCC | 1595 |
| chr18 | 58335399 | 58335415 | +/− | 17 | CCACAGCAGTGGGCCCT | 1596 |
| chr18 | 58335400 | 58335416 | +/− | 17 | CACAGCAGTGGGCCCTG | 1597 |
| chr18 | 58335401 | 58335417 | +/− | 17 | ACAGCAGTGGGCCCTGA | 1598 |
| chr18 | 58335402 | 58335418 | +/− | 17 | CAGCAGTGGGCCCTGAT | 1599 |
| chr18 | 58335403 | 58335419 | +/− | 17 | AGCAGTGGGCCCTGATT | 1600 |
| chr18 | 58335404 | 58335420 | +/− | 17 | GCAGTGGGCCCTGATTC | 1601 |
| chr18 | 58335405 | 58335421 | +/− | 17 | CAGTGGGCCCTGATTCA | 1602 |
| chr18 | 58335406 | 58335422 | +/− | 17 | AGTGGGCCCTGATTCAG | 1603 |
| chr18 | 58335407 | 58335423 | +/− | 17 | GTGGGCCCTGATTCAGA | 1604 |
| chr18 | 58335408 | 58335424 | +/− | 17 | TGGGCCCTGATTCAGAC | 1605 |
| chr18 | 58335409 | 58335425 | +/− | 17 | GGGCCCTGATTCAGACA | 1606 |
| chr18 | 58335410 | 58335426 | +/− | 17 | GGCCCTGATTCAGACAG | 1607 |
| chr18 | 58335411 | 58335427 | +/− | 17 | GCCCTGATTCAGACAGC | 1608 |
| chr18 | 58335412 | 58335428 | +/− | 17 | CCCTGATTCAGACAGCA | 1609 |
| chr18 | 58335413 | 58335429 | +/− | 17 | CCTGATTCAGACAGCAG | 1610 |
| chr18 | 58335414 | 58335430 | +/− | 17 | CTGATTCAGACAGCAGG | 1611 |
| chr18 | 58335317 | 58335333 | +/− | 17 | TGGTCATCAGCGACTGC | 1612 |
| chr18 | 58335415 | 58335431 | +/− | 17 | TGATTCAGACAGCAGGG | 1613 |
| chr18 | 58335416 | 58335432 | +/− | 17 | GATTCAGACAGCAGGGG | 1614 |
| chr18 | 58335417 | 58335433 | +/− | 17 | ATTCAGACAGCAGGGGG | 1615 |
| chr18 | 58335418 | 58335434 | +/− | 17 | TTCAGACAGCAGGGGGT | 1616 |
| chr18 | 58335419 | 58335435 | +/− | 17 | TCAGACAGCAGGGGGTC | 1617 |
| chr18 | 58335420 | 58335436 | +/− | 17 | CAGACAGCAGGGGGTCA | 1618 |
| chr18 | 58335421 | 58335437 | +/− | 17 | AGACAGCAGGGGGTCAT | 1619 |
| chr18 | 58335422 | 58335438 | +/− | 17 | GACAGCAGGGGGTCATC | 1620 |
| chr18 | 58335423 | 58335439 | +/− | 17 | ACAGCAGGGGGTCATCC | 1621 |
| chr18 | 58335424 | 58335440 | +/− | 17 | CAGCAGGGGGTCATCCC | 1622 |
| chr18 | 58335425 | 58335441 | +/− | 17 | AGCAGGGGGTCATCCCC | 1623 |
| chr18 | 58335426 | 58335442 | +/− | 17 | GCAGGGGGTCATCCCCT | 1624 |
| chr18 | 58335427 | 58335443 | +/− | 17 | CAGGGGGTCATCCCCTA | 1625 |
| chr18 | 58335428 | 58335444 | +/− | 17 | AGGGGGTCATCCCCTAA | 1626 |
| chr18 | 58335429 | 58335445 | +/− | 17 | GGGGGTCATCCCCTAAG | 1627 |
| chr18 | 58335430 | 58335446 | +/− | 17 | GGGGTCATCCCCTAAGT | 1628 |
| chr18 | 58335431 | 58335447 | +/− | 17 | GGGTCATCCCCTAAGTG | 1629 |

TABLE 11
18-mer target-specific ASOs

| | | | | | | SEQ |
|---|---|---|---|---|---|---|
| | | | | | | ID |
| CHR | START | END | STRAND | Kmer | SEQUENCE | NO: |
| chr18 | 58335318 | 58335335 | +/− | 18 | GGTCATCAGCGACTGCTG | 1630 |
| chr18 | 58335319 | 58335336 | +/− | 18 | GTCATCAGCGACTGCTGG | 1631 |
| chr18 | 58335320 | 58335337 | +/− | 18 | TCATCAGCGACTGCTGGC | 1632 |
| chr18 | 58335321 | 58335338 | +/− | 18 | CATCAGCGACTGCTGGCT | 1633 |
| chr18 | 58335322 | 58335339 | +/− | 18 | ATCAGCGACTGCTGGCTT | 1634 |
| chr18 | 58335323 | 58335340 | +/− | 18 | TCAGCGACTGCTGGCTTT | 1635 |
| chr18 | 58335324 | 58335341 | +/− | 18 | CAGCGACTGCTGGCTTTG | 1636 |
| chr18 | 58335325 | 58335342 | +/− | 18 | AGCGACTGCTGGCTTTGT | 1637 |
| chr18 | 58335326 | 58335343 | +/− | 18 | GCGACTGCTGGCTTTGTC | 1638 |
| chr18 | 58335327 | 58335344 | +/− | 18 | CGACTGCTGGCTTTGTCT | 1639 |
| chr18 | 58335328 | 58335345 | +/− | 18 | GACTGCTGGCTTTGTCTG | 1640 |
| chr18 | 58335329 | 58335346 | +/− | 18 | ACTGCTGGCTTTGTCTGG | 1641 |
| chr18 | 58335330 | 58335347 | +/− | 18 | CTGCTGGCTTTGTCTGGA | 1642 |
| chr18 | 58335331 | 58335348 | +/− | 18 | TGCTGGCTTTGTCTGGAT | 1643 |
| chr18 | 58335332 | 58335349 | +/− | 18 | GCTGGCTTTGTCTGGATA | 1644 |
| chr18 | 58335333 | 58335350 | +/− | 18 | CTGGCTTTGTCTGGATAG | 1645 |
| chr18 | 58335334 | 58335351 | +/− | 18 | TGGCTTTGTCTGGATAGG | 1646 |
| chr18 | 58335335 | 58335352 | +/− | 18 | GGCTTTGTCTGGATAGGG | 1647 |
| chr18 | 58335336 | 58335353 | +/− | 18 | GCTTTGTCTGGATAGGGT | 1648 |
| chr18 | 58335337 | 58335354 | +/− | 18 | CTTTGTCTGGATAGGGTG | 1649 |
| chr18 | 58335338 | 58335355 | +/− | 18 | TTTGTCTGGATAGGGTGG | 1650 |
| chr18 | 58335339 | 58335356 | +/− | 18 | TTGTCTGGATAGGGTGGG | 1651 |
| chr18 | 58335340 | 58335357 | +/− | 18 | TGTCTGGATAGGGTGGGT | 1652 |
| chr18 | 58335341 | 58335358 | +/− | 18 | GTCTGGATAGGGTGGGTT | 1653 |
| chr18 | 58335342 | 58335359 | +/− | 18 | TCTGGATAGGGTGGGTTT | 1654 |
| chr18 | 58335343 | 58335360 | +/− | 18 | CTGGATAGGGTGGGTTTC | 1655 |
| chr18 | 58335344 | 58335361 | +/− | 18 | TGGATAGGGTGGGTTTCA | 1656 |
| chr18 | 58335345 | 58335362 | +/− | 18 | GGATAGGGTGGGTTTCAG | 1657 |
| chr18 | 58335346 | 58335363 | +/− | 18 | GATAGGGTGGGTTTCAGG | 1658 |
| chr18 | 58335347 | 58335364 | +/− | 18 | ATAGGGTGGGTTTCAGGG | 1659 |
| chr18 | 58335348 | 58335365 | +/− | 18 | TAGGGTGGGTTTCAGGGA | 1660 |
| chr18 | 58335349 | 58335366 | +/− | 18 | AGGGTGGGTTTCAGGGAT | 1661 |
| chr18 | 58335350 | 58335367 | +/− | 18 | GGGTGGGTTTCAGGGATT | 1662 |
| chr18 | 58335351 | 58335368 | +/− | 18 | GGTGGGTTTCAGGGATTC | 1663 |
| chr18 | 58335352 | 58335369 | +/− | 18 | GTGGGTTTCAGGGATTCT | 1664 |
| chr18 | 58335353 | 58335370 | +/− | 18 | TGGGTTTCAGGGATTCTG | 1665 |
| chr18 | 58335354 | 58335371 | +/− | 18 | GGGTTTCAGGGATTCTGA | 1666 |
| chr18 | 58335355 | 58335372 | +/− | 18 | GGTTTCAGGGATTCTGAT | 1667 |
| chr18 | 58335356 | 58335373 | +/− | 18 | GTTTCAGGGATTCTGATC | 1668 |
| chr18 | 58335357 | 58335374 | +/− | 18 | TTTCAGGGATTCTGATCT | 1669 |
| chr18 | 58335358 | 58335375 | +/− | 18 | TTCAGGGATTCTGATCTC | 1670 |
| chr18 | 58335359 | 58335376 | +/− | 18 | TCAGGGATTCTGATCTCA | 1671 |
| chr18 | 58335360 | 58335377 | +/− | 18 | CAGGGATTCTGATCTCAC | 1672 |
| chr18 | 58335361 | 58335378 | +/− | 18 | AGGGATTCTGATCTCACG | 1673 |
| chr18 | 58335362 | 58335379 | +/− | 18 | GGGATTCTGATCTCACGT | 1674 |
| chr18 | 58335363 | 58335380 | +/− | 18 | GGATTCTGATCTCACGTC | 1675 |
| chr18 | 58335364 | 58335381 | +/− | 18 | GATTCTGATCTCACGTCA | 1676 |
| chr18 | 58335365 | 58335382 | +/− | 18 | ATTCTGATCTCACGTCAC | 1677 |
| chr18 | 58335366 | 58335383 | +/− | 18 | TTCTGATCTCACGTCACC | 1678 |
| chr18 | 58335367 | 58335384 | +/− | 18 | TCTGATCTCACGTCACCT | 1679 |
| chr18 | 58335368 | 58335385 | +/− | 18 | CTGATCTCACGTCACCTG | 1680 |
| chr18 | 58335369 | 58335386 | +/− | 18 | TGATCTCACGTCACCTGC | 1681 |
| chr18 | 58335370 | 58335387 | +/− | 18 | GATCTCACGTCACCTGCC | 1682 |
| chr18 | 58335371 | 58335388 | +/− | 18 | ATCTCACGTCACCTGCCT | 1683 |
| chr18 | 58335372 | 58335389 | +/− | 18 | TCTCACGTCACCTGCCTT | 1684 |
| chr18 | 58335373 | 58335390 | +/− | 18 | CTCACGTCACCTGCCTTA | 1685 |
| chr18 | 58335374 | 58335391 | +/− | 18 | TCACGTCACCTGCCTTAC | 1686 |
| chr18 | 58335375 | 58335392 | +/− | 18 | CACGTCACCTGCCTTACA | 1687 |
| chr18 | 58335376 | 58335393 | +/− | 18 | ACGTCACCTGCCTTACAG | 1688 |
| chr18 | 58335377 | 58335394 | +/− | 18 | CGTCACCTGCCTTACAGC | 1689 |
| chr18 | 58335378 | 58335395 | +/− | 18 | GTCACCTGCCTTACAGCG | 1690 |
| chr18 | 58335379 | 58335396 | +/− | 18 | TCACCTGCCTTACAGCGC | 1691 |
| chr18 | 58335380 | 58335397 | +/− | 18 | CACCTGCCTTACAGCGCT | 1692 |
| chr18 | 58335381 | 58335398 | +/− | 18 | ACCTGCCTTACAGCGCTG | 1693 |
| chr18 | 58335382 | 58335399 | +/− | 18 | CCTGCCTTACAGCGCTGC | 1694 |
| chr18 | 58335383 | 58335400 | +/− | 18 | CTGCCTTACAGCGCTGCC | 1695 |
| chr18 | 58335384 | 58335401 | +/− | 18 | TGCCTTACAGCGCTGCCA | 1696 |
| chr18 | 58335385 | 58335402 | +/− | 18 | GCCTTACAGCGCTGCCAC | 1697 |
| chr18 | 58335386 | 58335403 | +/− | 18 | CCTTACAGCGCTGCCACA | 1698 |
| chr18 | 58335387 | 58335404 | +/− | 18 | CTTACAGCGCTGCCACAG | 1699 |
| chr18 | 58335388 | 58335405 | +/− | 18 | TTACAGCGCTGCCACAGC | 1700 |
| chr18 | 58335389 | 58335406 | +/− | 18 | TACAGCGCTGCCACAGCA | 1701 |
| chr18 | 58335390 | 58335407 | +/− | 18 | ACAGCGCTGCCACAGCAG | 1702 |
| chr18 | 58335391 | 58335408 | +/− | 18 | CAGCGCTGCCACAGCAGT | 1703 |
| chr18 | 58335392 | 58335409 | +/− | 18 | AGCGCTGCCACAGCAGTG | 1704 |
| chr18 | 58335393 | 58335410 | +/− | 18 | GCGCTGCCACAGCAGTGG | 1705 |
| chr18 | 58335394 | 58335411 | +/− | 18 | CGCTGCCACAGCAGTGGG | 1706 |
| chr18 | 58335395 | 58335412 | +/− | 18 | GCTGCCACAGCAGTGGGC | 1707 |
| chr18 | 58335396 | 58335413 | +/− | 18 | CTGCCACAGCAGTGGGCC | 1708 |
| chr18 | 58335397 | 58335414 | +/− | 18 | TGCCACAGCAGTGGGCCC | 1709 |
| chr18 | 58335398 | 58335415 | +/− | 18 | GCCACAGCAGTGGGCCCT | 1710 |
| chr18 | 58335399 | 58335416 | +/− | 18 | CCACAGCAGTGGGCCCTG | 1711 |
| chr18 | 58335400 | 58335417 | +/− | 18 | CACAGCAGTGGGCCCTGA | 1712 |
| chr18 | 58335401 | 58335418 | +/− | 18 | ACAGCAGTGGGCCCTGAT | 1713 |
| chr18 | 58335402 | 58335419 | +/− | 18 | CAGCAGTGGGCCCTGATT | 1714 |
| chr18 | 58335403 | 58335420 | +/− | 18 | AGCAGTGGGCCCTGATTC | 1715 |
| chr18 | 58335404 | 58335421 | +/− | 18 | GCAGTGGGCCCTGATTCA | 1716 |
| chr18 | 58335405 | 58335422 | +/− | 18 | CAGTGGGCCCTGATTCAG | 1717 |
| chr18 | 58335406 | 58335423 | +/− | 18 | AGTGGGCCCTGATTCAGA | 1718 |
| chr18 | 58335407 | 58335424 | +/− | 18 | GTGGGCCCTGATTCAGAC | 1719 |
| chr18 | 58335408 | 58335425 | +/− | 18 | TGGGCCCTGATTCAGACA | 1720 |
| chr18 | 58335409 | 58335426 | +/− | 18 | GGGCCCTGATTCAGACAG | 1721 |
| chr18 | 58335410 | 58335427 | +/− | 18 | GGCCCTGATTCAGACAGC | 1722 |
| chr18 | 58335411 | 58335428 | +/− | 18 | GCCCTGATTCAGACAGCA | 1723 |
| chr18 | 58335412 | 58335429 | +/− | 18 | CCCTGATTCAGACAGCAG | 1724 |
| chr18 | 58335413 | 58335430 | +/− | 18 | CCTGATTCAGACAGCAGG | 1725 |
| chr18 | 58335317 | 58335334 | +/− | 18 | TGGTCATCAGCGACTGCT | 1726 |
| chr18 | 58335414 | 58335431 | +/− | 18 | CTGATTCAGACAGCAGGG | 1727 |
| chr18 | 58335415 | 58335432 | +/− | 18 | TGATTCAGACAGCAGGGG | 1728 |
| chr18 | 58335416 | 58335433 | +/− | 18 | GATTCAGACAGCAGGGGG | 1729 |
| chr18 | 58335417 | 58335434 | +/− | 18 | ATTCAGACAGCAGGGGGT | 1730 |
| chr18 | 58335418 | 58335435 | +/− | 18 | TTCAGACAGCAGGGGGTC | 1731 |
| chr18 | 58335419 | 58335436 | +/− | 18 | TCAGACAGCAGGGGGTCA | 1732 |
| chr18 | 58335420 | 58335437 | +/− | 18 | CAGACAGCAGGGGGTCAT | 1733 |
| chr18 | 58335421 | 58335438 | +/− | 18 | AGACAGCAGGGGGTCATC | 1734 |
| chr18 | 58335422 | 58335439 | +/− | 18 | GACAGCAGGGGGTCATCC | 1735 |
| chr18 | 58335423 | 58335440 | +/− | 18 | ACAGCAGGGGGTCATCCC | 1736 |
| chr18 | 58335424 | 58335441 | +/− | 18 | CAGCAGGGGGTCATCCCC | 1737 |
| chr18 | 58335425 | 58335442 | +/− | 18 | AGCAGGGGGTCATCCCCT | 1738 |
| chr18 | 58335426 | 58335443 | +/− | 18 | GCAGGGGGTCATCCCCTA | 1739 |
| chr18 | 58335427 | 58335444 | +/− | 18 | CAGGGGGTCATCCCCTAA | 1740 |
| chr18 | 58335428 | 58335445 | +/− | 18 | AGGGGGTCATCCCCTAAG | 1741 |
| chr18 | 58335429 | 58335446 | +/− | 18 | GGGGGTCATCCCCTAAGT | 1742 |
| chr18 | 58335430 | 58335447 | +/− | 18 | GGGGTCATCCCCTAAGTG | 1743 |

TABLE 12
19-mer target-specific ASOs

| | | | | | | SEQ |
|---|---|---|---|---|---|---|
| | | | | | | ID |
| CHR | START | END | STRAND | Kmer | SEQUENCE | NO: |
| chr18 | 58335318 | 58335336 | +/− | 19 | GGTCATCAGCGACTGCTGG | 1744 |
| chr18 | 58335319 | 58335337 | +/− | 19 | GTCATCAGCGACTGCTGGC | 1745 |
| chr18 | 58335320 | 58335338 | +/− | 19 | TCATCAGCGACTGCTGGCT | 1746 |
| chr18 | 58335321 | 58335339 | +/− | 19 | CATCAGCGACTGCTGGCTT | 1747 |
| chr18 | 58335322 | 58335340 | +/− | 19 | ATCAGCGACTGCTGGCTTT | 1748 |
| chr18 | 58335323 | 58335341 | +/− | 19 | TCAGCGACTGCTGGCTTTG | 1749 |
| chr18 | 58335324 | 58335342 | +/− | 19 | CAGCGACTGCTGGCTTTGT | 1750 |
| chr18 | 58335325 | 58335343 | +/− | 19 | AGCGACTGCTGGCTTTGTC | 1751 |
| chr18 | 58335326 | 58335344 | +/− | 19 | GCGACTGCTGGCTTTGTCT | 1752 |
| chr18 | 58335327 | 58335345 | +/− | 19 | CGACTGCTGGCTTTGTCTG | 1753 |
| chr18 | 58335328 | 58335346 | +/− | 19 | GACTGCTGGCTTTGTCTGG | 1754 |
| chr18 | 58335329 | 58335347 | +/− | 19 | ACTGCTGGCTTTGTCTGGA | 1755 |
| chr18 | 58335330 | 58335348 | +/− | 19 | CTGCTGGCTTTGTCTGGAT | 1756 |
| chr18 | 58335331 | 58335349 | +/− | 19 | TGCTGGCTTTGTCTGGATA | 1757 |
| chr18 | 58335332 | 58335350 | +/− | 19 | GCTGGCTTTGTCTGGATAG | 1758 |
| chr18 | 58335333 | 58335351 | +/− | 19 | CTGGCTTTGTCTGGATAGG | 1759 |
| chr18 | 58335334 | 58335352 | +/− | 19 | TGGCTTTGTCTGGATAGGG | 1760 |
| chr18 | 58335335 | 58335353 | +/− | 19 | GGCTTTGTCTGGATAGGGT | 1761 |
| chr18 | 58335336 | 58335354 | +/− | 19 | GCTTTGTCTGGATAGGGTG | 1762 |
| chr18 | 58335337 | 58335355 | +/− | 19 | CTTTGTCTGGATAGGGTGG | 1763 |
| chr18 | 58335338 | 58335356 | +/− | 19 | TTTGTCTGGATAGGGTGGG | 1764 |
| chr18 | 58335339 | 58335357 | +/− | 19 | TTGTCTGGATAGGGTGGGT | 1765 |
| chr18 | 58335340 | 58335358 | +/− | 19 | TGTCTGGATAGGGTGGGTT | 1766 |
| chr18 | 58335341 | 58335359 | +/− | 19 | GTCTGGATAGGGTGGGTTT | 1767 |
| chr18 | 58335342 | 58335360 | +/− | 19 | TCTGGATAGGGTGGGTTTC | 1768 |
| chr18 | 58335343 | 58335361 | +/− | 19 | CTGGATAGGGTGGGTTTCA | 1769 |
| chr18 | 58335344 | 58335362 | +/− | 19 | TGGATAGGGTGGGTTTCAG | 1770 |
| chr18 | 58335345 | 58335363 | +/− | 19 | GGATAGGGTGGGTTTCAGG | 1771 |
| chr18 | 58335346 | 58335364 | +/− | 19 | GATAGGGTGGGTTTCAGGG | 1772 |
| chr18 | 58335347 | 58335365 | +/− | 19 | ATAGGGTGGGTTTCAGGGA | 1773 |
| chr18 | 58335348 | 58335366 | +/− | 19 | TAGGGTGGGTTTCAGGGAT | 1774 |
| chr18 | 58335349 | 58335367 | +/− | 19 | AGGGTGGGTTTCAGGGATT | 1775 |
| chr18 | 58335350 | 58335368 | +/− | 19 | GGGTGGGTTTCAGGGATTC | 1776 |
| chr18 | 58335351 | 58335369 | +/− | 19 | GGTGGGTTTCAGGGATTCT | 1777 |
| chr18 | 58335352 | 58335370 | +/− | 19 | GTGGGTTTCAGGGATTCTG | 1778 |
| chr18 | 58335353 | 58335371 | +/− | 19 | TGGGTTTCAGGGATTCTGA | 1779 |
| chr18 | 58335354 | 58335372 | +/− | 19 | GGGTTTCAGGGATTCTGAT | 1780 |
| chr18 | 58335355 | 58335373 | +/− | 19 | GGTTTCAGGGATTCTGATC | 1781 |
| chr18 | 58335356 | 58335374 | +/− | 19 | GTTTCAGGGATTCTGATCT | 1782 |
| chr18 | 58335357 | 58335375 | +/− | 19 | TTTCAGGGATTCTGATCTC | 1783 |
| chr18 | 58335358 | 58335376 | +/− | 19 | TTCAGGGATTCTGATCTCA | 1784 |
| chr18 | 58335359 | 58335377 | +/− | 19 | TCAGGGATTCTGATCTCAC | 1785 |
| chr18 | 58335360 | 58335378 | +/− | 19 | CAGGGATTCTGATCTCACG | 1786 |
| chr18 | 58335361 | 58335379 | +/− | 19 | AGGGATTCTGATCTCACGT | 1787 |
| chr18 | 58335362 | 58335380 | +/− | 19 | GGGATTCTGATCTCACGTC | 1788 |
| chr18 | 58335363 | 58335381 | +/− | 19 | GGATTCTGATCTCACGTCA | 1789 |
| chr18 | 58335364 | 58335382 | +/− | 19 | GATTCTGATCTCACGTCAC | 1790 |
| chr18 | 58335365 | 58335383 | +/− | 19 | ATTCTGATCTCACGTCACC | 1791 |
| chr18 | 58335366 | 58335384 | +/− | 19 | TTCTGATCTCACGTCACCT | 1792 |
| chr18 | 58335367 | 58335385 | +/− | 19 | TCTGATCTCACGTCACCTG | 1793 |
| chr18 | 58335368 | 58335386 | +/− | 19 | CTGATCTCACGTCACCTGC | 1794 |
| chr18 | 58335369 | 58335387 | +/− | 19 | TGATCTCACGTCACCTGCC | 1795 |
| chr18 | 58335370 | 58335388 | +/− | 19 | GATCTCACGTCACCTGCCT | 1796 |
| chr18 | 58335371 | 58335389 | +/− | 19 | ATCTCACGTCACCTGCCTT | 1797 |
| chr18 | 58335372 | 58335390 | +/− | 19 | TCTCACGTCACCTGCCTTA | 1798 |
| chr18 | 58335373 | 58335391 | +/− | 19 | CTCACGTCACCTGCCTTAC | 1799 |
| chr18 | 58335374 | 58335392 | +/− | 19 | TCACGTCACCTGCCTTACA | 1800 |
| chr18 | 58335375 | 58335393 | +/− | 19 | CACGTCACCTGCCTTACAG | 1801 |
| chr18 | 58335376 | 58335394 | +/− | 19 | ACGTCACCTGCCTTACAGC | 1802 |
| chr18 | 58335377 | 58335395 | +/− | 19 | CGTCACCTGCCTTACAGCG | 1803 |
| chr18 | 58335378 | 58335396 | +/− | 19 | GTCACCTGCCTTACAGCGC | 1804 |
| chr18 | 58335379 | 58335397 | +/− | 19 | TCACCTGCCTTACAGCGCT | 1805 |
| chr18 | 58335380 | 58335398 | +/− | 19 | CACCTGCCTTACAGCGCTG | 1806 |
| chr18 | 58335381 | 58335399 | +/− | 19 | ACCTGCCTTACAGCGCTGC | 1807 |
| chr18 | 58335382 | 58335400 | +/− | 19 | CCTGCCTTACAGCGCTGCC | 1808 |
| chr18 | 58335383 | 58335401 | +/− | 19 | CTGCCTTACAGCGCTGCCA | 1809 |
| chr18 | 58335384 | 58335402 | +/− | 19 | TGCCTTACAGCGCTGCCAC | 1810 |
| chr18 | 58335385 | 58335403 | +/− | 19 | GCCTTACAGCGCTGCCACA | 1811 |
| chr18 | 58335386 | 58335404 | +/− | 19 | CCTTACAGCGCTGCCACAG | 1812 |
| chr18 | 58335387 | 58335405 | +/− | 19 | CTTACAGCGCTGCCACAGC | 1813 |
| chr18 | 58335388 | 58335406 | +/− | 19 | TTACAGCGCTGCCACAGCA | 1814 |
| chr18 | 58335389 | 58335407 | +/− | 19 | TACAGCGCTGCCACAGCAG | 1815 |
| chr18 | 58335390 | 58335408 | +/− | 19 | ACAGCGCTGCCACAGCAGT | 1816 |
| chr18 | 58335391 | 58335409 | +/− | 19 | CAGCGCTGCCACAGCAGTG | 1817 |
| chr18 | 58335392 | 58335410 | +/− | 19 | AGCGCTGCCACAGCAGTGG | 1818 |
| chr18 | 58335393 | 58335411 | +/− | 19 | GCGCTGCCACAGCAGTGGG | 1819 |
| chr18 | 58335394 | 58335412 | +/− | 19 | CGCTGCCACAGCAGTGGGC | 1820 |
| chr18 | 58335395 | 58335413 | +/− | 19 | GCTGCCACAGCAGTGGGCC | 1821 |
| chr18 | 58335396 | 58335414 | +/− | 19 | CTGCCACAGCAGTGGGCCC | 1822 |
| chr18 | 58335397 | 58335415 | +/− | 19 | TGCCACAGCAGTGGGCCCT | 1823 |
| chr18 | 58335398 | 58335416 | +/− | 19 | GCCACAGCAGTGGGCCCTG | 1824 |
| chr18 | 58335399 | 58335417 | +/− | 19 | CCACAGCAGTGGGCCCTGA | 1825 |
| chr18 | 58335400 | 58335418 | +/− | 19 | CACAGCAGTGGGCCCTGAT | 1826 |
| chr18 | 58335401 | 58335419 | +/− | 19 | ACAGCAGTGGGCCCTGATT | 1827 |
| chr18 | 58335402 | 58335420 | +/− | 19 | CAGCAGTGGGCCCTGATTC | 1828 |
| chr18 | 58335403 | 58335421 | +/− | 19 | AGCAGTGGGCCCTGATTCA | 1829 |
| chr18 | 58335404 | 58335422 | +/− | 19 | GCAGTGGGCCCTGATTCAG | 1830 |
| chr18 | 58335405 | 58335423 | +/− | 19 | CAGTGGGCCCTGATTCAGA | 1831 |
| chr18 | 58335406 | 58335424 | +/− | 19 | AGTGGGCCCTGATTCAGAC | 1832 |
| chr18 | 58335407 | 58335425 | +/− | 19 | GTGGGCCCTGATTCAGACA | 1833 |
| chr18 | 58335408 | 58335426 | +/− | 19 | TGGGCCCTGATTCAGACAG | 1834 |
| chr18 | 58335409 | 58335427 | +/− | 19 | GGGCCCTGATTCAGACAGC | 1835 |
| chr18 | 58335410 | 58335428 | +/− | 19 | GGCCCTGATTCAGACAGCA | 1836 |
| chr18 | 58335411 | 58335429 | +/− | 19 | GCCCTGATTCAGACAGCAG | 1837 |
| chr18 | 58335412 | 58335430 | +/− | 19 | CCCTGATTCAGACAGCAGG | 1838 |
| chr18 | 58335317 | 58335335 | +/− | 19 | TGGTCATCAGCGACTGCTG | 1839 |
| chr18 | 58335413 | 58335431 | +/− | 19 | CCTGATTCAGACAGCAGGG | 1840 |
| chr18 | 58335414 | 58335432 | +/− | 19 | CTGATTCAGACAGCAGGGG | 1841 |
| chr18 | 58335415 | 58335433 | +/− | 19 | TGATTCAGACAGCAGGGGG | 1842 |
| chr18 | 58335416 | 58335434 | +/− | 19 | GATTCAGACAGCAGGGGGT | 1843 |
| chr18 | 58335417 | 58335435 | +/− | 19 | ATTCAGACAGCAGGGGGTC | 1844 |
| chr18 | 58335418 | 58335436 | +/− | 19 | TTCAGACAGCAGGGGGTCA | 1845 |
| chr18 | 58335419 | 58335437 | +/− | 19 | TCAGACAGCAGGGGGTCAT | 1846 |
| chr18 | 58335420 | 58335438 | +/− | 19 | CAGACAGCAGGGGGTCATC | 1847 |
| chr18 | 58335421 | 58335439 | +/− | 19 | AGACAGCAGGGGGTCATCC | 1848 |
| chr18 | 58335422 | 58335440 | +/− | 19 | GACAGCAGGGGGTCATCCC | 1849 |
| chr18 | 58335423 | 58335441 | +/− | 19 | ACAGCAGGGGGTCATCCCC | 1850 |
| chr18 | 58335424 | 58335442 | +/− | 19 | CAGCAGGGGGTCATCCCCT | 1851 |
| chr18 | 58335425 | 58335443 | +/− | 19 | AGCAGGGGGTCATCCCCTA | 1852 |
| chr18 | 58335426 | 58335444 | +/− | 19 | GCAGGGGGTCATCCCCTAA | 1853 |
| chr18 | 58335427 | 58335445 | +/− | 19 | CAGGGGGTCATCCCCTAAG | 1854 |
| chr18 | 58335428 | 58335446 | +/− | 19 | AGGGGGTCATCCCCTAAGT | 1855 |
| chr18 | 58335429 | 58335447 | +/− | 19 | GGGGGTCATCCCCTAAGTG | 1856 |

TABLE 13
20-mer target-specific ASOs

| | | | | | | SEQ |
|---|---|---|---|---|---|---|
| | | | | | | ID |
| CHR | START | END | STRAND | Kmer | SEQUENCE | NO: |
| chr18 | 58335318 | 58335337 | +/− | 20 | GGTCATCAGCGACTGCTGGC | 1857 |
| chr18 | 58335319 | 58335338 | +/− | 20 | GTCATCAGCGACTGCTGGCT | 1858 |
| chr18 | 58335320 | 58335339 | +/− | 20 | TCATCAGCGACTGCTGGCTT | 1859 |
| chr18 | 58335321 | 58335340 | +/− | 20 | CATCAGCGACTGCTGGCTTT | 1860 |
| chr18 | 58335322 | 58335341 | +/− | 20 | ATCAGCGACTGCTGGCTTTG | 1861 |
| chr18 | 58335323 | 58335342 | +/− | 20 | TCAGCGACTGCTGGCTTTGT | 1862 |
| chr18 | 58335324 | 58335343 | +/− | 20 | CAGCGACTGCTGGCTTTGTC | 1863 |
| chr18 | 58335325 | 58335344 | +/− | 20 | AGCGACTGCTGGCTTTGTCT | 1864 |
| chr18 | 58335326 | 58335345 | +/− | 20 | GCGACTGCTGGCTTTGTCTG | 1865 |
| chr18 | 58335327 | 58335346 | +/− | 20 | CGACTGCTGGCTTTGTCTGG | 1866 |
| chr18 | 58335328 | 58335347 | +/− | 20 | GACTGCTGGCTTTGTCTGGA | 1867 |
| chr18 | 58335329 | 58335348 | +/− | 20 | ACTGCTGGCTTTGTCTGGAT | 1868 |
| chr18 | 58335330 | 58335349 | +/− | 20 | CTGCTGGCTTTGTCTGGATA | 1869 |
| chr18 | 58335331 | 58335350 | +/− | 20 | TGCTGGCTTTGTCTGGATAG | 1870 |
| chr18 | 58335332 | 58335351 | +/− | 20 | GCTGGCTTTGTCTGGATAGG | 3 |
| chr18 | 58335333 | 58335352 | +/− | 20 | CTGGCTTTGTCTGGATAGGG | 1871 |
| chr18 | 58335334 | 58335353 | +/− | 20 | TGGCTTTGTCTGGATAGGGT | 1872 |
| chr18 | 58335335 | 58335354 | +/− | 20 | GGCTTTGTCTGGATAGGGTG | 1873 |
| chr18 | 58335336 | 58335355 | +/− | 20 | GCTTTGTCTGGATAGGGTGG | 1874 |
| chr18 | 58335337 | 58335356 | +/− | 20 | CTTTGTCTGGATAGGGTGGG | 1875 |
| chr18 | 58335338 | 58335357 | +/− | 20 | TTTGTCTGGATAGGGTGGGT | 1876 |
| chr18 | 58335339 | 58335358 | +/− | 20 | TTGTCTGGATAGGGTGGGTT | 1877 |
| chr18 | 58335340 | 58335359 | +/− | 20 | TGTCTGGATAGGGTGGGTTT | 1878 |
| chr18 | 58335341 | 58335360 | +/− | 20 | GTCTGGATAGGGTGGGTTTC | 1879 |
| chr18 | 58335342 | 58335361 | +/− | 20 | TCTGGATAGGGTGGGTTTCA | 1880 |
| chr18 | 58335343 | 58335362 | +/− | 20 | CTGGATAGGGTGGGTTTCAG | 1881 |
| chr18 | 58335344 | 58335363 | +/− | 20 | TGGATAGGGTGGGTTTCAGG | 1882 |
| chr18 | 58335345 | 58335364 | +/− | 20 | GGATAGGGTGGGTTTCAGGG | 1883 |
| chr18 | 58335346 | 58335365 | +/− | 20 | GATAGGGTGGGTTTCAGGGA | 1884 |
| chr18 | 58335347 | 58335366 | +/− | 20 | ATAGGGTGGGTTTCAGGGAT | 1885 |
| chr18 | 58335348 | 58335367 | +/− | 20 | TAGGGTGGGTTTCAGGGATT | 1886 |
| chr18 | 58335349 | 58335368 | +/− | 20 | AGGGTGGGTTTCAGGGATTC | 1887 |
| chr18 | 58335350 | 58335369 | +/− | 20 | GGGTGGGTTTCAGGGATTCT | 1888 |
| chr18 | 58335351 | 58335370 | +/− | 20 | GGTGGGTTTCAGGGATTCTG | 1889 |
| chr18 | 58335352 | 58335371 | +/− | 20 | GTGGGTTTCAGGGATTCTGA | 1 |
| chr18 | 58335353 | 58335372 | +/− | 20 | TGGGTTTCAGGGATTCTGAT | 1890 |
| chr18 | 58335354 | 58335373 | +/− | 20 | GGGTTTCAGGGATTCTGATC | 1891 |
| chr18 | 58335355 | 58335374 | +/− | 20 | GGTTTCAGGGATTCTGATCT | 1892 |
| chr18 | 58335356 | 58335375 | +/− | 20 | GTTTCAGGGATTCTGATCTC | 1893 |
| chr18 | 58335357 | 58335376 | +/− | 20 | TTTCAGGGATTCTGATCTCA | 1894 |
| chr18 | 58335358 | 58335377 | +/− | 20 | TTCAGGGATTCTGATCTCAC | 1895 |
| chr18 | 58335359 | 58335378 | +/− | 20 | TCAGGGATTCTGATCTCACG | 1896 |
| chr18 | 58335360 | 58335379 | +/− | 20 | CAGGGATTCTGATCTCACGT | 1897 |
| chr18 | 58335361 | 58335380 | +/− | 20 | AGGGATTCTGATCTCACGTC | 1898 |
| chr18 | 58335362 | 58335381 | +/− | 20 | GGGATTCTGATCTCACGTCA | 1899 |
| chr18 | 58335363 | 58335382 | +/− | 20 | GGATTCTGATCTCACGTCAC | 1900 |
| chr18 | 58335364 | 58335383 | +/− | 20 | GATTCTGATCTCACGTCACC | 1901 |
| chr18 | 58335365 | 58335384 | +/− | 20 | ATTCTGATCTCACGTCACCT | 1902 |
| chr18 | 58335366 | 58335385 | +/− | 20 | TTCTGATCTCACGTCACCTG | 1903 |
| chr18 | 58335367 | 58335386 | +/− | 20 | TCTGATCTCACGTCACCTGC | 1904 |
| chr18 | 58335368 | 58335387 | +/− | 20 | CTGATCTCACGTCACCTGCC | 1905 |
| chr18 | 58335369 | 58335388 | +/− | 20 | TGATCTCACGTCACCTGCCT | 1906 |
| chr18 | 58335370 | 58335389 | +/− | 20 | GATCTCACGTCACCTGCCTT | 1907 |
| chr18 | 58335371 | 58335390 | +/− | 20 | ATCTCACGTCACCTGCCTTA | 1908 |
| chr18 | 58335372 | 58335391 | +/− | 20 | TCTCACGTCACCTGCCTTAC | 4 |
| chr18 | 58335373 | 58335392 | +/− | 20 | CTCACGTCACCTGCCTTACA | 1909 |
| chr18 | 58335374 | 58335393 | +/− | 20 | TCACGTCACCTGCCTTACAG | 1910 |
| chr18 | 58335375 | 58335394 | +/− | 20 | CACGTCACCTGCCTTACAGC | 1911 |
| chr18 | 58335376 | 58335395 | +/− | 20 | ACGTCACCTGCCTTACAGCG | 1912 |
| chr18 | 58335377 | 58335396 | +/− | 20 | CGTCACCTGCCTTACAGCGC | 1913 |
| chr18 | 58335378 | 58335397 | +/− | 20 | GTCACCTGCCTTACAGCGCT | 1914 |
| chr18 | 58335379 | 58335398 | +/− | 20 | TCACCTGCCTTACAGCGCTG | 1915 |
| chr18 | 58335380 | 58335399 | +/− | 20 | CACCTGCCTTACAGCGCTGC | 1916 |
| chr18 | 58335381 | 58335400 | +/− | 20 | ACCTGCCTTACAGCGCTGCC | 1917 |
| chr18 | 58335382 | 58335401 | +/− | 20 | CCTGCCTTACAGCGCTGCCA | 1918 |
| chr18 | 58335383 | 58335402 | +/− | 20 | CTGCCTTACAGCGCTGCCAC | 1919 |
| chr18 | 58335384 | 58335403 | +/− | 20 | TGCCTTACAGCGCTGCCACA | 1920 |
| chr18 | 58335385 | 58335404 | +/− | 20 | GCCTTACAGCGCTGCCACAG | 1921 |
| chr18 | 58335386 | 58335405 | +/− | 20 | CCTTACAGCGCTGCCACAGC | 1922 |
| chr18 | 58335387 | 58335406 | +/− | 20 | CTTACAGCGCTGCCACAGCA | 1923 |
| chr18 | 58335388 | 58335407 | +/− | 20 | TTACAGCGCTGCCACAGCAG | 1924 |
| chr18 | 58335389 | 58335408 | +/− | 20 | TACAGCGCTGCCACAGCAGT | 1925 |
| chr18 | 58335390 | 58335409 | +/− | 20 | ACAGCGCTGCCACAGCAGTG | 1926 |
| chr18 | 58335391 | 58335410 | +/− | 20 | CAGCGCTGCCACAGCAGTGG | 1927 |
| chr18 | 58335392 | 58335411 | +/− | 20 | AGCGCTGCCACAGCAGTGGG | 5 |
| chr18 | 58335393 | 58335412 | +/− | 20 | GCGCTGCCACAGCAGTGGGC | 1928 |
| chr18 | 58335394 | 58335413 | +/− | 20 | CGCTGCCACAGCAGTGGGCC | 1929 |
| chr18 | 58335395 | 58335414 | +/− | 20 | GCTGCCACAGCAGTGGGCCC | 1930 |
| chr18 | 58335396 | 58335415 | +/− | 20 | CTGCCACAGCAGTGGGCCCT | 1931 |
| chr18 | 58335397 | 58335416 | +/− | 20 | TGCCACAGCAGTGGGCCCTG | 1932 |
| chr18 | 58335398 | 58335417 | +/− | 20 | GCCACAGCAGTGGGCCCTGA | 1933 |
| chr18 | 58335399 | 58335418 | +/− | 20 | CCACAGCAGTGGGCCCTGAT | 1934 |
| chr18 | 58335400 | 58335419 | +/− | 20 | CACAGCAGTGGGCCCTGATT | 1935 |
| chr18 | 58335401 | 58335420 | +/− | 20 | ACAGCAGTGGGCCCTGATTC | 1936 |
| chr18 | 58335402 | 58335421 | +/− | 20 | CAGCAGTGGGCCCTGATTCA | 1937 |
| chr18 | 58335403 | 58335422 | +/− | 20 | AGCAGTGGGCCCTGATTCAG | 1938 |
| chr18 | 58335404 | 58335423 | +/− | 20 | GCAGTGGGCCCTGATTCAGA | 1939 |
| chr18 | 58335405 | 58335424 | +/− | 20 | CAGTGGGCCCTGATTCAGAC | 1940 |
| chr18 | 58335406 | 58335425 | +/− | 20 | AGTGGGCCCTGATTCAGACA | 1941 |
| chr18 | 58335407 | 58335426 | +/− | 20 | GTGGGCCCTGATTCAGACAG | 1942 |
| chr18 | 58335408 | 58335427 | +/− | 20 | TGGGCCCTGATTCAGACAGC | 1943 |
| chr18 | 58335409 | 58335428 | +/− | 20 | GGGCCCTGATTCAGACAGCA | 1944 |
| chr18 | 58335410 | 58335429 | +/− | 20 | GGCCCTGATTCAGACAGCAG | 1945 |
| chr18 | 58335411 | 58335430 | +/− | 20 | GCCCTGATTCAGACAGCAGG | 1946 |
| chr18 | 58335317 | 58335336 | +/− | 20 | TGGTCATCAGCGACTGCTGG | 1947 |
| chr18 | 58335412 | 58335431 | +/− | 20 | CCCTGATTCAGACAGCAGGG | 2 |
| chr18 | 58335413 | 58335432 | +/− | 20 | CCTGATTCAGACAGCAGGGG | 1948 |
| chr18 | 58335414 | 58335433 | +/− | 20 | CTGATTCAGACAGCAGGGGG | 1949 |
| chr18 | 58335415 | 58335434 | +/− | 20 | TGATTCAGACAGCAGGGGGT | 1950 |
| chr18 | 58335416 | 58335435 | +/− | 20 | GATTCAGACAGCAGGGGGTC | 1951 |
| chr18 | 58335417 | 58335436 | +/− | 20 | ATTCAGACAGCAGGGGGTCA | 1952 |
| chr18 | 58335418 | 58335437 | +/− | 20 | TTCAGACAGCAGGGGGTCAT | 1953 |
| chr18 | 58335419 | 58335438 | +/− | 20 | TCAGACAGCAGGGGGTCATC | 1954 |
| chr18 | 58335420 | 58335439 | +/− | 20 | CAGACAGCAGGGGGTCATCC | 1955 |
| chr18 | 58335421 | 58335440 | +/− | 20 | AGACAGCAGGGGGTCATCCC | 1956 |
| chr18 | 58335422 | 58335441 | +/− | 20 | GACAGCAGGGGGTCATCCCC | 1957 |
| chr18 | 58335423 | 58335442 | +/− | 20 | ACAGCAGGGGGTCATCCCCT | 1958 |
| chr18 | 58335424 | 58335443 | +/− | 20 | CAGCAGGGGGTCATCCCCTA | 1959 |
| chr18 | 58335425 | 58335444 | +/− | 20 | AGCAGGGGGTCATCCCCTAA | 1960 |
| chr18 | 58335426 | 58335445 | +/− | 20 | GCAGGGGGTCATCCCCTAAG | 1961 |
| chr18 | 58335427 | 58335446 | +/− | 20 | CAGGGGGTCATCCCCTAAGT | 1962 |
| chr18 | 58335428 | 58335447 | +/− | 20 | AGGGGGTCATCCCCTAAGTG | 1963 |

TABLE 14
21-mer target-specific ASOs

| | | | | | | SEQ |
|---|---|---|---|---|---|---|
| | | | | | | ID |
| CHR | START | END | STRAND | Kmer | SEQUENCE | NO: |
| chr18 | 58335318 | 58335338 | +/− | 21 | GGTCATCAGCGACTGCTGGCT | 1964 |
| chr18 | 58335319 | 58335339 | +/− | 21 | GTCATCAGCGACTGCTGGCTT | 1965 |
| chr18 | 58335320 | 58335340 | +/− | 21 | TCATCAGCGACTGCTGGCTTT | 1966 |
| chr18 | 58335321 | 58335341 | +/− | 21 | CATCAGCGACTGCTGGCTTTG | 1967 |
| chr18 | 58335322 | 58335342 | +/− | 21 | ATCAGCGACTGCTGGCTTTGT | 1968 |
| chr18 | 58335323 | 58335343 | +/− | 21 | TCAGCGACTGCTGGCTTTGTC | 1969 |
| chr18 | 58335324 | 58335344 | +/− | 21 | CAGCGACTGCTGGCTTTGTCT | 1970 |
| chr18 | 58335325 | 58335345 | +/− | 21 | AGCGACTGCTGGCTTTGTCTG | 1971 |
| chr18 | 58335326 | 58335346 | +/− | 21 | GCGACTGCTGGCTTTGTCTGG | 1972 |
| chr18 | 58335327 | 58335347 | +/− | 21 | CGACTGCTGGCTTTGTCTGGA | 1973 |
| chr18 | 58335328 | 58335348 | +/− | 21 | GACTGCTGGCTTTGTCTGGAT | 1974 |
| chr18 | 58335329 | 58335349 | +/− | 21 | ACTGCTGGCTTTGTCTGGATA | 1975 |
| chr18 | 58335330 | 58335350 | +/− | 21 | CTGCTGGCTTTGTCTGGATAG | 1976 |
| chr18 | 58335331 | 58335351 | +/− | 21 | TGCTGGCTTTGTCTGGATAGG | 1977 |
| chr18 | 58335332 | 58335352 | +/− | 21 | GCTGGCTTTGTCTGGATAGGG | 1978 |
| chr18 | 58335333 | 58335353 | +/− | 21 | CTGGCTTTGTCTGGATAGGGT | 1979 |
| chr18 | 58335334 | 58335354 | +/− | 21 | TGGCTTTGTCTGGATAGGGTG | 1980 |
| chr18 | 58335335 | 58335355 | +/− | 21 | GGCTTTGTCTGGATAGGGTGG | 1981 |
| chr18 | 58335336 | 58335356 | +/− | 21 | GCTTTGTCTGGATAGGGTGGG | 1982 |
| chr18 | 58335337 | 58335357 | +/− | 21 | CTTTGTCTGGATAGGGTGGGT | 1983 |
| chr18 | 58335338 | 58335358 | +/− | 21 | TTTGTCTGGATAGGGTGGGTT | 1984 |
| chr18 | 58335339 | 58335359 | +/− | 21 | TTGTCTGGATAGGGTGGGTTT | 1985 |
| chr18 | 58335340 | 58335360 | +/− | 21 | TGTCTGGATAGGGTGGGTTTC | 1986 |
| chr18 | 58335341 | 58335361 | +/− | 21 | GTCTGGATAGGGTGGGTTTCA | 1987 |
| chr18 | 58335342 | 58335362 | +/− | 21 | TCTGGATAGGGTGGGTTTCAG | 1988 |
| chr18 | 58335343 | 58335363 | +/− | 21 | CTGGATAGGGTGGGTTTCAGG | 1989 |
| chr18 | 58335344 | 58335364 | +/− | 21 | TGGATAGGGTGGGTTTCAGGG | 1990 |
| chr18 | 58335345 | 58335365 | +/− | 21 | GGATAGGGTGGGTTTCAGGGA | 1991 |
| chr18 | 58335346 | 58335366 | +/− | 21 | GATAGGGTGGGTTTCAGGGAT | 1992 |
| chr18 | 58335347 | 58335367 | +/− | 21 | ATAGGGTGGGTTTCAGGGATT | 1993 |
| chr18 | 58335348 | 58335368 | +/− | 21 | TAGGGTGGGTTTCAGGGATTC | 1994 |
| chr18 | 58335349 | 58335369 | +/− | 21 | AGGGTGGGTTTCAGGGATTCT | 1995 |
| chr18 | 58335350 | 58335370 | +/− | 21 | GGGTGGGTTTCAGGGATTCTG | 1996 |
| chr18 | 58335351 | 58335371 | +/− | 21 | GGTGGGTTTCAGGGATTCTGA | 1997 |
| chr18 | 58335352 | 58335372 | +/− | 21 | GTGGGTTTCAGGGATTCTGAT | 1998 |
| chr18 | 58335353 | 58335373 | +/− | 21 | TGGGTTTCAGGGATTCTGATC | 1999 |
| chr18 | 58335354 | 58335374 | +/− | 21 | GGGTTTCAGGGATTCTGATCT | 2000 |
| chr18 | 58335355 | 58335375 | +/− | 21 | GGTTTCAGGGATTCTGATCTC | 2001 |
| chr18 | 58335356 | 58335376 | +/− | 21 | GTTTCAGGGATTCTGATCTCA | 2002 |
| chr18 | 58335357 | 58335377 | +/− | 21 | TTTCAGGGATTCTGATCTCAC | 2003 |
| chr18 | 58335358 | 58335378 | +/− | 21 | TTCAGGGATTCTGATCTCACG | 2004 |
| chr18 | 58335359 | 58335379 | +/− | 21 | TCAGGGATTCTGATCTCACGT | 2005 |
| chr18 | 58335360 | 58335380 | +/− | 21 | CAGGGATTCTGATCTCACGTC | 2006 |
| chr18 | 58335361 | 58335381 | +/− | 21 | AGGGATTCTGATCTCACGTCA | 2007 |
| chr18 | 58335362 | 58335382 | +/− | 21 | GGGATTCTGATCTCACGTCAC | 2008 |
| chr18 | 58335363 | 58335383 | +/− | 21 | GGATTCTGATCTCACGTCACC | 2009 |
| chr18 | 58335364 | 58335384 | +/− | 21 | GATTCTGATCTCACGTCACCT | 2010 |
| chr18 | 58335365 | 58335385 | +/− | 21 | ATTCTGATCTCACGTCACCTG | 2011 |
| chr18 | 58335366 | 58335386 | +/− | 21 | TTCTGATCTCACGTCACCTGC | 2012 |
| chr18 | 58335367 | 58335387 | +/− | 21 | TCTGATCTCACGTCACCTGCC | 2013 |
| chr18 | 58335368 | 58335388 | +/− | 21 | CTGATCTCACGTCACCTGCCT | 2014 |
| chr18 | 58335369 | 58335389 | +/− | 21 | TGATCTCACGTCACCTGCCTT | 2015 |
| chr18 | 58335370 | 58335390 | +/− | 21 | GATCTCACGTCACCTGCCTTA | 2016 |
| chr18 | 58335371 | 58335391 | +/− | 21 | ATCTCACGTCACCTGCCTTAC | 2017 |
| chr18 | 58335372 | 58335392 | +/− | 21 | TCTCACGTCACCTGCCTTACA | 2018 |
| chr18 | 58335373 | 58335393 | +/− | 21 | CTCACGTCACCTGCCTTACAG | 2019 |
| chr18 | 58335374 | 58335394 | +/− | 21 | TCACGTCACCTGCCTTACAGC | 2020 |
| chr18 | 58335375 | 58335395 | +/− | 21 | CACGTCACCTGCCTTACAGCG | 2021 |
| chr18 | 58335376 | 58335396 | +/− | 21 | ACGTCACCTGCCTTACAGCGC | 2022 |
| chr18 | 58335377 | 58335397 | +/− | 21 | CGTCACCTGCCTTACAGCGCT | 2023 |
| chr18 | 58335378 | 58335398 | +/− | 21 | GTCACCTGCCTTACAGCGCTG | 2024 |
| chr18 | 58335379 | 58335399 | +/− | 21 | TCACCTGCCTTACAGCGCTGC | 2025 |
| chr18 | 58335380 | 58335400 | +/− | 21 | CACCTGCCTTACAGCGCTGCC | 2026 |
| chr18 | 58335381 | 58335401 | +/− | 21 | ACCTGCCTTACAGCGCTGCCA | 2027 |
| chr18 | 58335382 | 58335402 | +/− | 21 | CCTGCCTTACAGCGCTGCCAC | 2028 |
| chr18 | 58335383 | 58335403 | +/− | 21 | CTGCCTTACAGCGCTGCCACA | 2029 |
| chr18 | 58335384 | 58335404 | +/− | 21 | TGCCTTACAGCGCTGCCACAG | 2030 |
| chr18 | 58335385 | 58335405 | +/− | 21 | GCCTTACAGCGCTGCCACAGC | 2031 |
| chr18 | 58335386 | 58335406 | +/− | 21 | CCTTACAGCGCTGCCACAGCA | 2032 |
| chr18 | 58335387 | 58335407 | +/− | 21 | CTTACAGCGCTGCCACAGCAG | 2033 |
| chr18 | 58335388 | 58335408 | +/− | 21 | TTACAGCGCTGCCACAGCAGT | 2034 |
| chr18 | 58335389 | 58335409 | +/− | 21 | TACAGCGCTGCCACAGCAGTG | 2035 |
| chr18 | 58335390 | 58335410 | +/− | 21 | ACAGCGCTGCCACAGCAGTGG | 2036 |
| chr18 | 58335391 | 58335411 | +/− | 21 | CAGCGCTGCCACAGCAGTGGG | 2037 |
| chr18 | 58335392 | 58335412 | +/− | 21 | AGCGCTGCCACAGCAGTGGGC | 2038 |
| chr18 | 58335393 | 58335413 | +/− | 21 | GCGCTGCCACAGCAGTGGGCC | 2039 |
| chr18 | 58335394 | 58335414 | +/− | 21 | CGCTGCCACAGCAGTGGGCCC | 2040 |
| chr18 | 58335395 | 58335415 | +/− | 21 | GCTGCCACAGCAGTGGGCCCT | 2041 |
| chr18 | 58335396 | 58335416 | +/− | 21 | CTGCCACAGCAGTGGGCCCTG | 2042 |
| chr18 | 58335397 | 58335417 | +/− | 21 | TGCCACAGCAGTGGGCCCTGA | 2043 |
| chr18 | 58335398 | 58335418 | +/− | 21 | GCCACAGCAGTGGGCCCTGAT | 2044 |
| chr18 | 58335399 | 58335419 | +/− | 21 | CCACAGCAGTGGGCCCTGATT | 2045 |
| chr18 | 58335400 | 58335420 | +/− | 21 | CACAGCAGTGGGCCCTGATTC | 2046 |
| chr18 | 58335401 | 58335421 | +/− | 21 | ACAGCAGTGGGCCCTGATTCA | 2047 |
| chr18 | 58335402 | 58335422 | +/− | 21 | CAGCAGTGGGCCCTGATTCAG | 2048 |
| chr18 | 58335403 | 58335423 | +/− | 21 | AGCAGTGGGCCCTGATTCAGA | 2049 |
| chr18 | 58335404 | 58335424 | +/− | 21 | GCAGTGGGCCCTGATTCAGAC | 2050 |
| chr18 | 58335405 | 58335425 | +/− | 21 | CAGTGGGCCCTGATTCAGACA | 2051 |
| chr18 | 58335406 | 58335426 | +/− | 21 | AGTGGGCCCTGATTCAGACAG | 2052 |
| chr18 | 58335407 | 58335427 | +/− | 21 | GTGGGCCCTGATTCAGACAGC | 2053 |
| chr18 | 58335408 | 58335428 | +/− | 21 | TGGGCCCTGATTCAGACAGCA | 2054 |
| chr18 | 58335409 | 58335429 | +/− | 21 | GGGCCCTGATTCAGACAGCAG | 2055 |
| chr18 | 58335410 | 58335430 | +/− | 21 | GGCCCTGATTCAGACAGCAGG | 2056 |
| chr18 | 58335317 | 58335337 | +/− | 21 | TGGTCATCAGCGACTGCTGGC | 2057 |
| chr18 | 58335411 | 58335431 | +/− | 21 | GCCCTGATTCAGACAGCAGGG | 2058 |
| chr18 | 58335412 | 58335432 | +/− | 21 | CCCTGATTCAGACAGCAGGGG | 2059 |
| chr18 | 58335413 | 58335433 | +/− | 21 | CCTGATTCAGACAGCAGGGGG | 2060 |
| chr18 | 58335414 | 58335434 | +/− | 21 | CTGATTCAGACAGCAGGGGGT | 2061 |
| chr18 | 58335415 | 58335435 | +/− | 21 | TGATTCAGACAGCAGGGGGTC | 2062 |
| chr18 | 58335416 | 58335436 | +/− | 21 | GATTCAGACAGCAGGGGGTCA | 2063 |
| chr18 | 58335417 | 58335437 | +/− | 21 | ATTCAGACAGCAGGGGGTCAT | 2064 |
| chr18 | 58335418 | 58335438 | +/− | 21 | TTCAGACAGCAGGGGGTCATC | 2065 |
| chr18 | 58335419 | 58335439 | +/− | 21 | TCAGACAGCAGGGGGTCATCC | 2066 |
| chr18 | 58335420 | 58335440 | +/− | 21 | CAGACAGCAGGGGGTCATCCC | 2067 |
| chr18 | 58335421 | 58335441 | +/− | 21 | AGACAGCAGGGGGTCATCCCC | 2068 |
| chr18 | 58335422 | 58335442 | +/− | 21 | GACAGCAGGGGGTCATCCCCT | 2069 |
| chr18 | 58335423 | 58335443 | +/− | 21 | ACAGCAGGGGGTCATCCCCTA | 2070 |
| chr18 | 58335424 | 58335444 | +/− | 21 | CAGCAGGGGGTCATCCCCTAA | 2071 |
| chr18 | 58335425 | 58335445 | +/− | 21 | AGCAGGGGGTCATCCCCTAAG | 2072 |
| chr18 | 58335426 | 58335446 | +/− | 21 | GCAGGGGGTCATCCCCTAAGT | 2073 |
| chr18 | 58335427 | 58335447 | +/− | 21 | CAGGGGGTCATCCCCTAAGTG | 2074 |

TABLE 15
22-mer target-specific ASOs

| | | | | | | SEQ |
|---|---|---|---|---|---|---|
| | | | | | | ID |
| CHR | START | END | STRAND | Kmer | SEQUENCE | NO: |
| chr18 | 58335318 | 58335339 | +/− | 22 | GGTCATCAGCGACTGCTGGCTT | 2075 |
| chr18 | 58335319 | 58335340 | +/− | 22 | GTCATCAGCGACTGCTGGCTTT | 2076 |
| chr18 | 58335320 | 58335341 | +/− | 22 | TCATCAGCGACTGCTGGCTTTG | 2077 |
| chr18 | 58335321 | 58335342 | +/− | 22 | CATCAGCGACTGCTGGCTTTGT | 2078 |
| chr18 | 58335322 | 58335343 | +/− | 22 | ATCAGCGACTGCTGGCTTTGTC | 2079 |
| chr18 | 58335323 | 58335344 | +/− | 22 | TCAGCGACTGCTGGCTTTGTCT | 2080 |
| chr18 | 58335324 | 58335345 | +/− | 22 | CAGCGACTGCTGGCTTTGTCTG | 2081 |
| chr18 | 58335325 | 58335346 | +/− | 22 | AGCGACTGCTGGCTTTGTCTGG | 2082 |
| chr18 | 58335326 | 58335347 | +/− | 22 | GCGACTGCTGGCTTTGTCTGGA | 2083 |
| chr18 | 58335327 | 58335348 | +/− | 22 | CGACTGCTGGCTTTGTCTGGAT | 2084 |
| chr18 | 58335328 | 58335349 | +/− | 22 | GACTGCTGGCTTTGTCTGGATA | 2085 |
| chr18 | 58335329 | 58335350 | +/− | 22 | ACTGCTGGCTTTGTCTGGATAG | 2086 |
| chr18 | 58335330 | 58335351 | +/− | 22 | CTGCTGGCTTTGTCTGGATAGG | 2087 |
| chr18 | 58335331 | 58335352 | +/− | 22 | TGCTGGCTTTGTCTGGATAGGG | 2088 |
| chr18 | 58335332 | 58335353 | +/− | 22 | GCTGGCTTTGTCTGGATAGGGT | 2089 |
| chr18 | 58335333 | 58335354 | +/− | 22 | CTGGCTTTGTCTGGATAGGGTG | 2090 |
| chr18 | 58335334 | 58335355 | +/− | 22 | TGGCTTTGTCTGGATAGGGTGG | 2091 |
| chr18 | 58335335 | 58335356 | +/− | 22 | GGCTTTGTCTGGATAGGGTGGG | 2092 |
| chr18 | 58335336 | 58335357 | +/− | 22 | GCTTTGTCTGGATAGGGTGGGT | 2093 |
| chr18 | 58335337 | 58335358 | +/− | 22 | CTTTGTCTGGATAGGGTGGGTT | 2094 |
| chr18 | 58335338 | 58335359 | +/− | 22 | TTTGTCTGGATAGGGTGGGTTT | 2095 |
| chr18 | 58335339 | 58335360 | +/− | 22 | TTGTCTGGATAGGGTGGGTTTC | 2096 |
| chr18 | 58335340 | 58335361 | +/− | 22 | TGTCTGGATAGGGTGGGTTTCA | 2097 |
| chr18 | 58335341 | 58335362 | +/− | 22 | GTCTGGATAGGGTGGGTTTCAG | 2098 |
| chr18 | 58335342 | 58335363 | +/− | 22 | TCTGGATAGGGTGGGTTTCAGG | 2099 |
| chr18 | 58335343 | 58335364 | +/− | 22 | CTGGATAGGGTGGGTTTCAGGG | 2100 |
| chr18 | 58335344 | 58335365 | +/− | 22 | TGGATAGGGTGGGTTTCAGGGA | 2101 |
| chr18 | 58335345 | 58335366 | +/− | 22 | GGATAGGGTGGGTTTCAGGGAT | 2102 |
| chr18 | 58335346 | 58335367 | +/− | 22 | GATAGGGTGGGTTTCAGGGATT | 2103 |
| chr18 | 58335347 | 58335368 | +/− | 22 | ATAGGGTGGGTTTCAGGGATTC | 2104 |
| chr18 | 58335348 | 58335369 | +/− | 22 | TAGGGTGGGTTTCAGGGATTCT | 2105 |
| chr18 | 58335349 | 58335370 | +/− | 22 | AGGGTGGGTTTCAGGGATTCTG | 2106 |
| chr18 | 58335350 | 58335371 | +/− | 22 | GGGTGGGTTTCAGGGATTCTGA | 2107 |
| chr18 | 58335351 | 58335372 | +/− | 22 | GGTGGGTTTCAGGGATTCTGAT | 2108 |
| chr18 | 58335352 | 58335373 | +/− | 22 | GTGGGTTTCAGGGATTCTGATC | 2109 |
| chr18 | 58335353 | 58335374 | +/− | 22 | TGGGTTTCAGGGATTCTGATCT | 2110 |
| chr18 | 58335354 | 58335375 | +/− | 22 | GGGTTTCAGGGATTCTGATCTC | 2111 |
| chr18 | 58335355 | 58335376 | +/− | 22 | GGTTTCAGGGATTCTGATCTCA | 2112 |
| chr18 | 58335356 | 58335377 | +/− | 22 | GTTTCAGGGATTCTGATCTCAC | 2113 |
| chr18 | 58335357 | 58335378 | +/− | 22 | TTTCAGGGATTCTGATCTCACG | 2114 |
| chr18 | 58335358 | 58335379 | +/− | 22 | TTCAGGGATTCTGATCTCACGT | 2115 |
| chr18 | 58335359 | 58335380 | +/− | 22 | TCAGGGATTCTGATCTCACGTC | 2116 |
| chr18 | 58335360 | 58335381 | +/− | 22 | CAGGGATTCTGATCTCACGTCA | 2117 |
| chr18 | 58335361 | 58335382 | +/− | 22 | AGGGATTCTGATCTCACGTCAC | 2118 |
| chr18 | 58335362 | 58335383 | +/− | 22 | GGGATTCTGATCTCACGTCACC | 2119 |
| chr18 | 58335363 | 58335384 | +/− | 22 | GGATTCTGATCTCACGTCACCT | 2120 |
| chr18 | 58335364 | 58335385 | +/− | 22 | GATTCTGATCTCACGTCACCTG | 2121 |
| chr18 | 58335365 | 58335386 | +/− | 22 | ATTCTGATCTCACGTCACCTGC | 2122 |
| chr18 | 58335366 | 58335387 | +/− | 22 | TTCTGATCTCACGTCACCTGCC | 2123 |
| chr18 | 58335367 | 58335388 | +/− | 22 | TCTGATCTCACGTCACCTGCCT | 2124 |
| chr18 | 58335368 | 58335389 | +/− | 22 | CTGATCTCACGTCACCTGCCTT | 2125 |
| chr18 | 58335369 | 58335390 | +/− | 22 | TGATCTCACGTCACCTGCCTTA | 2126 |
| chr18 | 58335370 | 58335391 | +/− | 22 | GATCTCACGTCACCTGCCTTAC | 2127 |
| chr18 | 58335371 | 58335392 | +/− | 22 | ATCTCACGTCACCTGCCTTACA | 2128 |
| chr18 | 58335372 | 58335393 | +/− | 22 | TCTCACGTCACCTGCCTTACAG | 2129 |
| chr18 | 58335373 | 58335394 | +/− | 22 | CTCACGTCACCTGCCTTACAGC | 2130 |
| chr18 | 58335374 | 58335395 | +/− | 22 | TCACGTCACCTGCCTTACAGCG | 2131 |
| chr18 | 58335375 | 58335396 | +/− | 22 | CACGTCACCTGCCTTACAGCGC | 2132 |
| chr18 | 58335376 | 58335397 | +/− | 22 | ACGTCACCTGCCTTACAGCGCT | 2133 |
| chr18 | 58335377 | 58335398 | +/− | 22 | CGTCACCTGCCTTACAGCGCTG | 2134 |
| chr18 | 58335378 | 58335399 | +/− | 22 | GTCACCTGCCTTACAGCGCTGC | 2135 |
| chr18 | 58335379 | 58335400 | +/− | 22 | TCACCTGCCTTACAGCGCTGCC | 2136 |
| chr18 | 58335380 | 58335401 | +/− | 22 | CACCTGCCTTACAGCGCTGCCA | 2137 |
| chr18 | 58335381 | 58335402 | +/− | 22 | ACCTGCCTTACAGCGCTGCCAC | 2138 |
| chr18 | 58335382 | 58335403 | +/− | 22 | CCTGCCTTACAGCGCTGCCACA | 2139 |
| chr18 | 58335383 | 58335404 | +/− | 22 | CTGCCTTACAGCGCTGCCACAG | 2140 |
| chr18 | 58335384 | 58335405 | +/− | 22 | TGCCTTACAGCGCTGCCACAGC | 2141 |
| chr18 | 58335385 | 58335406 | +/− | 22 | GCCTTACAGCGCTGCCACAGCA | 2142 |
| chr18 | 58335386 | 58335407 | +/− | 22 | CCTTACAGCGCTGCCACAGCAG | 2143 |
| chr18 | 58335387 | 58335408 | +/− | 22 | CTTACAGCGCTGCCACAGCAGT | 2144 |
| chr18 | 58335388 | 58335409 | +/− | 22 | TTACAGCGCTGCCACAGCAGTG | 2145 |
| chr18 | 58335389 | 58335410 | +/− | 22 | TACAGCGCTGCCACAGCAGTGG | 2146 |
| chr18 | 58335390 | 58335411 | +/− | 22 | ACAGCGCTGCCACAGCAGTGGG | 2147 |
| chr18 | 58335391 | 58335412 | +/− | 22 | CAGCGCTGCCACAGCAGTGGGC | 2148 |
| chr18 | 58335392 | 58335413 | +/− | 22 | AGCGCTGCCACAGCAGTGGGCC | 2149 |
| chr18 | 58335393 | 58335414 | +/− | 22 | GCGCTGCCACAGCAGTGGGCCC | 2150 |
| chr18 | 58335394 | 58335415 | +/− | 22 | CGCTGCCACAGCAGTGGGCCCT | 2151 |
| chr18 | 58335395 | 58335416 | +/− | 22 | GCTGCCACAGCAGTGGGCCCTG | 2152 |
| chr18 | 58335396 | 58335417 | +/− | 22 | CTGCCACAGCAGTGGGCCCTGA | 2153 |
| chr18 | 58335397 | 58335418 | +/− | 22 | TGCCACAGCAGTGGGCCCTGAT | 2154 |
| chr18 | 58335398 | 58335419 | +/− | 22 | GCCACAGCAGTGGGCCCTGATT | 2155 |
| chr18 | 58335399 | 58335420 | +/− | 22 | CCACAGCAGTGGGCCCTGATTC | 2156 |
| chr18 | 58335400 | 58335421 | +/− | 22 | CACAGCAGTGGGCCCTGATTCA | 2157 |
| chr18 | 58335401 | 58335422 | +/− | 22 | ACAGCAGTGGGCCCTGATTCAG | 2158 |
| chr18 | 58335402 | 58335423 | +/− | 22 | CAGCAGTGGGCCCTGATTCAGA | 2159 |
| chr18 | 58335403 | 58335424 | +/− | 22 | AGCAGTGGGCCCTGATTCAGAC | 2160 |
| chr18 | 58335404 | 58335425 | +/− | 22 | GCAGTGGGCCCTGATTCAGACA | 2161 |
| chr18 | 58335405 | 58335426 | +/− | 22 | CAGTGGGCCCTGATTCAGACAG | 2162 |
| chr18 | 58335406 | 58335427 | +/− | 22 | AGTGGGCCCTGATTCAGACAGC | 2163 |
| chr18 | 58335407 | 58335428 | +/− | 22 | GTGGGCCCTGATTCAGACAGCA | 2164 |
| chr18 | 58335408 | 58335429 | +/− | 22 | TGGGCCCTGATTCAGACAGCAG | 2165 |
| chr18 | 58335409 | 58335430 | +/− | 22 | GGGCCCTGATTCAGACAGCAGG | 2166 |
| chr18 | 58335317 | 58335338 | +/− | 22 | TGGTCATCAGCGACTGCTGGCT | 2167 |
| chr18 | 58335410 | 58335431 | +/− | 22 | GGCCCTGATTCAGACAGCAGGG | 2168 |
| chr18 | 58335411 | 58335432 | +/− | 22 | GCCCTGATTCAGACAGCAGGGG | 2169 |
| chr18 | 58335412 | 58335433 | +/− | 22 | CCCTGATTCAGACAGCAGGGGG | 2170 |
| chr18 | 58335413 | 58335434 | +/− | 22 | CCTGATTCAGACAGCAGGGGGT | 2171 |
| chr18 | 58335414 | 58335435 | +/− | 22 | CTGATTCAGACAGCAGGGGGTC | 2172 |
| chr18 | 58335415 | 58335436 | +/− | 22 | TGATTCAGACAGCAGGGGGTCA | 2173 |
| chr18 | 58335416 | 58335437 | +/− | 22 | GATTCAGACAGCAGGGGGTCAT | 2174 |
| chr18 | 58335417 | 58335438 | +/− | 22 | ATTCAGACAGCAGGGGGTCATC | 2175 |
| chr18 | 58335418 | 58335439 | +/− | 22 | TTCAGACAGCAGGGGGTCATCC | 2176 |
| chr18 | 58335419 | 58335440 | +/− | 22 | TCAGACAGCAGGGGGTCATCCC | 2177 |
| chr18 | 58335420 | 58335441 | +/− | 22 | CAGACAGCAGGGGGTCATCCCC | 2178 |
| chr18 | 58335421 | 58335442 | +/− | 22 | AGACAGCAGGGGGTCATCCCCT | 2179 |
| chr18 | 58335422 | 58335443 | +/− | 22 | GACAGCAGGGGGTCATCCCCTA | 2180 |
| chr18 | 58335423 | 58335444 | +/− | 22 | ACAGCAGGGGGTCATCCCCTAA | 2181 |
| chr18 | 58335424 | 58335445 | +/− | 22 | CAGCAGGGGGTCATCCCCTAAG | 2182 |
| chr18 | 58335425 | 58335446 | +/− | 22 | AGCAGGGGGTCATCCCCTAAGT | 2183 |
| chr18 | 58335426 | 58335447 | +/− | 22 | GCAGGGGGTCATCCCCTAAGTG | 2184 |

Also provided herein are some additional target-specific ASOs (Tables 16-33) which may be used methods and/or systems of the present disclosure. The ASOs may be used to induce an isoform switch or modulate (e.g., inhibit or enhance) the biological activity of a specific isoform of one or several of the genes described above or elsewhere herein (e.g., genes comprising one or more of NEDD4L (ENV2), MAP3K7 (ENV3), NFYA (ENV11), ESYT2 (ENV21), MARK2 (ENV18), ST7 (ENV19), ARVCF (ENV22), SYTL2 (ENV17), R3HDM1 (ENV23), COL4A3BP (ENV9), TANGO2 (ENV6), SEPT9 (ENV15), ROBO1 (ENV4), FAM122B (ENV5), CD47 (ENV13), LSR (ENV20), PBX1 (ENV 16), EPB41 (ENV14), ADAM15 (ENV7),EPB41L1 (ENV8), ABI1 (ENV10), FLNB (ENV1), CTNND1 (ENV12), GPR160 (ENV24), ITGB3BP (ENV25), INCENP (ENV26), DENND1B (ENV27), CA12 (ENV28)).

As discussed above, oligonucleotide sequences comprised in a table may be specific for a single target, or for more than one target. In some cases, oligonucleotide sequences comprised in more than one tables are specific for a single target. For example, oligonucleotide sequences comprised in Tables 16-33 may each be specific for a single target. The targets may be the same as or differ from the target(s) which the oligonucleotide sequences comprised in Tables 2-15 are specific for. The target(s) may be one or more genes described above or elsewhere herein. In some cases, ASOs included in Table 16 are specific for NEDD4L (ENV2). In some cases, ASOs included in Table 17 are specific for MAP3K7 (ENV3). In some cases, ASOs included in Table 18 are specific for ROBO1 (ENV4). In some cases, ASOs included in Table 19 are specific for FAM122B (ENVS). In some cases, ASOs included in Table 20 are specific for TANGO2 (ENV6). In some cases, ASOs included in Table 21 are specific for ADAM15 (ENV7). In some cases, ASOs included in Table 22 are specific for EPB41L1 (ENV8). In some cases, ASOs included in Table 23 are specific for COL4A3BP (ENV9). In some cases, ASOs included in Table 23 are specific for ABI1 (ENV10). In some cases, ASOs included in Table 24 are specific for NFYA (ENV11). In some cases, AS Os included in Table 26 are specific for CTNND1 (ENV12). In some cases, ASOs included in Table 27 are specific for SEPT9 (ENV15). In some cases, ASOs included in Table 28 are specific for SYTL2 (ENV17). In some cases, ASOs included in Table 29 are specific for MARK2 (ENV18). In some cases, ASOs included in Table 30 are specific for ST7 (ENV19). In some cases, ASOs included in Table 31 are specific for ESYT2 (ENV21). In some cases, ASOs included in Table 32 are specific for ARVCF (ENV22). In some cases, ASOs included in Table 33 are specific for R3HDM1 (ENV23).

Lengthy table referenced here
US20220112497A1-20220414-T00001
Please refer to the end of the specification for access instructions.

Lengthy table referenced here
US20220112497A1-20220414-T00002
Please refer to the end of the specification for access instructions.

Lengthy table referenced here
US20220112497A1-20220414-T00003
Please refer to the end of the specification for access instructions.

Lengthy table referenced here
US20220112497A1-20220414-T00004
Please refer to the end of the specification for access instructions.

Lengthy table referenced here
US20220112497A1-20220414-T00005
Please refer to the end of the specification for access instructions.

Lengthy table referenced here
US20220112497A1-20220414-T00006
Please refer to the end of the specification for access instructions.

Lengthy table referenced here
US20220112497A1-20220414-T00007
Please refer to the end of the specification for access instructions.

Lengthy table referenced here
US20220112497A1-20220414-T00008
Please refer to the end of the specification for access instructions.

Lengthy table referenced here
US20220112497A1-20220414-T00009
Please refer to the end of the specification for access instructions.

Lengthy table referenced here
US20220112497A1-20220414-T00010
Please refer to the end of the specification for access instructions.

Lengthy table referenced here
US20220112497A1-20220414-T00011
Please refer to the end of the specification for access instructions.

Lengthy table referenced here
US20220112497A1-20220414-T00012
Please refer to the end of the specification for access instructions.

Lengthy table referenced here
US20220112497A1-20220414-T00013
Please refer to the end of the specification for access instructions.

Lengthy table referenced here
US20220112497A1-20220414-T00014
Please refer to the end of the specification for access instructions.

Lengthy table referenced here
US20220112497A1-20220414-T00015
Please refer to the end of the specification for access instructions.

Lengthy table referenced here
US20220112497A1-20220414-T00016
Please refer to the end of the specification for access instructions.

Lengthy table referenced here
US20220112497A1-20220414-T00017
Please refer to the end of the specification for access instructions.

Lengthy table referenced here
US20220112497A1-20220414-T00018
Please refer to the end of the specification for access instructions.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

LENGTHY TABLES
The patent application contains a lengthy table section. A copy of the table is available in electronic form from the USPTO web site (https://seqdata.uspto.gov/?pageRequest=docDetail&DocID=US20220112497A1). An electronic copy of the table will also be available from the USPTO upon request and payment of the fee set forth in 37 CFR 1.19(b)(3).

1.-155. (canceled)

156. An antisense oligonucleotide comprising a sequence selected from the group consisting of:
(SEQ ID NO: 1)
5′-GTGGGTTTCAGGGATTCTGA-3′,
(SEQ ID NO: 2)
5′-CCCTGATTCAGACAGCAGGG-3′,
(SEQ ID NO: 3)
5′-GCTGGCTTTGTCTGGATAGG-3′,
(SEQ ID NO: 4)
5′-TCTCACGTCACCTGCCTTAC-3′,
and
(SEQ ID NO: 5)
5′-AGCGCTGCCACAGCAGTGGG-3′,
or a complement thereof.

157. The antisense oligonucleotide of claim 156, wherein the oligonucleotide is capable of modulating splicing of NEDD4L mRNA in a cell.

158. The antisense oligonucleotide of claim 157, wherein the modulation of splicing comprises promoting a splicing switch.

159. The antisense oligonucleotide of claim 156, wherein said oligonucleotide comprises 20-50 nucleotides.

160. The antisense oligonucleotide of claim 159, wherein said oligonucleotide comprises 25-30 nucleotides.

161. The antisense oligonucleotide of claim 156, wherein said oligonucleotide comprises deoxyribonucleic acid (DNA), ribonucleic acid (RNA), PNA, or a combination or hybrid thereof.

162. The antisense compound of claim 156, wherein the oligonucleotide comprises one or more modified oligonucleotides.

163. The antisense oligonucleotide of claim 156, wherein the antisense oligonucleotide comprises at least one modified internucleoside linkage.

164. The antisense oligonucleotide of claim 163, wherein all internucleoside linkages are modified.

165. The antisense oligonucleotide of claim 163, wherein the modified internucleoside linkage comprises a phosphorothioate linkage.

166. The antisense oligonucleotide of claim 156, wherein the antisense oligonucleotide comprises one or more modified nucleoside selected from the group consisting of 2′-O-methoxyethyl nucleoside, 2′-fluoro nucleoside, 2′-dimethylaminooxyethoxy nucleoside, T-dimethylaminoethoxyethoxy nucleoside, 2′-guanidinium nucleoside, 2′-O-guanidinium ethyl nucleoside, T-carbamate nucleoside, 2′aminooxy nucleoside, T-acetamido nucleoside, and locked nucleic acid.

167. A pharmaceutically acceptable composition comprising an antisense oligonucleotide according to claim 156, and a pharmaceutically acceptable diluent or carrier.

168. A method of treating cancer in a patient in need thereof, comprising: administering to the patient a therapeutically effective amount of the pharmaceutical composition of claim 167, wherein the antisense oligonucleotide binds to a segment of a pre-mRNA which is encoded by NEDD4L and modulates splicing in the pre-mRNA.

169. The method of claim 168, wherein the cancer comprises lung cancer, kidney cancer, or breast cancer.

170. The method of claim 168, wherein the breast cancer is triple-negative breast cancer.

171. A method of modulating splicing of a NEDD4L pre-mRNA in a cell comprising: contacting the cell with an antisense compound selected from the group consisting of:
(SEQ ID NO: 1)
5′-GTGGGTTTCAGGGATTCTGA-3′,
(SEQ ID NO: 2)
5′-CCCTGATTCAGACAGCAGGG-3′,
(SEQ ID NO: 3)
5′-GCTGGCTTTGTCTGGATAGG-3′,
(SEQ ID NO: 4)
5′-TCTCACGTCACCTGCCTTAC-3′,
and
(SEQ ID NO: 5)
5′-AGCGCTGCCACAGCAGTGGG-3′,
or a complement thereof;
thereby modulating splicing in the NEDD4L pre-mRNA of the cell.

172. The method of claim 171, wherein the modulation of splicing comprises promoting a splicing switch in said pre-mRNA.

173. The method of claim 171, wherein the modulation of splicing comprises promoting inclusion of an exon in a pre-mRNA isoform.

174. A method of treating a patient having a cancer showing disease-specific splicing events of the pre-mRNA encoded by the NEDD4L gene resulting in a short pre-mRNA isoform, comprising:
contacting the cell of the patient with an ASO, wherein the ASO binds to a segment of the pre-mRNA and modulates splicing of the pre-mRNA from the short isoform to a long isoform, thereby treating the cancer.

175. The method of claim 174, wherein the ASO promotes inclusion of an excluded middle exon of an identified exon trio in the pre-mRNA.
